(19) United States
(12) Patent Application Publication
(10) Pub. No.: US 2013/0273157 A1
(43) Pub. Date: Oct. 17, 2013

(54) ORALLY DISINTEGRATING TABLET

(71) Applicants: Shiro Ishii, Osaka (JP); Yutaka Ebisawa, Osaka (JP); Takayuki Okabe, Osaka (JP)

(72) Inventors: Shiro Ishii, Osaka (JP); Yutaka Ebisawa, Osaka (JP); Takayuki Okabe, Osaka (JP)

(73) Assignee: TAKEDA PHARMACEUTICAL COMPANY LIMITED, Osaka-shi, Osaka (JP)

(21) Appl. No.: 13/997,862

(22) PCT Filed: Dec. 26, 2011
(86) PCT No.: PCT/JP2011/080568

§ 371 Date: Jun. 25, 2013

(51) Int. Cl. A61K 9/20; A61K 31/4439

(52) U.S. Cl. CPC A61K 9/2081; A61K 31/4439
USPC 424/465; 514/338

(57) ABSTRACT

A orally disintegrating tablet is obtained by tableting fine granules showing controlled release of lansoprazole and an additive, which is capable of suppressing breakage of the fine granules during tableting, and can control the release of lansoprazole for a long time, and can maintain a therapeutically effective concentration for a prolonged time, and shows superior disintegration property in the oral cavity.

### TECHNICAL FIELD OF THE INVENTION

The present invention relates to an orally disintegrating tablet showing controlled release of an active ingredient.

### BACKGROUND OF THE INVENTION

With an aging population and their changes in life environment, it is desired to develop an orally disintegrating tablet capable of being administered without water, retaining the convenience for use which is a characteristic of a tablet, and being administered on demand easily, anytime and anywhere, without water.

When the pharmaceutically active ingredient or an additive has a bitter taste, masking of the bitter taste by coating is preferable for drug compliance. When the pharmaceutically active ingredient is easily decomposed by an acid, it is necessary to coat the ingredient to prevent decomposition by the gastric acid and ensure sufficient delivery to the intestine. To solve these problems, coated tablets, capsules and the like are generally used.

To meet these requirements, tablets containing coated fine granules have conventionally been developed. For example, a rapidly disintegratable multiparticular tablet comprising a pharmaceutically active ingredient in the form of coated fine particles (patent document 1) and orally disintegrating tablets containing coated fine granules (patent documents 2 and 3) have been disclosed.

During the production of solid preparations such as tablet containing coated fine granules and the like, fine granules may be broken during tableting as evidenced by partial destruction of a coating layer of fine granules and the like, resulting in problems such as a decreased masking effect on the aforementioned bitter taste, acid resistance and the like.

In recent years, the development of an oral formulation showing maintained efficacy by 1 or 2 times of administration per day is desired to improve QOL, and attempts have been made to design a sustained-release preparation by formulation ideas and modifying the kinetics. As the dosage form of an oral sustained-release preparation, various release control systems such as a controlled release formulation based on the control of diffusion of the compound by a controlled release coating film or matrix, a controlled release formulation based on a matrix (base) corrosion, a controlled release formulation of a pH-dependent compound, a time-controlled release formulation that releases the compound after a certain lag time and the like have been developed and applied (patent document 4).

In a preparation containing a drug unstable to acid, such as a benzimidazole compound having a proton pump inhibitory action, as an active ingredient, an enteric coating needs to be applied. On the other hand, rapid disintegration is necessary in the small intestine. Therefore, formulation of granules or fine granules is preferable, since they have a larger surface area than tablets, and are disintegrated or dissolved rapidly. In the case of tablets, moreover, compact tablets are desirable (patent document 5).

Tablets, granules and fine granules after oral administration pass through the gastrointestinal tract from the stomach, duodenum, jejunum, ileum to the large intestine while releasing the active ingredient to allow absorption of thereof from the respective absorption sites.

### DOCUMENT LIST

### Patent Documents

patent document 1: JP-A-6-502194
patent document 2: JP-A-2000-281564
patent document 3: JP-A-2000-103731
patent document 4: JP-A-2004-292427
patent document 5: JP-A-62-277322

### SUMMARY OF THE INVENTION

### Problems to be Solved by the Invention

The present invention aims to provide an orally disintegrating tablet containing fine granules showing controlled release of a pharmaceutically active ingredient, which is capable of suppressing breakage of the fine granules during tableting in the production of the orally disintegrating solid preparation and controlling dissolution property of a pharmaceutically active ingredient.

### Means of Solving the Problems

Accordingly, the present invention provides the following:

[1] an orally disintegrating tablet comprising
(i) fine granules showing controlled release of a pharmaceutically active ingredient, which comprises fine granules containing a pharmaceutically active ingredient and a coating layer comprising a methacrylic acid/methyl acrylate/methyl methacrylate copolymer, wherein the fine granules containing a pharmaceutically active ingredient are coated with more than 80 wt % and not more than 300 wt % of the copolymer (sometimes to be referred to as “fine granules (i)” in the present specification), and
(ii) fine granules showing controlled release of a pharmaceutically active ingredient, which comprises the pharmaceutically active ingredient and a coating layer comprising (a) an ethyl acrylate/methyl methacrylate copolymer, and (b) one or more kinds of polymers selected from the group consisting of methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate (sometimes to be referred to as “fine granules (ii)” in the present specification), wherein the fine granules (i) and fine granules (ii) have an average particle size of not more than 500 μm, and the pharmaceutically active ingredient is lansoprazole or an optically active form thereof or a salt thereof (sometimes to be referred to as “tablet (I)” in the present specification),
[2] an orally disintegrating tablet comprising
(i) fine granules showing controlled release of a pharmaceutically active ingredient, which comprises a pharmaceutically active ingredient and a coating layer comprising (a) a methacrylic acid/methyl acrylate/methyl methacrylate copolymer, and (b) one or more kinds of polymers selected from the group consisting of an ethyl acrylate/methyl methacrylate copolymer, polyvinyl acetate and ethylcellulose (sometimes to be referred to as “fine granules (i)” in the present specification), and
(ii) fine granules showing controlled release of a pharmaceutically active ingredient, which comprises a pharmaceutically active ingredient and a coating layer comprising (a) an ethyl acrylate/methyl methacrylate copolymer, and (b) one or more kinds of polymers selected from the group consisting of methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate (sometimes to be referred to as “fine granules (ii)” in the present specification), wherein the fine granules (i) and fine granules (ii) have an average particle size of not more than 500 μm, and the pharmaceutically active ingredient is lansoprazole or an optically active form thereof or a salt thereof (sometimes to be referred to as “tablet (II)” in the present specification),
[3] the orally disintegrating tablet of the aforementioned [1] or [2], wherein the coating layers of fine granules (i) and
(ii) comprise a plasticizer,
[4] the orally disintegrating tablet of the aforementioned [1], wherein the coating layer of fine granules (i) has a coating thickness of 35-70 μm,
[5] the orally disintegrating tablet of the aforementioned [1] or [2], wherein the pharmaceutically active ingredient is an optically active R form of lansoprazole,
[6] the orally disintegrating tablet of the aforementioned [1] or [2], further comprising an additive,
[7] the orally disintegrating tablet of the aforementioned [6], wherein the additive is a water-soluble sugar alcohol,
[8] the orally disintegrating tablet of the aforementioned [1] or [2], wherein the coating layers of fine granules (i) and
(ii) are formed on an intermediate layer,
[9] the orally disintegrating tablet of the aforementioned [1] or [2], wherein the coating layer comprising polyethylene glycol, (a) an ethyl acrylate/methyl methacrylate copolymer and (b) one or more kinds of polymers selected from the group consisting of methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate is further formed on each coating layer of fine granules (i) and fine granules (ii).

### Effect of the Invention

Since the orally disintegrating tablet of the present invention shows suppressed breakage of fine granules (i.e., “fine granules (i)” and “fine granules (ii)” contained in tablet (I), and “fine granules (i)” and “fine granules (ii)” contained in tablet (II)), the acid resistance of a medicament unstable to acid, such as lansoprazole, can be retained and the release of the pharmaceutically active ingredient can be controlled as desired.

Since the orally disintegrating tablet of the present invention containing two kinds of fine granules showing different releaseability of the pharmaceutically active ingredient can control the release of a pharmaceutically active ingredient for a long time, a therapeutically effective concentration can be maintained for a prolonged time. Therefore administration frequency can be reduced, and effectiveness of treatment at a low dose and reduction of side effects caused by the rise of blood concentration can be ensured.

Since the orally disintegrating tablet of the present invention has hardness to be possessed as a tablet, and shows superior disintegration property or dissolution property in the oral cavity, it is used for the treatment of diseases as a formulation conveniently taken by elderly persons and children even without water. In addition, since the fine granules comprising the pharmaceutically active ingredient having a size preventing rough or dusty texture are blended, the tablet is smooth in the mouth.

### BRIEF DESCRIPTION OF THE DRAWINGS

FIG. 1 shows the results of Experimental Example 1.

FIG. 2 shows the results of Experimental Example 2.

FIG. 3 shows the results of Experimental Example 3.

FIG. 4 shows the results of Experimental Example 4.

FIG. 5 shows the results of Experimental Example 5.

FIG. 6 shows the results of Experimental Example 6.

FIG. 7 shows the results of Experimental Example 7.

FIG. 8 shows the results of Experimental Example 8.

FIG. 9 shows the results of Experimental Example 9.

FIG. 10 shows the results of Experimental Example 10.

FIG. 11 shows the results of Experimental Example 11.

FIG. 12 shows the results of Experimental Example 12.

FIG. 13 shows the results of Experimental Example 13.

FIG. 14 shows the results of Experimental Example 14.

FIG. 15 shows the results of Experimental Example 15.

FIG. 16 shows the results of Experimental Example 16.

FIG. 17 shows the results of Experimental Example 17.

FIG. 18 shows the results of Experimental Example 18.

FIG. 19 shows the results of Experimental Example 19.

FIG. 20 shows the results of Experimental Example 20.

FIG. 21 shows the results of Experimental Example 21.

FIG. 22 shows the results of Experimental Example 22.

### DETAILED DESCRIPTION OF THE INVENTION

The present invention is explained in detail in the following.

The pharmaceutically active ingredient to be used in the present invention is lansoprazole, i.e., 2-[[[3-methyl-4-(2,2,2-trifluoroethoxy)-2-pyridinyl]methyl]sulfinyl]-1H-benzimidazole. It may be a racemate or an optically active form such as R-form, S-form and the like. Particularly, (R)-2-[[[3-methyl-4-(2,2,2-trifluoroethoxy)-2-pyridinyl]methyl]sulfinyl]-1H-benzimidazole is preferable.

In the present invention, the pharmaceutically active ingredient may be a salt of lansoprazole or an optically active form thereof. The salt is preferably a pharmaceutically acceptable salt. Examples thereof include salts with inorganic base, salts with organic base, salts with basic amino acid, and the like.

Preferable examples of the salt with inorganic base include alkali metal salts such as sodium salt, potassium salt and the like; alkaline earth metal salts such as calcium salt, magnesium salt and the like; ammonium salt and the like.

Preferable examples of the salt with organic base include salts with alkylamines (trimethylamine, triethylamine etc.), heterocyclic amines (pyridine, picoline etc.), alkanolamines (ethanolamine, diethanolamine, triethanolamine etc.), dicyclohexylamine, N,N′-dibenzylethylenediamine and the like.

Preferable examples of the salt with basic amino acid include salts with arginine, lysine, ornithine and the like.

Of these salts, alkali metal salts and alkaline earth metal salts are preferable. Sodium salt is particularly preferable.

Lansoprazole can be produced according to a method known per se, for example, the method described in JP-A-61-50978, U.S. Pat. No. 4,628,098, JP-A-10-195068, WO 98/21201 or the like or a method analogous thereto. In addition, the optically active form can be produced according to a method such as optical resolution (fractional recrystallization, chiral column method, diastereomer method, a method using microorganism or enzyme, and the like), asymmetric oxidation and the like. For example, R form lansoprazole can also be produced according to the method described in WO 00/78745, WO 01/83473, WO 01/87874 and WO 02/44167.

The pharmaceutically active ingredient may be diluted with a diluent and the like generally used in the fields of medicine, food and the like.

The total amount of the aforementioned pharmaceutically active ingredient is, for example, about 0.01- about 50 parts by weight, preferably about 0.05- about 30 parts by weight, per 100 parts by weight of the formulation of the present invention.

In the present invention, the “fine granules containing a pharmaceutically active ingredient” means particles having a coating layer containing a pharmaceutically active ingredient on the “core” and optionally having an “intermediate layer” on the particles, or particles obtained by using a pharmaceutically active ingredient, and excipient, polymer and the like, which are generally used for formulation, and optionally having an “intermediate layer” on the particles. The “core” and the “intermediate layer” are to be mentioned below.

In the present invention, “fine granules (i)” and “fine granules (ii)” contained in tablet (I), as well as the “fine granules (i)” and “fine granules (ii)” contained in tablet (II) (hereinafter sometimes to be collectively referred to as “fine granules”) show controlled release of a pharmaceutically active ingredient. They are particles comprised of fine granules containing a pharmaceutically active ingredient and a controlled release coating film coated thereon.

In the present invention, the “fine granules” is as defined in the Japanese Pharmacopoeia, the 15th edition (a powder wherein 10% or less of the total amount of the powder passes a 75 μm sieve). The average particle size of the fine granules in the formulation of the present invention desirably has an average particle size of about 500 μm or below, preferably about 400 μm or below, in order to prevent rough or powdery texture during administration of the tablet of the present invention. For example, it is about 100- about 500 μm, preferably about 100- about 400 μm.

Unless otherwise specified, the “average particle size” means a volume median diameter (median diameter: a particle diameter corresponding to 50% of cumulative distribution). Examples include a laser diffraction particle size distribution measuring method, specifically, a method using a laser diffraction particle size distribution analyzer HEROS RODOS (manufactured by Sympatec, Germany).

The “fine granules (i)” contained in tablet (I) of the present invention are those showing the controlled release of a pharmaceutically active ingredient, which comprise fine granules containing a pharmaceutically active ingredient and a coating layer comprising a methacrylic acid/methyl acrylate/methyl methacrylate copolymer, wherein the fine granules containing a pharmaceutically active ingredient are coated with more than 80 wt % and not more than 300 wt % of the copolymer.

In other words, “fine granules (i)” of tablet (I) are particles showing controlled release of a pharmaceutically active ingredient, and having a controlled release coating film having a methacrylic acid/methyl acrylate/methyl methacrylate copolymer content of more than 80 wt % and not more than 300 wt %, preferably more than 80 wt % and not more than 250 wt %, more preferably more than 85 wt % and not more than 200 wt %, still more preferably more than 90 wt % and not more than 150 wt %; in another embodiment, it is more than 80 wt % and not more than 300 wt %, preferably more than 80 wt % and not more than 250 wt %, more preferably not less than 85 wt % and not more than 200 wt %, still more preferably not less than 85 wt % and not more than 170 wt %, relative to the fine granules containing a pharmaceutically active ingredient.

Examples of such controlled release coating film include methacrylic acid/methyl acrylate/methyl methacrylate copolymer (Eudragit FS30D manufactured by Evonik).

When the fine granules containing a pharmaceutically active ingredient have a core, the content of the methacrylic acid/methyl acrylate/methyl methacrylate copolymer relative to the “core” is more than 350 wt % and not more than 1350 wt %, preferably more than 350 wt % and not more than 1150 wt %, more preferably more than 375 wt % and not more than 900 wt %, still more preferably more than 400 wt % and not more than 700 wt %; in another embodiment, it is more than 350 wt % and not more than 1350 wt %, preferably more than 350 wt % and not more than 1150 wt %, more preferably not less than 375 wt % and not more than 900 wt %, still more preferably not less than 400 wt % and not more than 800 wt %.

Moreover, the coating layer of the methacrylic acid/methyl acrylate/methyl methacrylate copolymer of the “fine granules (i)” of tablet (I) has a coating thickness of preferably about 35- about 70 μm, more preferably about 35.5- about 60 μm, still more preferably 36-55 μm; in another embodiment, it is preferably about 35- about 70 μm, more preferably about 35.5- about 67.5 μm, still more preferably about 36- about 65 μm.

The “coating thickness” means the theoretical calculation values obtained as follows.

1: The average particle size of the core is measured by a laser diffraction particle size analyzer HERDS RODOS (manufactured by Sympatec (Germany)), from which the volume of the core is calculated.
2: Assuming the volume increase rate is the same as the weight increase rate, the volume of the granules obtained by coating the core with a coating layer is calculated.
3: The particle size of the granules is calculated from the volume thereof.
4: The thickness of the coating layer is calculated from the particle size of the core and the particle size of the granules.

For example, when Nonpareil (manufactured by Freund Corporation, spherical granules of crystalline cellulose and lactose) is used as a core, which is coated with a pharmaceutically active ingredient, the film thickness of the pharmaceutically active ingredient-containing layer is calculated as follows.

The volume of Nonpareil is calculated from the average particle size of Nonpareil measured by a laser diffraction particle size analyzer HERDS RODOS (manufactured by Sympatec (Germany)). In the case of pharmaceutically active ingredient-containing fine granules containing 20 mg of Nonpareil and 40 mg of a pharmaceutically active ingredient-containing layer, assuming that the volume increase rate is same as the particle weight increase rate, the volume of Nonpareil multiplied by 60/20 equals the volume of the fine granules containing a pharmaceutically active ingredient. The radius of the granules containing a pharmaceutically active ingredient is calculated from the calculated volume. The radius of the core particles Nonpareil is subtracted from the radius of the pharmaceutically active ingredient-containing granules to give the thickness of the pharmaceutically active ingredient-containing layer. The “film thickness” in the context of the present invention refers to a theoretically calculated value obtained by such method.

The “fine granules (ii)” contained in tablet (I) and tablet (II) of the present invention contain a pharmaceutically active ingredient, and are fine granules showing controlled release of a pharmaceutically active ingredient, which comprises a coating layer comprising (a) an ethyl acrylate/methyl methacrylate copolymer and (b) one or more kinds (preferably one or two kinds) of polymers selected from the group consisting of a methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate.

In other words, the “fine granules (ii)” of tablet (I) and tablet (II) are particles showing controlled release of a pharmaceutically active ingredient, which comprises fine granules containing a pharmaceutically active ingredient and a controlled release coating film comprising (a) an ethyl acrylate/methyl methacrylate copolymer and (b) one or more kinds (preferably one or two kinds) of polymers selected from the group consisting of methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate.

Examples of such polymer include (a) ethyl acrylate/methyl methacrylate copolymer (Eudragit NE30D, manufactured by Evonik), (b) methacrylic acid/ethyl acrylate copolymer (Eudragit L100-55 or Eudragit L30D-55, manufactured by Evonik), hypromellose phthalate (HP-55, HP-50, manufactured by Shin-Etsu Chemical Co., Ltd.), carboxymethylethylcellulose (CMEC, manufactured by Freund Corporation), polyvinyl acetate phthalate, hydroxypropyl methylcellulose acetate succinate (HPMCAS manufactured by Shin-Etsu Chemical Co., Ltd.), and cellulose acetate phthalate.

In “fine granules (ii)” of tablet (I) and tablet (II), (b) to be combined with (a) preferably includes methacrylic acid/ethyl acrylate copolymer (Eudragit L100-55 or Eudragit L30D-55, manufactured by Evonik), hypromellose phthalate (HP-55, HP-50, manufactured by Shin-Etsu Chemical Co., Ltd.), carboxymethylethylcellulose (CMEC, manufactured by Freund Corporation), hydroxypropyl methylcellulose acetate succinate (HPMCAS manufactured by Shin-Etsu Chemical Co., Ltd.), more preferably, methacrylic acid/ethyl acrylate copolymer (Eudragit L100-55 or Eudragit L30D-55, manufactured by Evonik), hypromellose phthalate (HP-55, HP-50, manufactured by Shin-Etsu Chemical Co., Ltd.), hydroxypropyl methylcellulose acetate succinate (HPMCAS manufactured by Shin-Etsu Chemical Co., Ltd.), more preferably methacrylic acid/ethyl acrylate copolymer (Eudragit L100-55 or Eudragit L30D-55, manufactured is by Evonik).

In “fine granules (ii)” of tablet (I) and tablet (II), a most preferable combination for the controlled release coating film is that of (a) an ethyl acrylate/methyl methacrylate copolymer and (b) a methacrylic acid/ethyl acrylate copolymer.

A preferable mixing ratio of (a) an ethyl acrylate/methyl methacrylate copolymer and (b) a methacrylic acid/ethyl acrylate copolymer in weight ratio is (a):(b)=0-20:100-80 (excluding (a)=0), preferably (a):(b)=0-15:100-85 (excluding (a)=0), more preferably (a):(b)=5-15:95-85.

The “fine granules (i)” contained in tablet (II) of the present invention are fine granules showing controlled release of a pharmaceutically active ingredient, which comprise a pharmaceutically active ingredient and a coating layer comprising (a) a methacrylic acid/methyl acrylate/methyl methacrylate copolymer, and (b) one or more kinds (preferably one or two kinds) of polymers selected from the group consisting of an ethyl acrylate/methyl methacrylate copolymer, polyvinyl acetate and ethylcellulose.

In other words, “fine granules (i)” of tablet (II) are particles showing controlled release of a pharmaceutically active ingredient, wherein fine granules containing a pharmaceutically active ingredient are coated with a controlled release coating film containing (a) a methacrylic acid/methyl acrylate/methyl methacrylate copolymer and (b) one or more kinds of polymers selected from the group consisting of ethyl acrylate/methyl methacrylate copolymer, polyvinyl acetate and ethylcellulose.

Examples of such polymer include (a) methacrylic acid/methyl acrylate/methyl methacrylate copolymer (Eudragit FS30D, manufactured by Evonik), (b) ethyl acrylate/methyl methacrylate copolymer (Eudragit NE30D, manufactured by Evonik), ethylcellulose (Aquacoat, manufactured by FMC), and polyvinyl acetate (Kollicoat SR30D, manufactured by BASF), and (b) to be combined with (a) preferably includes ethyl acrylate/methyl methacrylate copolymer (Eudragit NE30D, manufactured by Evonik), polyvinyl acetate (Kollicoat SR30D, manufactured by BASF), more preferably ethyl acrylate/methyl methacrylate copolymer (Eudragit NE30D, manufactured by Evonik).

In “fine granules (i)” of tablet (II), a most preferable combination for the controlled release coating film is that of (a) a methacrylic acid/methyl acrylate/methyl methacrylate copolymer and (b) an ethyl acrylate/methyl methacrylate copolymer. While the ratio of the polymers to be combined is not particularly limited, the weight of (b) polymer is not more than 70 wt % (preferably 10-70 wt %, more preferably 20-60 wt %, more preferably 30-50 wt %) (excluding (b)=0 wt %) when the total weight of (a) polymer and (b) polymer is 100 wt %.

In the present invention, the controlled release coating film layer includes not only a film-like coating layer but also a coating layer having a greater thickness, and further, not only a coating layer that completely covers fine granules containing a pharmaceutically active ingredient or layers inside, but also a coating layer that covers most of the fine granules containing a pharmaceutically active ingredient or layers inside, though partially not covering them. The coating layer that covers most of the fine granules containing a pharmaceutically active ingredient or layers inside covers at least 80% or more of the fine granules containing a pharmaceutically active ingredient or surface of the layers inside, preferably the entirety thereof.

In the present invention, the “fine granules” release a pharmaceutically active ingredient in a pH-dependent manner. The pH-dependent release of a pharmaceutically active ingredient can be controlled by covering the “fine granules containing a pharmaceutically active ingredient” in the present invention with the above-mentioned controlled release coating film. The controlled release coating film may consist of plural layers (preferably 2 to 4 layers). Moreover, release of a pharmaceutically active ingredient may be controlled by, in addition to the use of the above-mentioned controlled release coating film, a conventional method including dispersing a pharmaceutically active ingredient in a controlled release matrix during production of fine granules containing a pharmaceutically active ingredient.

The “fine granules” in the present invention are further allowed to contain a coating layer comprising polyethylene glycol and (a) an ethyl acrylate/methyl methacrylate copolymer and (b) one or more kinds of polymers selected from the group consisting of a methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate.

In the coating layer containing polyethylene glycol, (b) to be combined with (a) preferably includes methacrylic acid/ethyl acrylate copolymer (Eudragit L100-55 or Eudragit L30D-55, manufactured by Evonik), hypromellose phthalate (HP-55, HP-50, manufactured by Shin-Etsu Chemical Co., Ltd.), carboxymethylethylcellulose (CMEC, manufactured by Freund Corporation), hydroxypropyl methylcellulose acetate succinate (HPMCAS manufactured by Shin-Etsu Chemical Co., Ltd.), more preferably, methacrylic acid/ethyl acrylate copolymer (Eudragit L100-55 or Eudragit L30D-55, manufactured by Evonik), hypromellose phthalate (HP-55, HP-50, manufactured by Shin-Etsu Chemical Co., Ltd.), hydroxypropyl methylcellulose acetate succinate (HPMCAS manufactured by Shin-Etsu Chemical Co., Ltd.), more preferably methacrylic acid/ethyl acrylate copolymer (Eudragit L100-55 or Eudragit L30D-55, manufactured by Evonik). A preferable mixing ratio of (a) an ethyl acrylate/methyl methacrylate copolymer and (b) a methacrylic acid/ethyl acrylate copolymer in weight ratio is (a):(b)=0-20:100-80 (excluding (a)=0), preferably (a):(b)=0-15:100-85 (excluding (a)=0), more preferably (a):(b)=5-15:95-85.

The content of the polyethylene glycol is about 1- about 30 wt %, preferably about 3- about 25 wt %, more preferably about 5- about 20 wt %, relative to the weight of the polymer solid in the coating layer.

Such coating layer containing polyethylene glycol is preferably further formed, for example, on the coating layer (i.e., on a controlled release coating film) of “fine granules (i)” and “fine granules (ii)” of tablet (I) and tablet (II).

The thickness of the layer containing polyethylene glycol is not particularly limited as long as the effect of the present invention can be achieved. For example, the thickness is preferably not less than 0.5 μm and not more than 20 μm.

Such coating layer containing polyethylene glycol may also be formed with plural layers (preferably 2 to 4 layers). When multiple layers are formed, they may have different compositions, and may be formed plural times in any order with other coating layers. For example, fine granules containing a pharmaceutically active ingredient are coated with a controlled release coating film, followed by coating a film containing polyethylene glycol, followed by coating a controlled release coating film again, followed by further coating a film containing polyethylene glycol.

In the present invention, the “controlled release of a pharmaceutically active ingredient” means that the release of a drug is controlled such that one of the two kinds of fine granules showing different drug release profiles shows, in a dissolution test using a buffer test (50 mM phosphate buffer (pH 6.0) containing 5 mM Tween 20, 150 rpm, 900 mL) (basket method, USP Apparatus 1), dissolution of not less than 50-60% in 15 min, more preferably, not less than 70% in 15 min and 100% in 30 min in the test, and the other fine granules show, in a dissolution test using a buffer test (50 mM phosphate buffer (pH 7.2) containing 5 mM Tween 20, 150 rpm, 900 mL) (basket method, USP Apparatus 1), dissolution of not more than 15% in 30 min and not less than 60-70% in 120 min in the test.

In the present invention, for example, a preferable pH at which a coating layer containing methacrylic acid/methyl acrylate/methyl methacrylate copolymer in “fine granules (i)” of tablet (I) and tablet (II) starts to dissolve is not less than pH 6.0 and not more than 7.5, more preferably not less than pH 6.5 and not more than 7.3, and a preferable pH at which a coating layer containing ethyl acrylate/methyl methacrylate copolymer in “fine granules (ii)” is not less than pH 5.0 and not more than 6.0.

The coating layer of “fine granules (i)” and “fine granules (ii)” in tablet (I) and tablet (II) preferably contains a plasticizer.

Examples of the plasticizer include triethyl citrate, polyethylene glycol, diethyl phthalate, triacetine, glycerol, glycerol fatty acid ester, sesame oil, castor oil and the like, preferably triethyl citrate, polyethylene glycol, triacetine, more preferably triethyl citrate, polyethylene glycol, more preferably triethyl citrate.

The content of the plasticizer is about 1- about 30 wt %, preferably about 3- about 25 wt %, more preferably about 5- about 20 wt %, relative to the weight of the polymer solid in the coating layer.

The fine granules containing a pharmaceutically active ingredient in the present invention can be produced by the following method. As mentioned above, they can be obtained by (1) coating an inactive carrier as a core with a pharmaceutically active ingredient, or (2) granulation using a pharmaceutically active ingredient and excipient, polymer, etc. generally used for formulation.

(1) Production Method Including Coating an Inactive Carrier as a Core with a Pharmaceutically Active Ingredient

For coating of the core, for example, a mixture of the aforementioned pharmaceutically active ingredient and water-soluble polymer is used. The mixture may be a solution or a dispersion, which can be prepared by using water or an organic solvent such as ethanol and the like, or a mixture thereof.

Examples of the water-soluble polymer include hydroxypropylcellulose, hydroxypropylmethylcellulose, polyvinylpyrrolidone, polyvinyl alcohol, methylcellulose, hydroxyethylmethylcellulose and the like. Preferred are hydroxypropylcellulose, hydroxypropylmethylcellulose and polyvinylpyrrolidone, more preferred are hydroxypropylcellulose and hydroxypropylmethylcellulose.

While the concentration of the water-soluble polymer in the mixture varies depending on the proportion of the pharmaceutically active ingredient and the additive, it is generally about 0.1- about 50 wt %, preferably about 0.5- about 10 wt %, so as to maintain the binding force of the pharmaceutically active ingredient to the core, as well as to maintain the viscosity of the mixture to prevent decreased workability.

When the coating layer comprises a plurality of layers, the concentration of the pharmaceutically active ingredient in each layer may be changed successively or gradually by selecting the content or the viscosity grade of the water-soluble polymer or by coating successively with mixtures which are different in the proportions of the pharmaceutically active ingredient and the other additives in the mixtures. In this case, coating may be performed by using a mixture comprising the water-soluble polymer in an amount out of the range of about 0.1 to about 50% by weight, as long as coating layers in total contain about 0.1 to about 50% by weight of the water-soluble polymer. Further, the coating layer comprising a plurality (preferably 2 or 3) of layers may comprise inert coating film layers formed by a known method so that the inert coating film layer can block each layer comprising the pharmaceutically active ingredient.

After drying, fine granules containing a pharmaceutically active ingredient with a uniform particle size can be obtained by sieving. The form of the fine granules containing a pharmaceutically active ingredient generally corresponds to that of the core, and therefore, a composition close to a sphere can also be obtained. Regarding the sieve, for example, a No. 50 (300 μm) round sieve can be used. Fine granules containing a pharmaceutically active ingredient can be obtained by selecting from the granules that pass through the No. 50 round sieve.

When fine granules containing a pharmaceutically active ingredient are obtained by coating an inactive carrier as a core with a pharmaceutically active ingredient, the core is preferably as uniformly spherical as possible, so that the variations in the amount of coating can be minimized. Examples of the “coating method” include a rolling granulation method (e.g., centrifugal rolling granulation method, etc.), a fluidized bed granulation method (e.g., tumbling fluidized bed granulation, fluidized bed granulation, etc.), a stirring granulation method and the like. Specific example of the tumbling fluidized bed granulation method is, for example, a method using a tumbling fluidized bed coater (SPIR-A-FLOW (manufactured by Freund Corporation), MP-01 (manufactured by POWREX), and MP-10 TOKU-2 type (manufactured by POWREX)). Specific example of the centrifugal rolling granulation method is a method using a centrifugation rolling granulation apparatus (CF-mini, CF-360, manufactured by Freund Corporation). A two-step coating may be applied by combining the aforementioned two kinds of apparatuses. A spray method of the mixture can be appropriately selected according to the kind of the granulation apparatus and, for example, may be any of top spray method, bottom spray method, tangential spray system, and side spray method. Of these, the tangential spray system is preferable.

### (2) Production Method Using Pharmaceutically Active Ingredient and Excipient, Polymer and the Like Generally Used for Formulation

When the core of an inactive carrier is not used, core granules containing pharmaceutically active ingredient are obtained by the use of an excipient such as lactose, sucrose, mannitol, cornstarch, crystalline cellulose and the like and a pharmaceutically active ingredient, a binder such as hypromellose (HPMC), hydroxypropylcellulose, methylcellulose, polyvinyl alcohol, macrogol, pluronic F68, gum arabic, gelatin, starch and the like, and adding, where necessary, a disintegrant such as carboxymethylcellulose sodium, carboxymethylcellulose calcium, croscarboxymethylcellulose sodium (Ac-Di-Sol, manufactured by FMC International), polyvinylpyrrolidone, low-substituted hydroxypropylcellulose (L-HPC) and the like in a mixer granulator, a wet extrusion-granulator, a fluidized bed granulator and the like. The aforementioned coating method can also be utilized for coating of particles other than the core granules.

Another form free of use of a core made of an inert carrier is fine granules containing a pharmaceutically active ingredient that a pharmaceutically active ingredient is dispersed in controlled release matrices. Such fine granules containing a pharmaceutically active ingredient can be produced by uniformly dispersing a pharmaceutically active ingredient in a hydrophobic carrier such as wax (e.g., hydrogenated castor oil, hydrogenated rapeseed oil, stearic acid, stearyl alcohol and the like), or polyglycerol fatty acid ester and the like. Where necessary, excipients such as lactose, mannitol, cornstarch, crystalline cellulose and the like, generally used for formulation of preparations, may be dispersed together with the pharmaceutically active ingredient in controlled release matrices. Furthermore, a powder that becomes viscous gel upon contact with water, such as polyethylene oxide, crosslinking type acrylic acid polymer (Hibiswako (R)103, 104, 105, carbopol), HPMC, HPC, chitosan and the like may be dispersed in the controlled release matrix together with a pharmaceutically active ingredient and excipients.

For preparation, a method such as spray drying, spray chilling, melt spray congeal, melt granulation and the like can be used.

The “fine granules” in the present invention are also produced by coating fine granules containing a pharmaceutically active ingredient with the aforementioned controlled release coating film according to a production method similar to the “coating method” in the production method of the fine granules containing a pharmaceutically active ingredient, with the aim of the protection and the controlled release of the pharmaceutically active ingredient.

The “core” in the present invention means an inert carrier and examples thereof include (1) a spherical granulated product of crystalline cellulose and lactose, (2) a spherical crystalline cellulose having a size of 75 to 300 μm (CELPHERE, manufactured by Asahi Kasei Corporation), (3) a granule having a size of 50 to 250 μm produced from lactose (9 parts) and α-starch (1 part) by stirring granulation, (4) a micro particle having a size of 250 μm or smaller obtained by classification of microcrystalline cellulose spherical granules described in JP-A 61-213201, (5) a processed product of wax which is formed into a sphere by spray chilling or melt granulation, (6) a processed product such as a gelatin bead comprising an oil ingredient, (7) calcium silicate, (8) starch, (9) a porous particle such as chitin, cellulose, chitosan or the like, (10) a bulk powder of granulated sugar, crystalline lactose, crystalline cellulose, sodium chloride or the like, and a processed preparation thereof. Further, these cores may be produced by generally known grinding method or granulation method, and then sieved to prepare particles having the desired particle diameter.

Examples of the “spherical granulated product of crystalline cellulose and lactose” include (i) a spherical granule having a size of 100 to 200 μm produced from crystalline cellulose (3 parts) and lactose (7 parts) (e.g., Nonpareil 105 (70-140) (particle diameter: 100 to 200 μm), manufactured by Freund Corporation), (ii) a spherical granule having a size of 150 to 250 μm produced from crystalline cellulose (3 parts) and lactose (7 parts) (e.g., Nonpareil NP-7:3, manufactured by Freund Corporation), (iii) a spherical granule having a size of 100 to 200 μm produced from crystalline cellulose (4.5 parts) and lactose (5.5 parts) (e.g., Nonpareil 105T (70-140) (particle diameter: 100 to 200 μm), manufactured by Freund Corporation), (iv) a spherical granule having a size of 150 to 250 μm produced from crystalline cellulose (5 parts) and lactose (5 parts) (e.g., Nonpareil NP-5:5, manufactured by Freund Corporation) and the like.

In order to produce a formulation retaining a suitable strength and having excellent solubility, the “core” is preferably a spherical granule of crystalline cellulose and lactose, and more preferably a spherical granule of crystalline cellulose and lactose which contains 50% by weight or more of lactose. A spherical granule composed of preferably about 20- about 50 wt %, more preferably about 40- about 50 wt %, of crystalline cellulose and preferably about 50- about 80 wt %, more preferably about 50- about 60 wt %, of lactose is also preferable.

Examples of the “spherical crystalline cellulose” include CELPHERE (CP-203 (particle size 150-300 μm), CP-102 (particle size 106-212 μm), SCP-100 (particle size 75-212 μm), each manufactured by Asahi Kasei Chemicals Co., Ltd.) and the like.

The average particle size of the “core” is about 40- about 350 μm, preferably about 50- about 250 μm, more preferably about 100- about 250 μm, particularly preferably about 100- about 200 μm. The core having the aforementioned average particle size includes particles that completely pass through a No. 48 (300 μm) sieve, and pass through a No. 60 (250 μm) sieve except not more than about 5 w/w % of the whole, and remain in a No. 270 (53 μm) sieve except not more than about 10 w/w % of the whole. The specific volume of the “core” is not more than 5 ml/g, preferably not more than 4 ml/g, more preferably not more than 3 ml/g.

As the core used in the present invention, spherical crystalline cellulose or a spherical granule of crystalline cellulose and lactose is preferable, and 100-250 μm of spherical crystalline cellulose or a 100-200 μm spherical granule of crystalline cellulose (4.5 parts) and lactose (5.5 parts) is more preferable.

The “fine granules containing a pharmaceutically active ingredient” in the present invention also include particles having an intermediate layer, which is a coating with a polymer substance, before coating with a controlled release coating film. Because lansoprazole is unstable to acid, an intermediate layer may be formed to prevent a direct contact between the pharmaceutically active ingredient-containing layer and the controlled release coating film, which is preferable for improving the stability of the pharmaceutically active ingredient. Such intermediate layer may be formed in a plurality of layers (preferably 2 or 3 layers).

Examples of the coating substance for an intermediate layer include a polymer substance such as L-HPC, hydroxypropylcellulose, HPMC (e.g., TC-5 etc.), polyvinylpyrrolidone, polyvinyl alcohol, methylcellulose, hydroxyethylmethylcellulose and the like, which is appropriately added with saccharides such as sucrose [purified sucrose (pulverized (powder sugar), non-pulverized) etc.], starch sugar such as cornstarch and the like, lactose, honey and sugar alcohol (D-mannitol, erythritol and the like) and the like. Preferred are L-HPC, HPMC, D-mannitol, and a mixture of these. Besides these, the intermediate layer may appropriately contain an excipient (e.g., masking agent (titanium oxide etc.), an antistatic agent (titanium oxide, talc etc.)) for the production of a preparation.

When the “fine granules containing a pharmaceutically active ingredient” have an intermediate layer, the amount of the intermediate layer is generally about 0.02 part by weight- about 1.5 parts by weight, preferably about 0.05- about 1 part by weight, per 1 part by weight of the granules before coating of an intermediate layer.

The thickness of the intermediate layer is not particularly limited as long as the effect of the present invention can be achieved. For example, the thickness is not less than 5 μm and not more than 50 μm, preferably not less than about 10 μm and not more than 40 μm.

The coating of the intermediate layer can be performed by a conventional method. For example, in a preferable method, the aforementioned intermediate layer component is diluted with purified water and the like, and sprayed as a liquid.

Such intermediate layer may be a plurality of layers (preferably 2 or 3 layers). When multiple layers are formed, they may have different compositions, and may be formed plural times in any order with other coating layers. For example, fine granules containing a pharmaceutically active ingredient are coated with a film of intermediate layer, followed by coating a controlled release coating film, followed by coating a film of intermediate layer again, followed by coating a controlled release coating film, followed by further coating a film containing polyethylene glycol.

More specifically, for example, fine granules containing a pharmaceutically active ingredient are coated with a film of intermediate layer, followed by coating a controlled release coating film containing methacrylic acid/methyl acrylate/methyl methacrylate copolymer, followed by coating a film of intermediate layer again, followed by coating a controlled release coating film containing methacrylic acid/methyl acrylate/methyl methacrylate copolymer, followed by further coating a film containing polyethylene glycol.

In the present invention, a basic inorganic compound is preferably added to the “fine granules” or the “fine granules containing a pharmaceutically active ingredient”, so as to stabilize lansoprazole in a formulation. The basic compound is preferably contacted with a pharmaceutically active ingredient, and preferably uniformly mixed with a pharmaceutically active ingredient.

Examples of the basic compound include basic inorganic salt, amino acid, and basic organic substance.

Examples of the “basic inorganic salt” include basic inorganic salts of sodium, potassium, magnesium and calcium (e.g., sodium carbonate, sodium hydrogen carbonate, potassium carbonate, potassium hydrogen carbonate, heavy magnesium carbonate, magnesium carbonate, magnesium oxide, magnesium hydroxide, calcium carbonate).

The amount of the basic inorganic salt to be used is appropriately determined according to the kind of the basic inorganic salt, and, for example, about 0.3- about 200 wt %, preferably about 1- about 100 wt %, more preferably about 10- about 50 wt %, most preferably about 20-40 wt %, of the pharmaceutically active ingredient.

Examples of the amino acid include arginine and lysine.

Examples of the basic organic substance include meglumine.

The “fine granules containing a pharmaceutically active ingredient” and “fine granules” in the present invention may be further coated with a diffusion-controlled release coating film, a water-soluble sugar alcohol, or an enteric coating film.

As the diffusion-controlling coating film, ethyl acrylate/methyl methacrylate copolymer, ethylcellulose, aminoacrylic methacrylate copolymer, polyvinyl acetate and the like can be mentioned, and two or more kinds thereof may be used in a mixture.

Examples of the water-soluble sugar alcohol include mannitol, sorbitol, maltitol, reduced starch saccharides, xylitol, reduced paratinose, erythritol. Preferred are mannitol, sorbitol, maltitol, xylitol and erythritol, more preferred are mannitol, sorbitol, maltitol and erythritol, and more preferred are mannitol and erythritol. When “fine granules” is coated, particularly overcoated, with water-soluble sugar alcohol, the strength of the orally disintegrating tablet containing the fine granules is improved.

Examples of the enteric coating film include polymers generally used as enteric coating films and, methacrylic acid/methyl acrylate/methyl methacrylate copolymer (Eudragit FS30D, manufactured by Evonik), methacrylic acid/ethyl acrylate copolymer (Eudragit L100-55 or Eudragit L30D-55, manufactured by Evonik), hypromellose phthalate (HP-55, HP-50, manufactured by Shin-Etsu Chemical Co., Ltd.), carboxymethylethylcellulose (CMEC, manufactured by Freund Corporation), polyvinyl acetate phthalate, hydroxypropyl methylcellulose acetate succinate (HPMCAS, manufactured by Shin-Etsu Chemical Co., Ltd.) and cellulose acetate phthalate and the like can be mentioned. They may be used alone and 2 or more kinds (preferably 2 to 4 kinds) may be mixed before use. Plural layers (e.g., 2-3 layers) may be formed.

The orally disintegrating tablet of the present invention can be produced in accordance with a conventional method in the pharmaceutical field.

Such methods include, for instance, a method which comprises blending the aforementioned two kinds of fine granules and the additives, and molding, if necessary followed by drying. Concretely mentioned is a method which comprises blending the fine granules and the additives, if necessary with water, and tableting, if necessary followed by drying.

The “blending procedure” can be carried out by any of the conventional blending techniques such as admixing, kneading and granulating. The above “blending procedure” is carried out, for instance, by using an apparatus such as Vertical granulator GV10 (manufactured by Powrex Corporation), Universal Kneader (manufactured by Hata Iron Works Co., Ltd.), fluidized bed coater LAB-1 and FD-3S, FD-WSG-60, MP-10 TUKU-2 type (manufactured by Powrex Corporation), V-shape mixer, and tumbling mixer.

The “molding” is performed by tableting by a single punch tablet machine (manufactured by Kikusui Seisakusho Ltd.), rotary tableting machine (manufactured by Kikusui Seisakusho Ltd.) and the like at a pressure of about 10- about 70 kN/cm², preferably about 20- about 60 kN/cm².

A production method by wet tableting is preferably the method described in JP-A-5-271054 and the like. They may also be produced by drying after humidifying. The method is preferably the method described in JP-A-9-48726, JP-A-8-291051 and the like. That is, it is effective to enhance hardness by humidifying before or after tableting and drying thereafter.

Raw material powders and granules may be punched at room temperature, or may be heat tableted at a temperature not lower than room temperature (about 25° C.- about 40° C.). In the present specification, the “room temperature” refers to the temperature in the room where tableting is performed in general tablet production, which is generally about 20° C.- about 25° C.

The “drying” may be performed by any method generally used for drying preparations, such as vacuum drying, fluidized bed drying and the like.

Tablet (I) and tablet (II) of the present invention optionally further contain an additive as a component other than “fine granules (i)” and “fine granules (ii)”.

As the additive to be blended with fine granules, for example, water-soluble sugar alcohol, crystalline cellulose or low-substituted hydroxypropylcellulose (hereinafter L-HPC) can be used. The orally disintegrating tablet can be produced by further adding and mixing a binder, an acidulant, an effervescent agent, an artificial sweetener, a flavor, a lubricant, a colorant, an excipient, a disintegrant, and the like, and then tableting the mixture.

The water-soluble sugar alcohol means a sugar alcohol which requires less than 30 ml of water for dissolution within about 30 minutes when 1 g of the sugar alcohol is added to water and then strongly shaken at 20° C. for 30 seconds every 5 minutes.

Examples of the “water-soluble sugar alcohol” include mannitol, sorbitol, maltitol, a hydrogenated starch hydrolysate, xylitol, reduced palatinose, erythritol, and the like. Preferable examples of the “water-soluble sugar alcohol” include mannitol, sorbitol, maltitol, xylitol, erythritol, more preferably mannitol, sorbitol, maltitol, erythritol, more preferably mannitol, erythritol can be mentioned. The water-soluble sugar alcohol may be a mixture of two or more kinds of them at an appropriate ratio. Erythritol is conventionally produced by fermentation of glucose as a raw material with yeast or the like. In the present invention, erythritol having a particle size of not more than 50 mesh is used. The erythritol is commercially available (Nikken Chemicals Co., Ltd., etc.). The amount of the “water-soluble sugar alcohol” is usually about 3 to about 50 parts by weight, preferably about 5 to about 40 parts by weight based on 100 parts by weight of a total formulation.

The “crystalline cellulose” may be obtained by partial depolymerization of α-cellulose followed by purification. The “crystalline cellulose” also includes microcrystalline cellulose. Specific examples of the crystalline cellulose include Ceolus KG-1000, Ceolus KG-802, CEOLUS PH-101, CEOLUS PH-102, CEOLUS PH-301, CEOLUS PH-302, CEOLUS UF-702, CEOLUS UF-711. Preferred are CEOLUS KG-802 and CEOLUS UF-711. These crystalline celluloses may be used alone or two or more kinds thereof may be used in combination. These crystalline celluloses are commercially available (Asahi Kasei Corporation). The crystalline cellulose may be incorporated in an amount of about 3 to about 50 parts by weight, preferably about 5 to about 40 parts by weight, most preferably about 5 to about 20 parts by weight into 100 parts by weight of a total formulation.

As the “low-substituted hydroxypropylcellulose”, LH-11, LH-21, LH-22, LH-B1, LH-31, LH-32, and LH-33 can be mentioned. The L-HPC can be obtained as commercially available products [manufactured by Shin-Etsu Chemical Co., Ltd.]. The low-substituted hydroxypropylcellulose can be added in a proportion of about 1- about 50 parts by weight, preferably about 3- about 40 parts by weight, most preferably, about 3- about 20 parts by weight, per 100 parts by weight of the whole formulation. The L-HPC having an HPC group content of 5.0-7.0 wt % or 7.0-9.9% to be used as an additive other than fine granules is added in a proportion of generally about 1- about 50 parts by weight, preferably about 1- about 40 parts by weight, more preferably about 1- about 20 parts by weight, per 100 parts by weight of the whole formulation, so as to obtain sufficient disintegration property in the oral cavity and sufficient formulation strength.

Examples of the binder include hydroxypropylcellulose, HPMC, crystalline cellulose, pregelatinized starch, polyvinylpyrrolidone, gum arabic powder, gelatin, pullulan, and the like. Two or more kinds of these binders may be used as a mixture at an appropriate ratio.

Examples of the acidulant include citric acid (anhydrous citric acid), tartaric acid, and malic acid.

Examples of the effervescent agent include sodium bicarbonate. Preferably, the preparation of the present invention does not contain an effervescent agent.

Examples of the artificial sweetener include saccharine sodium, dipotassium glycyrrhizinate, aspartame, sucralose, acesulfame-K, stevia, and thaumatin.

The flavor may be synthetic or natural, and examples thereof include lemon, lemon lime, orange, menthol, and strawberry.

Examples of the lubricant include magnesium stearate, a sucrose ester of fatty acid, polyethylene glycol, talc, and stearic acid.

Examples of the colorant include edible dyes such as food Yellow No. 5, food Red No. 2, and food Blue No. 2; an edible lake dye, ferric oxide and yellow ferric oxide.

Examples of the excipient include lactose, sucrose, D-mannitol (β-D-mannitol, etc.), starch, cornstarch, crystalline cellulose, light anhydrous silicic acid, titanium oxide and the like.

Examples of the disintegrant include crospovidone [manufactured by ISP Inc. (USA), or BASF (Germany)], croscarmellose sodium (FMC-Asahi Kasei Corporation), carmellose calcium (GOTOKU CHEMICAL COMPANY LTD.), low-substituted hydroxypropylcellulose, sodium carboxymethyl starch (Matsutani Chemical Industry Co., Ltd.), and cornstarch. Crospovidone is preferably used. Two or more kinds of these disintegrants may be used as a mixture at an appropriate ratio. For example, crospovidone may be used alone or in combination with other disintegrants. The crospovidone may be any crosslinked polymer referred to as 1-ethenyl-2-pyrrolidinone homopolymer, including polyvinyl polypyrrolidone (PVPP) and 1-vinyl-2-pyrrolidinone homopolymers, and usually, the crospovidone having a molecular weight of 1,000,000 or more is used. Specific examples of commercially available crospovidone include cross-linked povidone, Kollidon CL [manufactured by BASF (Germany)], Polyplasdone XL, Polyplasdone XL-10, INF-10 [manufactured by ISP Inc. (USA)], polyvinylpyrrolidone, PVPP, 1-vinyl-2-pyrrolidinone homopolymers and the like. Such disintegrant is used in an amount of for example, about 0.1 to about 20 parts by weight, preferably about 1 to about 15 parts by weight, further preferably about 2 to about 10 parts by weight based on 100 parts by weight of a total preparation.

The orally disintegrating tablet of the present invention has a diameter of about 6.5- about 20 mm, preferably about 8- about 14 mm, to facilitate handling for administration. In another embodiment, the orally disintegrating tablet of the present invention has a diameter of about 6.5- about 20 mm, preferably about 8- about 15 mm, to facilitate handling for administration.

The orally disintegrating tablet of the present invention has a total weight of not more than about 1000 mg, preferably about 300- about 900 mg, when it contains 30 mg of a pharmaceutically active ingredient.

The oral disintegration time (a time until a solid formulation is completely disintegrated with saliva alone in the oral cavity of a healthy adult man or woman) of the orally disintegrating tablet of the present invention is usually within about 90 seconds, preferably within about 1 minute, more preferably about 5 to about 50 seconds.

The disintegration time in water of the orally disintegrating tablet of the present invention is usually within about 90 seconds, preferably within about 1 minute.

The hardness (a value measured with a tablet hardness tester) of the orally disintegrating tablet of the present invention is usually about 10 N to about 150 N (about 1 kg to about 15 kg).

The orally disintegrating tablet of the present invention is administered without water or together with water. Examples of an administration method include (1) a method comprising putting the tablet of the present invention in the mouth and not swallowing the tablet, and then dissolving or disintegrating the tablet with a small amount of water or with saliva in the oral cavity without water and (2) a method comprising swallowing the tablet of the present invention together with water. Alternatively, the tablet of the present invention may be dissolved or disintegrated with water, and then be administered.

While the dose of the orally disintegrating tablet of the present invention varies depending on the severity of the symptoms, age, sex, body weight of the subject, timing and interval of administration, kind of the active ingredient and the like, it may be any as long as the dose of the pharmaceutically active ingredient is an effective amount. In addition, the orally disintegrating tablet of the present invention may be administered once a day or 2-3 portions a day.

The orally disintegrating tablet of the present invention is useful for treatment and prevention of a peptic ulcer (e.g., stomach ulcer, duodenal ulcer, anastomomic ulcer, Zollinger-Ellinson syndrome, etc.), gastritis, erosive esophagitis, symptomatic gastroesophageal reflex disease (symptomatic GERD) and the like; elimination or assistance in elimination of H. pylori; suppression of upper gastrointestinal tract bleeding caused by peptic ulcer, acute stress ulcer or hemorrhagic gastritis; suppression of upper gastrointestinal tract bleeding caused by invasive stress (stress caused by major operation which requires central control after operation, or cerebrovascular disorder, head trauma, multiple organ failure or extensive burn which requires intensive care); treatment and prevention of an ulcer caused by a non-steroidal antiinflammatory agent; treatment and prevention of gastric hyperacidity and an ulcer caused by postoperative stress; administration before anesthesia and the like. The dose of lansoprazole or optical isomers is about 0.5 to about 1500 mg/day, preferably about 5 to about 500 mg/day, more preferably about 5 to about 150 mg/day, for an adult (60 kg body weight).

The orally disintegrating tablet of the present invention can be orally administered to a mammal (e.g., human, monkey, sheep, horse, dog, cat, rabbit, rat, mouse and the like) for the treatment or prophylaxis of peptic ulcer (e.g., gastric ulcer, duodenal ulcer, anastomotic ulcer, Zollinger-Ellison syndrome etc.), gastritis, erosive esophagitis, symptomatic gastroesophageal reflux disease (symptomatic GERD) and the like; and the like.

Lansoprazole or an optically active form thereof may be used in combination with other medicaments (antitumor agent, antibacterial agent etc.). Particularly, a combined use with an antibacterial agent selected from erythromycin antibiotics (e.g., clarithromycin etc.), penicillin antibiotics (e.g., amoxicillin etc.) and imidazole compounds (e.g., metronidazole etc.) affords a superior effect for eradication of H. pylori.

In the orally disintegrating tablet of the present invention, desired is the controlled release formulation which can achieve an average pH in the stomach of not less than 4 in 0.5 hr and maintain the pH in the stomach of not less than 4 for 14 hours or longer.

The orally disintegrating tablet of the present invention is, for example, a formulation comprising R-lansoprazole or a salt thereof as a pharmaceutically active ingredient, which reaches the maximum blood drug concentration within about 5 to about 8 hours and maintains blood drug concentration of 100 ng/mL or above for about 4 hours or longer, when 30 mg of the pharmaceutically active ingredient is administered orally.

Since the orally disintegrating tablet of the present invention shows suppressed breakage of fine granules, the acid resistance of a medicament unstable to acid can be retained and the release of the pharmaceutically active ingredient can be controlled as desired. Since it contains two kinds of fine granules showing different release profiles of the pharmaceutically active ingredient, the release of the pharmaceutically active ingredient can be controlled for a long time. Therefore a therapeutically effective concentration can be maintained for a prolonged time, and administration frequency can be reduced, and effectiveness of treatment at a low dose and reduction of side effects caused by the rise of blood concentration can be ensured. In addition, since it shows superior disintegration property in the oral cavity, it is an orally disintegrating tablet which can be conveniently taken by elderly persons and children even without water. Furthermore, the orally disintegrating tablet of the present invention can suppress aggregation of fine granules during production, and contains fine granules with superior hardness. Therefore, it can also be applied to industrial large-scale production.

### EXAMPLES

The present invention is explained in more detail in the following by referring to Production Examples, Reference Examples, Examples, Comparative Examples and Experimental Examples, which are not to be construed as limitative.

The additives (e.g., mannitol, sucralose) used in the following Production Examples, Reference Examples, Examples and Comparative Examples were the Japanese Pharmacopoeia 15th Edition or Japanese Pharmaceutical Excipients 2003 compatible products. In the following Production Examples and Reference Examples, compound X is (R)-2-[[[3-methyl-4-(2,2,2-trifluoroethoxy)-2-pyridinyl]methyl]sulfinyl]-1H-benzimidazole. The properties of the fine granules, granules and formulations obtained in the Production Examples, Reference Examples, Examples and Comparative Examples were evaluated by the following test methods.

### (1) Dissolution Test

A dissolution test was performed by any of the following methods using basket method (USP Apparatus 1 method) or flow-through cell method (USP Apparatus 4 method). In the basket method, a dissolution tester manufactured by Toyama Sangyo CO., LTD. was used and, in the flow-through cell method, a dissolution tester manufactured by SOTAX was used. test method (1) basket method

After acid resistance test (0.1N HCl, 150 rpm, 500 mL, 1 h), a buffer test (50 mM phosphate buffer (pH 6.0) containing 5 mM Tween 20, 150 rpm, 900 mL) was performed. test method (2) basket method

After acid resistance test (0.1N HCl, 150 rpm, 500 mL, 1 h), a buffer test (50 mM phosphate buffer (pH 7.2) containing 5 mM Tween 20, 150 rpm, 900 mL) was performed. test method (3) flow-through cell method

After acid resistance test (0.1N HCl, 16 mL/min, 30 min), buffer test (1) (50 mM phosphate buffer (pH 6.0) containing 0.5 mM sodium dodecyl sulfate, 16 mL/min, 54 min), and buffer test (2) (50 mM phosphate buffer (pH 7.0) containing 0.5 mM sodium dodecyl sulfate, 16 mL/min) were successively performed.

### (2) Hardness Test

Tablet hardness was measured by using a tablet hardness tester (manufactured by Toyama Sangyo Co., Ltd.). The test was run 10 times and the average thereof is shown.

### (3) Disintegration Test in Oral Cavity

The time necessary for a tablet to be completely disintegrated in the oral cavity with saliva alone was measured. Three subjects performed the test and the average is thereof is shown.

### (4) Disintegration Test

The disintegration time was measured by a tablet disintegration tester (manufactured by Toyama Sangyo Co., Ltd.). The test was run 6 times and the average thereof is shown.

### Production Example 1

### Production of Fine Granules Containing a Pharmaceutically Active Ingredient

Hydroxypropyl cellulose (360 g) was dissolved in purified water (4680 g), and low-substituted hydroxypropyl cellulose (L-HPC-32, 180 g) and magnesium carbonate (360 g) were dispersed in this solution. Compound X (1080 g) was uniformly dispersed in the obtained dispersion to give a coating solution. Lactose/crystalline cellulose spheres (Nonpareil 105T, 900 g) were coated with a predetermined amount (5550 g) from the compound X-containing coating solution (6660 g) by using a tumbling fluidized bed coater (MP-10 TOKU-2 type, manufactured by POWREX Corporation). The coating conditions were: inlet air temperature about 85° C., spray air pressure about 0.25 MPa, spray air volume about 80 Nl/min, inlet air volume about 0.7 m³/min, rotor rev rate about 500 rpm, spray rate about 15 g/min, spray position lower side.

[Composition of fine granules containing a
pharmaceutically active ingredient (85 mg)]

| | |
|---|---|
| lactose/crystalline cellulose spheres | 30 mg |
| (Nonpareil 105T) | |
| compound X | 30 mg |
| magnesium carbonate | 10 mg |
| low-substituted hydroxypropyl cellulose | 5 mg |
| hydroxypropyl cellulose | 10 mg |
| total | 85 mg |

### Production Example 2

### Production of Fine Granules Coated with Intermediate Layer

The fine granules containing a pharmaceutically active ingredient obtained in Production Example 1 were coated with an intermediate layer coating solution by using a tumbling fluidized bed coater (MP-10 TOKU-2 type, manufactured by POWREX CORPORATION), and then dried to give fine granules with the following composition. The intermediate layer coating solution was produced by dissolving hypromellose (TC-5E, 252 g) and mannitol (252 g) in purified water (2700 g), and dispersing titanium oxide (108 g), talc (108 g) and low-substituted hydroxypropyl cellulose (L-HPC-32, 180 g) in the obtained solution. The fine granules containing a pharmaceutically active ingredient (2550 g) obtained in Production Example 1 were coated with a predetermined amount (3000 g) of the intermediate layer coating solution (3600 g) by using a tumbling fluidized bed coater (MP-10 TOKU-2 type, manufactured by POWREX CORPORATION). The coating conditions were: inlet air temperature about 85° C., spray air pressure about 0.35 MPa, spray air volume about 100 Nl/min, inlet air volume about 1.5 m³/min, rotor rev rate about 550 rpm, spray rate about 18 g/min, spray position lower side. After the completion of coating, the obtained fine granules were then dried at 85° C. for about 40 min in the tumbling fluidized bed coater and passed through a round sieve to give fine granules coated with intermediate layer with a particle size of 150 μm-350 μm.

[Composition of fine granules coated with
intermediate layer (110 mg)]

| | |
|---|---|
| fine granules containing a pharmaceutically | 85 mg |
| active ingredient (Production Example 1) | |
| hypromellose | 7 mg |
| low-substituted hydroxypropyl cellulose | 5 mg |
| talc | 3 mg |
| titanium oxide | 3 mg |
| mannitol | 7 mg |
| total | 110 mg |

### Production Example 3

### Production of Fine Granules Coated with Intermediate Layer

The fine granules containing a pharmaceutically active ingredient obtained in Production Example 1 was coated with an intermediate layer coating solution by using a tumbling fluidized bed coater (MP-10 TOKU-2 type, manufactured by POWREX CORPORATION), and then dried to give fine granules with the following composition. The intermediate layer coating solution was produced by dissolving hypromellose (TC-5E, 504 g) and mannitol (504 g) in purified water (5400 g), and dispersing titanium oxide (216 g), talc (216 g) and low-substituted hydroxypropyl cellulose (L-HPC-32, 360 g) in the obtained solution. The fine granules containing a pharmaceutically active ingredient (2550 g) obtained in Production Example 1 were coated with a predetermined amount (6000 g) of the intermediate layer coating solution (7200 g) by using a tumbling fluidized bed coater (MP-10 TOKU-2 type, manufactured by POWREX CORPORATION). The coating conditions were: inlet air temperature about 85° C., spray air pressure about 0.35 MPa, spray air volume about 100 Nl/min, inlet air volume about 1.5 m³/min, rotor rev rate about 550 rpm, spray rate about 18 g/min, spray position lower side. After the completion of coating, the obtained fine granules were then dried at 85° C. for about 40 min in the tumbling fluidized bed coater and passed through a round sieve to give fine granules coated with intermediate layer with a particle size of 150 μm-350 μm.

[Composition of fine granules coated with
intermediate layer (110 mg)]

| | |
|---|---|
| fine granules containing a pharmaceutically | 85 mg |
| active ingredient (Production Example 1) | |
| hypromellose | 14 mg |
| low-substituted hydroxypropyl cellulose | 10 mg |
| talc | 6 mg |
| titanium oxide | 6 mg |
| mannitol | 14 mg |
| total | 135 mg |

### Production Example 4

### Production of Controlled Release Fine Granules

Purified water (3474.5 g) was heated to 80° C., and polysorbate 80 (29.55 g), glycerol monostearate (73.87 g), triethyl citrate (246.5 g), yellow ferric oxide (2.373 g) and ferric oxide (2.373 g) were dispersed therein. The suspension was cooled to room temperature, and then ethyl acrylate/methyl methacrylate copolymer dispersion (Eudragit NE30D) (410.4 g) and citric acid (1.231 g) were added and the mixture was uniformly mixed. Furthermore, methacrylic acid/ethyl acrylate copolymer dispersion (Eudragit L30D-55) (3694 g) was added and the mixture was uniformly mixed to give a coating solution. The fine granules coated with intermediate layer (1282.5 g) obtained in Production Example 3 were coated with a predetermined amount (6942 g, 5% increased charge amount) of the aforementioned coating solution (7934 g) by using a tumbling fluidized bed coater (MP-10 TOKU-2 type, manufactured by POWREX CORPORATION). The coating conditions were: inlet air temperature 80° C., spray air pressure about 0.45 MPa, spray air volume about 120 Nl/min, inlet air volume about 1.5 m³/min, rotor rev rate about 600 rpm, spray rate about 19 g/min, spray position lower side.

[Composition of controlled release fine granules (68.549 mg)]

| | | |
|---|---|---|
| fine granules coated with intermediate layer | 33.75 | mg |
| (Production Example 3) | | |
| methacrylic acid/ethyl acrylate copolymer | 24.3 | mg |
| ethyl acrylate/methyl methacrylate copolymer | 2.7 | mg |
| polysorbate 80 | 0.648 | mg |
| glycerol monostearate | 1.62 | mg |
| triethyl citrate | 5.4 | mg |
| citric acid | 0.027 | mg |
| yellow ferric oxide | 0.052 | mg |
| ferric oxide | 0.052 | mg |
| total | 68.549 | mg |

### Production Example 5

### Production of Controlled Release Fine Granules

Purified water (4343.3 g) was heated to 80° C., and polysorbate 80 (36.94 g), glycerol monostearate (92.34 g), triethyl citrate (307.8 g), yellow ferric oxide (2.966 g) and ferric oxide (2.966 g) were dispersed therein. The suspension was cooled to room temperature, and then ethyl acrylate/methyl methacrylate copolymer dispersion (Eudragit NE30D) (513 g) and citric acid (1.539 g) were added and the mixture was uniformly mixed. Furthermore, methacrylic acid/ethyl acrylate copolymer dispersion (Eudragit L30D-55) (4617 g) was added and the mixture was uniformly mixed to give a coating solution. The fine granules coated with intermediate layer (1282.5 g) obtained in Production Example 3 were coated with a predetermined amount (8678 g, 5% increased charge amount) of the aforementioned coating solution (9918 g) by using a tumbling fluidized bed coater (MP-10 TOKU-2 type, manufactured by POWREX CORPORATION). The coating conditions were: inlet air temperature 80° C., spray air pressure about 0.45 MPa, spray air volume about 120 Nl/min, inlet air volume about 1.5 m³/min, rotor rev rate about 600 rpm, spray rate about 19 g/min, spray position lower side.

[Composition of controlled release fine granules (77.25 mg)]

| | | |
|---|---|---|
| fine granules coated with intermediate layer | 33.75 | mg |
| (Production Example 3) | | |
| methacrylic acid/ethyl acrylate copolymer | 30.375 | mg |
| ethyl acrylate/methyl methacrylate copolymer | 3.375 | mg |
| polysorbate 80 | 0.81 | mg |
| glycerol monostearate | 2.025 | mg |
| triethyl citrate | 6.75 | mg |
| citric acid | 0.03375 | mg |
| yellow ferric oxide | 0.065 | mg |
| ferric oxide | 0.065 | mg |
| total | 77.25 | mg |

### Production Example 6

### Production of Controlled Release Fine Granules

Purified water (5212.2 g) was heated to 80° C., and polysorbate 80 (44.32 g), glycerol monostearate (110.81 g), triethyl citrate (369.4 g), yellow ferric oxide (3.56 g) and ferric oxide (3.56 g) were dispersed therein. The suspension was cooled to room temperature, and then ethyl acrylate/methyl methacrylate copolymer dispersion (Eudragit NE30D) (615.6 g) and citric acid (1.847 g) were added and the mixture was uniformly mixed. Furthermore, methacrylic acid/ethyl acrylate copolymer dispersion (Eudragit L30D-55) (5540 g) was added and the mixture was uniformly mixed to give a coating solution. The fine granules coated with intermediate layer (1282.5 g) obtained in Production Example 3 were coated with a predetermined amount (10414 g, 5% increased charge amount) of the aforementioned coating solution (11901 g) by using a tumbling fluidized bed coater (MP-10 TOKU-2 type, manufactured by POWREX CORPORATION). The coating conditions were: inlet air temperature 80° C., spray air pressure about 0.45 MPa, spray air volume about 120 Nl/min, inlet air volume about 1.5 m³/min, rotor rev rate about 600 rpm, spray rate about 19 g/min, spray position lower side.

[Composition of controlled release fine granules (85.95 mg)]

| | | |
|---|---|---|
| fine granules coated with intermediate layer | 33.75 | mg |
| (Production Example 3) | | |
| methacrylic acid/ethyl acrylate copolymer | 36.45 | mg |
| ethyl acrylate/methyl methacrylate copolymer | 4.05 | mg |
| polysorbate 80 | 0.972 | mg |
| glycerol monostearate | 2.43 | mg |
| triethyl citrate | 8.1 | mg |
| citric acid | 0.0405 | mg |
| yellow ferric oxide | 0.078 | mg |
| ferric oxide | 0.078 | mg |
| total | 85.95 | mg |

### Production Example 7

### Production of Controlled Release Fine Granules

Purified water (6080 g) was heated to 80° C., and polysorbate 80 (51.71 g), glycerol monostearate (129.28 g), triethyl citrate (430.9 g), yellow ferric oxide (4.153 g) and ferric oxide (4.153 g) were dispersed therein. The suspension was cooled to room temperature, and then ethyl acrylate/methyl methacrylate copolymer dispersion (Eudragit NE30D) (718.2 g) and citric acid (2.155 g) were added and the mixture was uniformly mixed. Furthermore, methacrylic acid/ethyl acrylate copolymer dispersion (Eudragit L30D-55) (6464 g) was added and the mixture was uniformly mixed to give a coating solution. The fine granules coated with intermediate layer (1282.5 g) obtained in Production Example 3 were coated with a predetermined amount (12149 g, 5% increased charge amount) of the aforementioned coating solution (13885 g) by using a tumbling fluidized bed coater (MP-10 TOKU-2 type, manufactured by POWREX CORPORATION). The coating conditions were: inlet air temperature 80° C., spray air pressure about 0.45 MPa, spray air volume about 120 Nl/min, inlet air volume about 1.5 m³/min, rotor rev rate about 600 rpm, spray rate about 19 g/min, spray position lower side.

[Composition of controlled release fine granules (94.65 mg)]

| | | |
|---|---|---|
| fine granules coated with intermediate layer | 33.75 | mg |
| (Production Example 3) | | |
| methacrylic acid/ethyl acrylate copolymer | 42.525 | mg |
| ethyl acrylate/methyl methacrylate copolymer | 4.725 | mg |
| polysorbate 80 | 1.134 | mg |
| glycerol monostearate | 2.835 | mg |
| triethyl citrate | 9.45 | mg |
| citric acid | 0.04725 | mg |
| yellow ferric oxide | 0.091 | mg |
| ferric oxide | 0.091 | mg |
| total | 94.65 | mg |

### Production Example 8

### Production of Mannitol-Overcoated Controlled Release Fine Granules

Mannitol (190 g) was dissolved in purified water (1140 g) to give a coating solution. The controlled release fine granules (2935.5 g) obtained in Production Example 5 were coated with a predetermined amount (798 g, 5% increased charge amount) of the aforementioned coating solution (1330 g) by using a tumbling fluidized bed coater (MP-10 TOKU-2 type, manufactured by POWREX CORPORATION). The coating conditions for mannitol overcoating were: inlet air temperature about 80° C., spray air pressure about 0.45 MPa, spray air volume about 120 Nl/min, inlet air volume about 1.5 m³/min, rotor rev rate about 600 rpm, spray rate about 17 g/min, spray position lower side. The obtained fine granules were then dried at 85° C. for 40 min in the tumbling fluidized bed coater and passed through a round sieve to give the outermost layer-coated fine granules with a particle size of 250 μm-425 μm.

[Composition of mannitol-overcoated controlled release fine granules
(80.25 mg)]

| | | |
|---|---|---|
| controlled release fine granules | 77.25 | mg |
| (Production Example 5) | | |
| mannitol | 3.0 | mg |
| total | 80.25 | mg |

### Production Example 9

### Production of Mannitol-Overcoated Controlled Release Fine Granules

Mannitol (190 g) was dissolved in purified water (1140 g) to give a coating solution. The controlled release fine granules (3596.6 g) obtained in Production Example 7 were coated with a predetermined amount (798 g) of the aforementioned coating solution (1330 g) by using a tumbling fluidized bed coater (MP-10 TOKU-2 type, manufactured by POWREX CORPORATION, 5% increased charge amount). The coating conditions for mannitol overcoating were: inlet air temperature about 80° C., spray air pressure about 0.45 MPa, spray air volume about 120 Nl/min, inlet air volume about 1.5 m³/min, rotor rev rate about 600 rpm, spray rate about 17 g/min, spray position lower side. The obtained fine granules were then dried at 85° C. for 40 min in the tumbling fluidized bed coater and passed through a round sieve to give the outermost layer-coated fine granules with a particle size of 250 μm-425 μm.

[Composition of mannitol-overcoated controlled release fine granules
(97.65 mg)]

| | | |
|---|---|---|
| controlled release fine granules | 94.65 | mg |
| (Production Example 7) | | |
| mannitol | 3.0 | mg |
| total | 97.65 | mg |

### Production Example 10

### Production of Controlled Release Fine Granules

Purified water (275.78 g) was heated to 80° C., and polysorbate 80 (2.734 g), glycerol monostearate (6.834 g), polyethylene glycol (11.39 g), yellow ferric oxide (0.2025 g) and ferric oxide (0.2025 g) were dispersed therein. The suspension was cooled to room temperature, and then ethyl acrylate/methyl methacrylate copolymer dispersion (Eudragit NE30D) (37.97 g) and citric acid (0.1139 g) were added and the mixture was uniformly mixed. Furthermore, methacrylic acid/ethyl acrylate copolymer dispersion (Eudragit L30D-55) (341.7 g) was added and the mixture was uniformly mixed to give a coating solution. The controlled release fine granules (2317.5 g) obtained in Production Example 5 were coated with a predetermined amount (474 g, 5% increased charge amount) of the aforementioned coating solution (677 g) by using a tumbling fluidized bed coater (MP-10 TOKU-2 type, manufactured by POWREX CORPORATION). The coating conditions were: inlet air temperature 80° C., spray air pressure about 0.45 MPa, spray air volume about 120 Nl/min, inlet air volume about 1.5 m³/min, rotor rev rate about 600 rpm, spray rate about 19 g/min, spray position lower side.

[Composition of controlled release fine granules (80.26 mg)]

| | | |
|---|---|---|
| controlled release fine granules | 77.25 | mg |
| (Production Example 5) | | |
| methacrylic acid/ethyl acrylate copolymer | 2.2781 | mg |
| ethyl acrylate/methyl methacrylate copolymer | 0.2531 | mg |
| polysorbate 80 | 0.0608 | mg |
| glycerol monostearate | 0.1519 | mg |
| polyethylene glycol | 0.2531 | mg |
| citric acid | 0.0025 | mg |
| yellow ferric oxide | 0.0045 | mg |
| ferric oxide | 0.0045 | mg |
| total | 80.26 | mg |

### Production Example 11

### Production of Mannitol-Overcoated Controlled Release Fine Granules

Mannitol (150 g) was dissolved in purified water (900 g) to give a coating solution. The controlled release fine granules (2407.8 g) obtained in Production Example 10 are coated with a predetermined amount (630 g, 5% increased charge amount) of the aforementioned coating solution (1050 g) by using a tumbling fluidized bed coater (MP-10 TOKU-2 type, manufactured by POWREX CORPORATION). The coating conditions for mannitol overcoating were: inlet air temperature 80° C., spray air pressure about 0.45 MPa, spray air volume about 120 Nl/min, inlet air volume about 1.5 m³/min, rotor rev rate about 600 rpm, spray rate about 17 g/min, spray position lower side. The obtained fine granules were then dried at 85° C. for 40 min in the tumbling fluidized bed coater and passed through a round sieve to give the outermost layer-coated fine granules with a particle size of 250 μm-425 μm.

[Composition of mannitol-overcoated controlled release fine granules
(83.26 mg)]

| | | |
|---|---|---|
| controlled release fine granules | 80.26 | mg |
| (Production Example 10) | | |
| mannitol | 3.0 | mg |
| total | 83.26 | mg |

### Reference Example 1

### Production of Controlled Release Fine Granules

Purified water (677.6 g) was heated to 80° C., and polysorbate 80 (5.775 g), glycerol monostearate (14.44 g) and triethyl citrate (28.875 g) were dispersed therein. The suspension was cooled to room temperature, and added to methacrylic acid/methyl acrylate/methyl methacrylate copolymer dispersion (Eudragit FS30D) (962.5 g) and uniformly mixed to give a coating solution. The fine granules coated with intermediate layer (577.5 g) obtained in Production Example 2 were coated with a predetermined amount (1013.5 g) of the aforementioned coating solution (1689.2 g) by using a tumbling fluidized bed coater (MP-01, manufactured by POWREX CORPORATION). The coating conditions were: inlet air temperature about 40° C., spray air pressure about 0.2 MPa, spray air volume about 90 Nl/min, inlet air volume about 0.5 m³/min, rotor rev rate about 500 rpm, spray rate about 4 g/min, spray position lower side.

[Composition of controlled release fine granules (111.4575 mg)]

| | | |
|---|---|---|
| fine granules coated with intermediate layer | 82.5 | mg |
| (Production Example 2) | | |
| methacrylic acid/methyl acrylate/methyl | 24.75 | mg |
| methacrylate copolymer | | |
| polysorbate 80 | 0.495 | mg |
| glycerol monostearate | 1.2375 | mg |
| triethyl citrate | 2.475 | mg |
| total | 111.4575 | mg |

### Reference Example 2

### Production of Controlled Release Fine Granules

Purified water (677.6 g) was heated to 80° C., and polysorbate 80 (5.775 g), glycerol monostearate (14.44 g) and triethyl citrate (28.875 g) were dispersed therein. The suspension was cooled to room temperature, and added to methacrylic acid/methyl acrylate/methyl methacrylate copolymer dispersion (Eudragit FS30D) (962.5 g) and uniformly mixed to give a coating solution. The fine granules coated with intermediate layer (399.85 g) obtained in Production Example 2 were coated with a predetermined amount (1481.3 g) of the aforementioned coating solution (1689.2 g) by using a tumbling fluidized bed coater (MP-01, manufactured by POWREX CORPORATION). The coating conditions were: inlet air temperature 40° C., spray air pressure about 0.2 MPa, spray air volume about 90 Nl/min, inlet air volume about 0.5 m³/min, rotor rev rate about 500 rpm, spray rate about 4 g/min, spray position lower side.

[Composition of controlled release fine granules (130.7625 mg)]

| | | |
|---|---|---|
| fine granules coated with intermediate layer | 82.5 | mg |
| (Production Example 2) | | |
| methacrylic acid/methyl acrylate/methyl | 41.25 | mg |
| methacrylate copolymer | | |
| polysorbate 80 | 0.825 | mg |
| glycerol monostearate | 2.0625 | mg |
| triethyl citrate | 4.125 | mg |
| total | 130.7625 | mg |

### Reference Example 3

### Production of Controlled Release Fine Granules

Purified water (216.83 g) was heated to 80° C., and polysorbate 80 (1.848 g), glycerol monostearate (4.62 g) and triethyl citrate (9.24 g) were dispersed therein. The suspension was cooled to room temperature, and added to methacrylic acid/methyl acrylate/methyl methacrylate copolymer dispersion (Eudragit FS30D) (308.0 g) and uniformly mixed to give a coating solution. The controlled release fine granules (209.22 g) obtained in Reference Example 2 were coated with a predetermined amount (231.66 g) of the aforementioned coating solution (540.54 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 300 rpm, spray rate about 2.0 g/min, spray position lower side.

[Composition of controlled release fine granules (159.72 mg)]

| | | |
|---|---|---|
| controlled release fine granules | 130.7625 | mg |
| (Reference Example 2) | | |
| methacrylic acid/methyl acrylate/methyl | 24.75 | mg |
| methacrylate copolymer | | |
| polysorbate 80 | 0.495 | mg |
| glycerol monostearate | 1.2375 | mg |
| triethyl citrate | 2.475 | mg |
| total | 159.72 | mg |

### Production Example 12

### Production of Controlled Release Fine Granules

Purified water (216.83 g) was heated to 80° C., and polysorbate 80 (1.848 g), glycerol monostearate (4.62 g) and triethyl citrate (9.24 g) were dispersed therein. The suspension was cooled to room temperature, and added to methacrylic acid/methyl acrylate/methyl methacrylate copolymer dispersion (Eudragit FS30D) (308.0 g) and uniformly mixed to give a coating solution. The controlled release fine granules (192.85 g) obtained in Reference Example 2 were coated with a predetermined amount (374.0 g) of the aforementioned coating solution (540.54 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 300 rpm, spray rate about 2.0 g/min, spray position lower side.

The thickness of the controlled release film of the obtained fine granules was about 38.6 μm.

[Composition of controlled release fine granules (179.025 mg)]

| | | |
|---|---|---|
| controlled release fine granules | 130.7625 | mg |
| (Reference Example 2) | | |
| methacrylic acid/methyl acrylate/methyl | 41.25 | mg |
| methacrylate copolymer | | |
| polysorbate 80 | 0.825 | mg |
| glycerol monostearate | 2.0625 | mg |
| triethyl citrate | 4.125 | mg |
| total | 179.025 | mg |

### Production Example 13

### Production of Controlled Release Fine Granules

Purified water (216.83 g) was heated to 80° C., and polysorbate 80 (1.848 g), glycerol monostearate (4.62 g) and triethyl citrate (9.24 g) were dispersed therein. The suspension was cooled to room temperature, and added to methacrylic acid/methyl acrylate/methyl methacrylate copolymer dispersion (Eudragit FS30D) (308.0 g) and uniformly mixed to give a coating solution. The controlled release fine granules (178.24 g) obtained in Reference Example 2 were coated with a predetermined amount (505.58 g) of the aforementioned coating solution (540.54 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 300 rpm, spray rate about 2.0 g/min, spray position lower side.

The thickness of the controlled release film of the obtained fine granules was about 44.5 μm.

[Composition of controlled release fine granules (198.33 mg)]

| | | |
|---|---|---|
| controlled release fine granules | 130.7625 | mg |
| (Reference Example 2) | | |
| methacrylic acid/methyl acrylate/methyl | 57.75 | mg |
| methacrylate copolymer | | |
| polysorbate 80 | 1.155 | mg |
| glycerol monostearate | 2.8875 | mg |
| triethyl citrate | 5.775 | mg |
| total | 198.33 | mg |

### Production Example 14

### Production of Mannitol-Overcoated Controlled Release Fine Granules

Mannitol (13.6 g) was dissolved in purified water (77.1 g) to give a coating solution. The controlled release fine granules (317.328 g) obtained in Production Example 13 were coated with the aforementioned coating solution (90.7 g) by using a tumbling fluidized bed coater (SPIR-A-FLOW, manufactured by Freund Corporation). The coating conditions for mannitol overcoating were: inlet air temperature about 45° C., spray air pressure about 0.2 MPa, spray air volume about 30 Nl/min, BED pressure about 1.4 MPa, rotor rev rate about 300 rpm, spray rate about 3.0 g/min, spray position lower side. The obtained fine granules were then dried at 50° C. for 40 min in the tumbling fluidized bed coater and passed through a round sieve to give the outermost layer-coated fine granules with a particle size of 250 μm-425 μm.

[Composition of mannitol-overcoated controlled release fine granules
(206.83 mg)]

| | | |
|---|---|---|
| controlled release fine granules | 198.33 | mg |
| (Production Example 13) | | |
| mannitol | 8.5 | mg |
| total | 206.83 | mg |

### Production Example 15

### Production of Controlled Release Fine Granules

Purified water (2297.7 g) was heated to 80° C., and polysorbate 80 (19.46 g), glycerol monostearate (48.66 g), triethyl citrate (97.32 g), yellow ferric oxide (1.708 g) and ferric oxide (1.708 g) were dispersed therein. The suspension was cooled to room temperature, and added to methacrylic acid/methyl acrylate/methyl methacrylate copolymer dispersion (Eudragit FS30D) (3244 g) and uniformly mixed to give a coating solution. The fine granules coated with intermediate layer (901.1 g) obtained in Production Example 3 were coated with a predetermined amount (4997 g, 5% increased charge amount) of the aforementioned coating solution (5710 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 38.1 μm.

[Composition of controlled release fine granules (208.19 mg)]

| | | |
|---|---|---|
| fine granules coated with intermediate layer | 101.25 | mg |
| (Production Example 3) | | |
| methacrylic acid/methyl acrylate/methyl | 91.125 | mg |
| methacrylate copolymer | | |
| polysorbate 80 | 1.8225 | mg |
| glycerol monostearate | 4.55625 | mg |
| triethyl citrate | 9.1125 | mg |
| yellow ferric oxide | 0.1599 | mg |
| ferric oxide | 0.1599 | mg |
| total | 208.19 | mg |

### Production Example 16

### Production of Mannitol-Overcoated Controlled Release Fine Granules

Mannitol (133.5 g) was dissolved in purified water (801 g) to give a coating solution. The controlled release fine granules (1852.8 g) obtained in Production Example 15 were coated with a predetermined amount (560.7 g, 5% increased charge amount) of the aforementioned coating solution (934.5 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.2 m³/min, rotor rev rate about 550 rpm, spray rate about 10 g/min, spray position lower side. The obtained fine granules were then dried at 85° C. for about 40 min in the tumbling fluidized bed coater and passed through a round sieve to give the outermost layer-coated fine granules with a particle size of 250 μm-425 μm.

[Composition of mannitol-overcoated controlled release fine granules
(217.2 mg)]

| | | |
|---|---|---|
| controlled release fine granules | 208.19 | mg |
| (Production Example 15) | | |
| mannitol | 9.0 | mg |
| total | 217.2 | mg |

### Production Example 17

### Production of Controlled Release Fine Granules

Purified water (2807.8 g) was heated to 80° C., and polysorbate 80 (23.79 g), glycerol monostearate (59.47 g), triethyl citrate (118.9 g), yellow ferric oxide (2.088 g) and ferric oxide (2.088 g) were dispersed therein. The suspension was cooled to room temperature, and added to methacrylic acid/methyl acrylate/methyl methacrylate copolymer dispersion (Eudragit FS30D) (3965 g) and uniformly mixed to give a coating solution. The fine granules coated with intermediate layer (901.1 g), which were obtained in Production Example 3 were coated with a predetermined amount (6107 g, 5% increased charge amount) of the aforementioned coating solution (6979 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 9 g/min, spray position lower side.

The thickness of the controlled release film of the obtained fine granules was about 44.7 μm.

[Composition of controlled release fine granules (231.95 mg)]

| | | |
|---|---|---|
| fine granules coated with intermediate layer | 101.25 | mg |
| (Production Example 3) | | |
| methacrylic acid/methyl acrylate/methyl | 111.375 | mg |
| methacrylate copolymer | | |
| polysorbate 80 | 2.228 | mg |
| glycerol monostearate | 5.569 | mg |
| triethyl citrate | 11.138 | mg |
| yellow ferric oxide | 0.195 | mg |
| ferric oxide | 0.195 | mg |
| total | 231.95 | mg |

### Production Example 18

### Production of Mannitol-Overcoated Controlled Release Fine Granules

Mannitol (140.9 g) was dissolved in purified water (845.5 g) to give a coating solution. The controlled release fine granules (2064.3 g) obtained in Production Example 17 were coated with a predetermined amount (591.9 g, 5% increased charge amount) of the aforementioned coating solution (986.4 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.2 m³/min, rotor rev rate about 550 rpm, spray rate about 10 g/min, spray position lower side. The obtained fine granules were then dried at 85° C. for 40 min in the tumbling fluidized bed coater and passed through a round sieve to give the outermost layer-coated fine granules with a particle size of 250 μm-425 μm.

[Composition of mannitol-overcoated controlled release fine granules
(241.45 mg)]

| | | |
|---|---|---|
| controlled release fine granules | 231.95 | mg |
| (Production Example 17) | | |
| mannitol | 9.5 | mg |
| total | 241.45 | mg |

### Production Example 19

### Production of Controlled Release Fine Granules

Purified water (435.22 g) was heated to 80° C., and polysorbate 80 (4.315 g), glycerol monostearate (10.786 g), polyethylene glycol (17.98 g), yellow ferric oxide (0.3195 g) and ferric oxide (0.3195 g) were dispersed therein. The suspension was cooled to room temperature, and then ethyl acrylate/methyl methacrylate copolymer dispersion (Eudragit NE30D) (59.92 g) and citric acid (0.1798 g) were added and the mixture was uniformly mixed. Furthermore, methacrylic acid/ethyl acrylate copolymer dispersion (Eudragit L30D-55) (539.3 g) was added and the mixture was uniformly mixed to give a coating solution. The controlled release fine granules (1852.8 g) obtained in Production Example 15 were coated with a predetermined amount (374 g, 5% increased charge amount) of the aforementioned coating solution (1068 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.5 m³/min, rotor rev rate about 600 rpm, spray rate about 10 g/min, spray position lower side.

[Composition of controlled release fine granules (216.19 mg)]

| | | |
|---|---|---|
| controlled release fine granules | 208.19 | mg |
| (Production Example 15) | | |
| methacrylic acid/ethyl acrylate copolymer | 6.06 | mg |
| ethyl acrylate/methyl methacrylate copolymer | 0.673 | mg |
| polysorbate 80 | 0.162 | mg |
| glycerol monostearate | 0.404 | mg |
| polyethylene glycol | 0.673 | mg |
| citric acid | 0.007 | mg |
| yellow ferric oxide | 0.012 | mg |
| ferric oxide | 0.012 | mg |
| total | 216.19 | mg |

### Production Example 20

### Production of Mannitol-Overcoated Controlled Release Fine Granules

Mannitol (118.7 g) was dissolved in purified water (712 g) to give a coating solution. The controlled release fine granules (1924 g) obtained in Production Example 19 were coated with a predetermined amount (498.4 g, 5% increased charge amount) of the aforementioned coating solution (830.7 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.5 m³/min, rotor rev rate about 600 rpm, spray rate about 12 g/min, spray position lower side. The obtained fine granules were then dried at 85° C. for 40 min in the tumbling fluidized bed coater and passed through a round sieve to give the outermost layer-coated fine granules with a particle size of 250 μm-425 μm.

[Composition of mannitol-overcoated controlled release fine
granules (224.19 mg)]

| | | |
|---|---|---|
| controlled release fine granules | 216.19 | mg |
| (Production Example 19) | | |
| mannitol | 8 | mg |
| total | 224.19 | mg |

### Production Example 21

### Production of Controlled Release Fine Granules

Purified water (482.625 g) was heated to 80° C., and polysorbate 80 (4.455 g), glycerol monostearate (11.1375 g) and triethyl citrate (11.1375 g) were dispersed therein. The suspension was cooled to room temperature, and then ethyl acrylate/methyl methacrylate copolymer dispersion (Eudragit NE30D) (371.25 g) and citric acid (1.1138 g) were added and the mixture was uniformly mixed. Furthermore, methacrylic acid/methyl acrylate/methyl methacrylate copolymer dispersion (Eudragit FS30D) (371.25 g) was added and the mixture was uniformly mixed to give a coating solution. The fine granules coated with intermediate layer (742.5 g) obtained in Production Example 2 were coated with the aforementioned coating solution (1252.97 g) by using a tumbling fluidized bed coater (MP-01, manufactured by POWREX CORPORATION). The coating conditions were: inlet air temperature 30° C., spray air pressure about 0.2 MPa, spray air volume about 90 Nl/min, inlet air volume about 0.5 m³/min, rotor rev rate about 500 rpm, spray rate about 4 g/min, spray position lower side.

[Composition of controlled release fine granules (110.34375 mg)]

| | | |
|---|---|---|
| fine granules coated with intermediate layer | 82.5 | mg |
| (Production Example 2) | | |
| methacrylic acid/methyl acrylate/methyl | 12.375 | mg |
| methacrylate copolymer | | |
| ethyl acrylate/methyl methacrylate copolymer | 12.375 | mg |
| polysorbate 80 | 0.495 | mg |
| glycerol monostearate | 1.2375 | mg |
| triethyl citrate | 1.2375 | mg |
| citric acid | 0.12375 | mg |
| total | 110.34375 | mg |

### Production Example 22

### Production of Controlled Release Fine Granules

Purified water (248.18 g) was heated to 80° C., and polysorbate 80 (2.1152 g), glycerol monostearate (5.288 g) and triethyl citrate (10.576 g) were dispersed therein. The suspension was cooled to room temperature, and added to methacrylic acid/methyl acrylate/methyl methacrylate copolymer dispersion (Eudragit FS30D) (352.5333 g) and uniformly mixed to give a coating solution. The controlled release fine granules (220.6875 g) obtained in Production Example 21 were coated with the aforementioned coating solution (618.696 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 300 rpm, spray rate about 3.0 g/min, spray position lower side.

[Composition of controlled release fine granules (172.21 mg)]

| | | |
|---|---|---|
| controlled release fine granules | 110.34 | mg |
| (Production Example 21) | | |
| methacrylic acid/methyl acrylate/methyl | 52.88 | mg |
| methacrylate copolymer | | |
| polysorbate 80 | 1.0576 | mg |
| glycerol monostearate | 2.644 | mg |
| triethyl citrate | 5.288 | mg |
| total | 172.21 | mg |

### Production Example 23

### Production of Mannitol-Overcoated Controlled Release Fine Granules

Mannitol (16 g) was dissolved in purified water (90.67 g) to give a coating solution. The controlled release fine granules (344.43 g) obtained in Production Example 22 were coated with the aforementioned coating solution (106.67 g) by using a tumbling fluidized bed coater (SPIR-A-FLOW, manufactured by Freund Corporation). The coating conditions for mannitol overcoating were: inlet air temperature about 45° C., spray air pressure about 0.2 MPa, spray air volume about 30 Nl/min, BED pressure about 1.4 MPa, rotor rev rate about 300 rpm, spray rate about 3.0 g/min, spray position lower side. The obtained fine granules were then dried at 50° C. for 40 min in the tumbling fluidized bed coater and passed through a round sieve to give the outermost layer-coated fine granules with a particle size of 250 μm-425 μm.

[Composition of mannitol-overcoated controlled release fine
granules (180.21 mg)]

| | | |
|---|---|---|
| controlled release fine granules | 172.21 | mg |
| (Production Example 22) | | |
| mannitol | 8.0 | mg |
| total | 180.21 | mg |

### Production Example 24

### Production of Controlled Release Fine Granules

Purified water (625.625 g) was heated to 80° C., and polysorbate 80 (5.775 g), glycerol monostearate (14.4375 g) and triethyl citrate (14.4375 g) were dispersed therein. The suspension was cooled to room temperature, and then ethyl acrylate/methyl methacrylate copolymer dispersion (Eudragit NE30D) (481.25 g) and citric acid (1.4438 g) were added and the mixture was uniformly mixed. Furthermore, methacrylic acid/methyl acrylate/methyl methacrylate copolymer dispersion (Eudragit FS30D) (481.25 g) was added and the mixture was uniformly mixed to give a coating solution. The fine granules coated with intermediate layer (577.5 g) obtained in Production Example 2 were coated with the aforementioned coating solution (1624.2 g) by using a tumbling fluidized bed coater (MP-01, manufactured by POWREX CORPORATION). The coating conditions were: inlet air temperature 30° C., spray air pressure about 0.2 MPa, spray air volume about 90 Nl/min, inlet air volume about 0.5 m³/min, rotor rev rate about 500 rpm, to spray rate about 4 g/min, spray position lower side.

[Composition of controlled release fine granules (128.91 mg)]

| | | |
|---|---|---|
| fine granules coated with intermediate layer | 82.5 | mg |
| (Production Example 2) | | |
| methacrylic acid/methyl acrylate/methyl | 20.625 | mg |
| methacrylate copolymer | | |
| ethyl acrylate/methyl methacrylate copolymer | 20.625 | mg |
| polysorbate 80 | 0.825 | mg |
| glycerol monostearate | 2.0625 | mg |
| triethyl citrate | 2.0625 | mg |
| citric acid | 0.20625 | mg |
| total | 128.91 | mg |

### Production Example 25

### Production of Controlled Release Fine Granules

Purified water (274.42 g) was heated to 80° C., and polysorbate 80 (2.3388 g), glycerol monostearate (5.8470 g) and triethyl citrate (11.694 g) were dispersed therein. The suspension was cooled to room temperature, and added to methacrylic acid/methyl acrylate/methyl methacrylate copolymer dispersion (Eudragit FS30D) (389.8 g) and uniformly mixed to give a coating solution. The controlled release fine granules (257.81 g) obtained in Production Example 24 were coated with the aforementioned coating solution (684.1 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 300 rpm, spray rate about 3.0 g/min, spray position lower side.

[Composition of controlled release fine granules (197.32 mg)]

| | | |
|---|---|---|
| controlled release fine granules | 128.91 | mg |
| (Production Example 24) | | |
| methacrylic acid/methyl acrylate/methyl | 58.47 | mg |
| methacrylate copolymer | | |
| polysorbate 80 | 1.1694 | mg |
| glycerol monostearate | 2.9235 | mg |
| triethyl citrate | 5.847 | mg |
| total | 197.32 | mg |

### Production Example 26

### Production of Mannitol-Overcoated Controlled Release Fine Granules

Mannitol (17 g) was dissolved in purified water (96.3 g) to give a coating solution. The controlled release fine granules (394.63 g) obtained in Production Example 25 were coated with the aforementioned coating solution (113.3 g) by using a tumbling fluidized bed coater (SPIR-A-FLOW, manufactured by Freund Corporation). The coating conditions for mannitol overcoating were: inlet air temperature about 45° C., spray air pressure about 0.2 MPa, spray air volume about 30 Nl/min, BED pressure about 1.4 MPa, rotor rev rate about 300 rpm, spray rate about 3.0 g/min, spray position lower side. The obtained fine granules were then dried at 50° C. for 40 min in the tumbling fluidized bed coater and passed through a round sieve to give the outermost layer-coated fine granules with a particle size of 250 μm-425 μm.

[Composition of mannitol-overcoated controlled release fine
granules (205.82 mg)]

| | | |
|---|---|---|
| controlled release fine granules | 197.32 | mg |
| (Production Example 25) | | |
| mannitol | 8.5 | mg |
| total | 205.82 | mg |

### Production Example 27

### Production of Controlled Release Fine Granules

Purified water (726.88 g) was heated to 80° C., and polysorbate 80 (6.6 g), glycerol monostearate (16.5 g) and triethyl citrate (19.8 g) were dispersed therein. The suspension was cooled to room temperature, and then ethyl acrylate/methyl methacrylate copolymer dispersion (Eudragit NE30D) (440 g) and citric acid (1.32 g) were added and the mixture was uniformly mixed. Furthermore, methacrylic acid/methyl acrylate/methyl methacrylate copolymer dispersion (Eudragit FS30) (660 g) was added and the mixture was uniformly mixed to give a coating solution. The fine granules coated with intermediate layer (480.92 g) obtained in Production Example 2 were coated with a predetermined amount (1395.34 g) of the aforementioned coating solution (1871.1 g) by using a tumbling fluidized bed coater (MP-01, manufactured by POWREX CORPORATION). The coating conditions were: inlet air temperature about 30° C., spray air pressure about 0.2 MPa, spray air volume about 90 Nl/min, inlet air volume about 0.5 m³/min, rotor rev rate about 500 rpm, spray rate about 4 g/min, spray position lower side.

[Composition of controlled release fine granules (119.92 mg)]

| | | |
|---|---|---|
| fine granules coated with intermediate layer | 82.5 | mg |
| (Production Example 2) | | |
| methacrylic acid/methyl acrylate/methyl | 19.8 | mg |
| methacrylate copolymer | | |
| ethyl acrylate/methyl methacrylate copolymer | 13.2 | mg |
| polysorbate 80 | 0.66 | mg |
| glycerol monostearate | 1.65 | mg |
| triethyl citrate | 1.98 | mg |
| citric acid | 0.132 | mg |
| total | 119.92 | mg |

### Production Example 28

### Production of Controlled Release Fine Granules

Purified water (176.85 g) was heated to 80° C., and polysorbate 80 (1.5072 g), glycerol monostearate (3.768 g) and triethyl citrate (7.5361 g) were dispersed therein. The suspension was cooled to room temperature, and added to methacrylic acid/methyl acrylate/methyl methacrylate copolymer dispersion (Eudragit FS30D) (251.2 g) and uniformly mixed to give a coating solution. The controlled release fine granules (160.1 g) obtained in Production Example 27 were coated with the aforementioned coating solution (440.86 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 300 rpm, spray rate about 2.0 g/min, spray position lower side.

[Composition of controlled release fine granules (185.97 mg)]

| | | |
|---|---|---|
| controlled release fine granules | 119.92 | mg |
| (Production Example 27) | | |
| methacrylic acid/methyl acrylate/methyl | 56.45 | mg |
| methacrylate copolymer | | |
| polysorbate 80 | 1.129 | mg |
| glycerol monostearate | 2.8225 | mg |
| triethyl citrate | 5.645 | mg |
| total | 185.97 | mg |

### Production Example 29

### Production of Mannitol-Overcoated Controlled Release Fine Granules

Mannitol (11.3 g) was dissolved in purified water (64.3 g) to give a coating solution. The controlled release fine granules (248.27 g), which were obtained in Production Example 528 were coated with the aforementioned coating solution (75.6 g) by using a tumbling fluidized bed coater (SPIR-A-FLOW, manufactured by Freund Corporation). The coating conditions for mannitol overcoating were: inlet air temperature about 45° C., spray air pressure about 0.2 MPa, spray air volume about 30 Nl/min, BED pressure about 1.4 MPa, rotor rev rate about 300 rpm, spray rate about 3.0 g/min, spray position lower side. The obtained fine granules were then dried at 50° C. for 40 min in the tumbling fluidized bed coater and passed through a round sieve to give the outermost layer-coated fine granules with a particle size of 250 μm-425 μm.

[Composition of mannitol-overcoated controlled release fine
granules (194.47 mg)]

| | | |
|---|---|---|
| controlled release fine granules | 185.97 | mg |
| (Production Example 28) | | |
| mannitol | 8.5 | mg |
| total | 194.47 | mg |

### Production Example 30

### Production of Controlled Release Fine Granules

Purified water (831.11 g) was heated to 80° C., and polysorbate 80 (7.425 g), glycerol monostearate (18.5625 g) and triethyl citrate (25.9875 g) were dispersed therein. The suspension was cooled to room temperature, and then ethyl acrylate/methyl methacrylate copolymer dispersion (Eudragit NE30D) (371.25 g) and citric acid (1.1138 g) were added and the mixture was uniformly mixed. Furthermore, methacrylic acid/methyl acrylate/methyl methacrylate copolymer dispersion (Eudragit FS30D) (866.25 g) was added and the mixture was uniformly mixed to give a coating solution. The fine granules coated with intermediate layer (742.5 g) obtained in Production Example 2 were coated with the aforementioned coating solution (2121.69 g) by using a tumbling fluidized bed coater (MP-01, manufactured by POWREX CORPORATION). The coating conditions were: inlet air temperature 30° C., spray air pressure about 0.2 MPa, spray air volume about 90 Nl/min, inlet air volume about 0.5 m³/min, rotor rev rate about 500 rpm, spray rate about 4 g/min, spray position lower side.

[Composition of controlled release fine granules (129.65 mg)]

| | | |
|---|---|---|
| fine granules coated with intermediate layer | 82.5 | mg |
| (Production Example 2) | | |
| methacrylic acid/methyl acrylate/methyl | 28.875 | mg |
| methacrylate copolymer | | |
| ethyl acrylate/methyl methacrylate copolymer | 12.375 | mg |
| polysorbate 80 | 0.825 | mg |
| glycerol monostearate | 2.0625 | mg |
| triethyl citrate | 2.8875 | mg |
| citric acid | 0.12375 | mg |
| total | 129.65 | mg |

### Production Example 31

### Production of Controlled Release Fine Granules

Purified water (220.77 g) was heated to 80° C., and polysorbate 80 (1.8816 g), glycerol monostearate (4.704 g) and triethyl citrate (9.4080 g) were dispersed therein. The suspension was cooled to room temperature, and added to methacrylic acid/methyl acrylate/methyl methacrylate copolymer dispersion (Eudragit FS30D) (313.6 g) and uniformly mixed to give a coating solution. The controlled release fine granules (207.438 g) obtained in Production Example 30 were coated with the aforementioned coating solution (550.368 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 300 rpm, spray rate about 3.0 g/min, spray position lower side.

[Composition of controlled release fine granules (198.44 mg)]

| | | |
|---|---|---|
| controlled release fine granules | 129.65 | mg |
| (Production Example 30) | | |
| methacrylic acid/methyl acrylate/methyl | 58.8 | mg |
| methacrylate copolymer | | |
| polysorbate 80 | 1.176 | mg |
| glycerol monostearate | 2.94 | mg |
| triethyl citrate | 5.88 | mg |
| total | 198.44 | mg |

### Production Example 32

### Production of Mannitol-Overcoated Controlled Release Fine Granules

Mannitol (13.6 g) was dissolved in purified water (77.1 g) to give a coating solution. The controlled release fine granules (317.51 g) obtained in Production Example 31 were coated with the aforementioned coating solution (90.7 g) by using a tumbling fluidized bed coater (SPIR-A-FLOW, manufactured by Freund Corporation). The coating conditions for mannitol overcoating were: inlet air temperature about 45° C., spray air pressure about 0.2 MPa, spray air volume about 30 Nl/min, BED pressure about 1.4 MPa, rotor rev rate about 300 rpm, spray rate about 3.0 g/min, spray position lower side. The obtained fine granules were then dried at 50° C. for 40 min in the tumbling fluidized bed coater and passed through a round sieve to give the outermost layer-coated fine granules with a particle size of 250 μm-425 μm.

[Composition of mannitol-overcoated controlled release fine
granules (206.94 mg)]

| | | |
|---|---|---|
| controlled release fine granules | 198.44 | mg |
| (Production Example 31) | | |
| mannitol | 8.5 | mg |
| total | 206.94 | mg |

### Production Example 33

### Production of Controlled Release Fine Granules

Purified water (1108.7 g) was heated to 80° C., and polysorbate 80 (9.829 g), glycerol monostearate (24.57 g), triethyl citrate (34.4 g), yellow ferric oxide (0.8426 g) and ferric oxide (0.8426 g) were dispersed therein. The suspension was cooled to room temperature, and then ethyl acrylate/methyl methacrylate copolymer dispersion (Eudragit NE30D) (491.5 g) and citric acid (1.474 g) were added and the mixture was uniformly mixed. Furthermore, methacrylic acid/methyl acrylate/methyl methacrylate copolymer dispersion (Eudragit FS30D) (1147 g) was added and the mixture was uniformly mixed to give a coating solution. The fine granules coated with intermediate layer (900.1 g) obtained in Production Example 3 were coated with a predetermined amount (2466 g, 5% increased charge amount) of the aforementioned coating solution (2819 g) by using a tumbling fluidized bed coater (MP-10 TOKU-2 type, manufactured by POWREX CORPORATION). The coating conditions were: inlet air temperature 42° C., spray air pressure about 0.45 MPa, spray air volume about 120 Nl/min, inlet air volume about 1.3 m³/min, rotor rev rate about 550 rpm, spray rate about 8 g/min, spray position lower side.

[Composition of controlled release fine granules (154.1 mg)]

| | | |
|---|---|---|
| fine granules coated with intermediate layer | 101.25 | mg |
| (Production Example 3) | | |
| methacrylic acid/methyl acrylate/methyl | 32.25 | mg |
| methacrylate copolymer | | |
| ethyl acrylate/methyl methacrylate copolymer | 13.82 | mg |
| polysorbate 80 | 0.92 | mg |
| glycerol monostearate | 2.3 | mg |
| triethyl citrate | 3.22 | mg |
| citric acid | 0.138 | mg |
| yellow ferric oxide | 0.079 | mg |
| ferric oxide | 0.079 | mg |
| total | 154.1 | mg |

### Production Example 34

### Production of Controlled Release Fine Granules

Purified water (1630 g) was heated to 80° C., and polysorbate 80 (13.81 g), glycerol monostearate (34.51 g), triethyl citrate (69.03 g), yellow ferric oxide (1.211 g) and ferric oxide (1.211 g) were dispersed therein. The suspension was cooled to room temperature, and added to methacrylic acid/methyl acrylate/methyl methacrylate copolymer dispersion (Eudragit FS30D) (2301 g) and uniformly mixed to give a coating solution. The controlled release fine granules (1369.6 g) obtained in Production Example 33 were coated with a predetermined amount (3544 g, 5% increased charge amount) of the aforementioned coating solution (4050 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.3 m³/min, rotor rev rate about 550 rpm, spray rate about 10 g/min, spray position lower side.

[Composition of controlled release fine granules (230 mg)]

| | | |
|---|---|---|
| controlled release fine granules | 154.1 | mg |
| (Production Example 33) | | |
| methacrylic acid/methyl acrylate/methyl | 64.71 | mg |
| methacrylate copolymer | | |
| polysorbate 80 | 1.294 | mg |
| glycerol monostearate | 3.235 | mg |
| triethyl citrate | 6.471 | mg |
| yellow ferric oxide | 0.1136 | mg |
| ferric oxide | 0.1136 | mg |
| total | 230 | mg |

### Production Example 35

### Production of Mannitol-Overcoated Controlled Release Fine Granules

Mannitol (133.4 g) was dissolved in purified water (800.1 g) to give a coating solution. The controlled release fine granules (2044.7 g) obtained in Production Example 34 were coated with a predetermined amount (560 g, 5% increased charge amount) of the aforementioned coating solution (933.5 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.3 m³/min, rotor rev rate about 550 rpm, spray rate about 11 g/min, spray position lower side. The obtained fine granules were then dried and passed through a round sieve at 85° C. for about 40 min in the tumbling fluidized bed coater to give the outermost layer-coated fine granules with a particle size of 250 μm-425 μm.

[Composition of mannitol-overcoated controlled release
fine granules (239 mg)]

| | | |
|---|---|---|
| controlled release fine granules | 230 | mg |
| (Production Example 34) | | |
| mannitol | 9 | mg |
| total | 239 | mg |

### Reference Example 4

### Production of Controlled Release Fine Granules

Purified water (379.24 g) was heated to 80° C., and polysorbate 80 (1.0395 g) and glycerol monostearate (2.5988 g) were dispersed therein. The suspension was cooled to room temperature, and then ethyl acrylate/methyl methacrylate copolymer dispersion (Eudragit NE30D) (173.25 g) was added and the mixture was uniformly mixed to give a coating solution. The fine granules coated with intermediate layer (742.5 g) obtained in Production Example 2 were coated with the aforementioned coating solution (556.13 g) by using a tumbling fluidized bed coater (MP-01, manufactured by POWREX CORPORATION). The coating conditions were: inlet air temperature 30° C., spray air pressure about 0.2 MPa, spray air volume about 90 Nl/min, inlet air volume about 0.5 m³/min, rotor rev rate about 500 rpm, spray rate about 4 g/min, spray position lower side.

[Composition of controlled release fine granules (88.67925 mg)]

| | | |
|---|---|---|
| fine granules coated with intermediate layer | 82.5 | mg |
| (Production Example 2) | | |
| ethyl acrylate/methyl methacrylate copolymer | 5.775 | mg |
| polysorbate 80 | 0.1155 | mg |
| glycerol monostearate | 0.28875 | mg |
| total | 88.67925 | mg |

### Reference Example 5

### Production of Controlled Release Fine Granules

Purified water (259.4 g) was heated to 80° C., and polysorbate 80 (2.2109 g), glycerol monostearate (5.5272 g) and triethyl citrate (11.0544 g) were dispersed therein. The suspension was cooled to room temperature, and added to methacrylic acid/methyl acrylate/methyl methacrylate copolymer dispersion (Eudragit FS30D) (368.48 g) and uniformly mixed to give a coating solution. The controlled release fine granules (212.83 g) obtained in Reference Example 4 were coated with the aforementioned coating solution (646.68 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 500 rpm, spray rate about 3.0 g/min, spray position lower side.

[Composition of controlled release fine granules (142.57 mg)]

| | | |
|---|---|---|
| controlled release fine granules | 88.67925 | mg |
| (Reference Example 4) | | |
| methacrylic acid/methyl acrylate/methyl | 46.06 | mg |
| methacrylate copolymer | | |
| polysorbate 80 | 0.9212 | mg |
| glycerol monostearate | 2.303 | mg |
| triethyl citrate | 4.606 | mg |
| total | 142.57 | mg |

### Reference Example 6

### Production of Mannitol-Overcoated Controlled Release Fine Granules

Mannitol (16.8 g) was dissolved in purified water (95.2 g) to give a coating solution. The controlled release fine granules (342.17 g) obtained in Reference Example 5 were coated with the aforementioned coating solution (112 g) by using a tumbling fluidized bed coater (SPIR-A-FLOW, manufactured by Freund Corporation). The coating conditions for mannitol overcoating were: inlet air temperature about 50° C., spray air pressure about 0.2 MPa, spray air volume about 30 Nl/min, BED pressure about 1.4 MPa, rotor rev rate about 300 rpm, spray rate about 3.0 g/min, spray position lower side. The obtained fine granules were then dried at 50° C. for 40 min in the tumbling fluidized bed coater and passed through a round sieve to give the outermost layer-coated fine granules with a particle size of 250 μm-425 μm.

[Composition of mannitol-overcoated controlled
release fine granules (149.57 mg)]

| controlled release fine granules | | |
|---|---|---|
| (Reference Example 5) | 142.57 | mg |
| mannitol | 7.0 | mg |
| total | 149.57 | mg |

### Reference Example 7

### Production of Controlled Release Fine Granules

Purified water (715 g) was heated to 80° C., and polysorbate 80 (6.6 g), glycerol monostearate (16.5 g) and triethyl citrate (16.5 g) were dispersed therein. The suspension was cooled to room temperature, and then ethyl acrylate/methyl methacrylate copolymer dispersion (Eudragit NE30D) (550 g) and citric acid (1.65 g) were added and the mixture was uniformly mixed. Furthermore, methacrylic acid/ethyl acrylate copolymer dispersion (Eudragit L30D-55) (550 g) was added and the mixture was uniformly mixed to give a coating solution. The fine granules coated with intermediate layer (660 g) obtained in Production Example 2 were coated with the aforementioned coating solution (1856.25 g) by using a tumbling fluidized bed coater (MP-01, manufactured by POWREX CORPORATION). The coating conditions were: inlet air temperature 30° C., spray air pressure about 0.2 MPa, spray air volume about 90 Nl/min, inlet air volume about 0.5 m³/min, rotor rev rate about 500 rpm, spray rate about 4 g/min, spray position lower side.

[Composition of controlled release fine granules (128.91 mg)]

| | | |
|---|---|---|
| fine granules coated with intermediate layer | 82.5 | mg |
| (Production Example 2) | | |
| methacrylic acid/ethyl acrylate copolymer | 20.625 | mg |
| ethyl acrylate/methyl methacrylate copolymer | 20.625 | mg |
| polysorbate 80 | 0.825 | mg |
| glycerol monostearate | 2.0625 | mg |
| triethyl citrate | 2.0625 | mg |
| citric acid | 0.20625 | mg |
| total | 128.91 | mg |

### Reference Example 8

### Production of Controlled Release Fine Granules

Purified water (219.5 g) was heated to 80° C., and polysorbate 80 (1.871 g), glycerol monostearate (4.6776 g) and triethyl citrate (9.3552 g) were dispersed therein. The suspension was cooled to room temperature, and added to methacrylic acid/methyl acrylate/methyl methacrylate copolymer dispersion (Eudragit FS30D) (311.84 g) and uniformly mixed to give a coating solution. The controlled release fine granules (206.25 g) obtained in Reference Example 7 were coated with the aforementioned coating solution (547.28 g) by using a tumbling fluidized bed coater (SPIR-A-FLOW, manufactured by Freund Corporation). The coating conditions were: inlet air temperature about 33° C., spray air pressure about 0.2 MPa, spray air volume about 30 Nl/min, BED pressure about 1.4 MPa, rotor rev rate about 500 rpm, spray rate about 2.0 g/min, spray position lower side.

[Composition of controlled release fine granules (197.32 mg)]

| | | |
|---|---|---|
| controlled release fine granules | 128.91 | mg |
| (Reference Example 7) | | |
| methacrylic acid/methyl acrylate/methyl | 58.47 | mg |
| methacrylate copolymer | | |
| polysorbate 80 | 1.1694 | mg |
| glycerol monostearate | 2.9235 | mg |
| triethyl citrate | 5.847 | mg |
| total | 197.32 | mg |

### Reference Example 9

### Production of Mannitol-Overcoated Controlled Release Fine Granules

Mannitol (13.6 g) was dissolved in purified water (77.1 g) to give a coating solution. The controlled release fine granules (315.71 g) obtained in Reference Example 8 were coated with the aforementioned coating solution (90.7 g) by using a tumbling fluidized bed coater (SPIR-A-FLOW, manufactured by Freund Corporation). The coating conditions for mannitol overcoating were: inlet air temperature about 50° C., spray air pressure about 0.2 MPa, spray air volume about 30 Nl/min, BED pressure about 1.4 MPa, rotor rev rate about 300 rpm, spray rate about 4.0 g/min, spray position lower side. The obtained fine granules were then dried at 50° C. for 40 min in the tumbling fluidized bed coater and passed through a round sieve to give the outermost layer-coated fine granules with a particle size of 250 μm-425 μm.

[Composition of mannitol-overcoated controlled release
fine granules (205.82 mg)]

| | | |
|---|---|---|
| controlled release fine granules | 197.32 | mg |
| (Reference Example 8) | | |
| mannitol | 8.5 | mg |
| total | 205.82 | mg |

### Reference Example 10

### Production of Granules Containing a Pharmaceutically Active Ingredient

Compound X (1327 g), magnesium carbonate (972.4 g), granulated sugar (4716 g) and low-substituted hydroxypropyl cellulose (L-HPC-32, 732 g) were thoroughly mixed to give a spray agent. Sucrose/starch spherical granules (Nonpareil 101, 2258 g) were supplied into a centrifugation rolling granulator (CF-6005, Freund Corporation) and coated with a predetermined amount (7169 g) of the above-mentioned spray agent (7747.4 g) while spraying a hydroxypropylcellulose (HPC-L, 26.18 g) solution (2 w/w %) to give granules containing a pharmaceutically active ingredient. The obtained granules containing a pharmaceutically active ingredient were dried in vacuo at 40° C. for 16 hr, and passed through a round sieve to give granules with a particle size of 710 μm-1400 μm.

The coating conditions were: spray air volume about 40 L/min, inlet air volume about 1.2 m³/min, spray rate about 60 g/min, rotor rev rate about 125 rpm.

[Composition of granules containing a pharmaceutically
active ingredient (57.78 mg)]

| | | |
|---|---|---|
| Sucrose/starch spherical granules (Nonpareil 101) | 13.8 | mg |
| compound X | 7.5 | mg |
| magnesium carbonate | 5.5 | mg |
| granulated sugar | 26.68 | mg |
| low-substituted hydroxypropyl cellulose | 4.14 | mg |
| hydroxypropyl cellulose | 0.16 | mg |
| total | 57.78 | mg |

### Reference Example 11

### Production of Granules Coated with Intermediate Layer

The granules containing a pharmaceutically active ingredient obtained in Reference Example 10 was coated with an intermediate layer coating solution by using a fluidized bed coater (FD-S2, manufactured by POWREX CORPORATION), and then dried to give fine granules with the following composition. The intermediate layer coating solution was produced by dissolving hypromellose (TC-5E, 1131 g) in purified water (20427 g), and dispersing titanium oxide (685.2 g) and talc (452.6 g) in the obtained solution. The granules containing a pharmaceutically active ingredient (15120 g) obtained in Reference Example 10 were coated with a predetermined amount (19840 g) of the intermediate layer coating solution (22695.8 g) by using a fluidized bed coater (FD-S2, manufactured by POWREX CORPORATION). The coating conditions were: inlet air temperature about 60° C., spray air pressure about 0.5 MPa, spray air volume about 250 Nl/min, inlet air volume about 7 m³/min, spray rate about 70 g/min. After the completion of coating, the obtained granules were passed through a round sieve to give fine granules coated with intermediate layer with a particle size of 710 μm-1400 μm. The obtained granules were dried in vacuo at 40° C. for 16 hr.

[Composition of fine granules coated
with intermediate layer (65 mg)]

| | | |
|---|---|---|
| granules containing a pharmaceutically | 57.78 | mg |
| active ingredient (Reference Example 10) | | |
| hypromellose | 3.6 | mg |
| talc | 1.44 | mg |
| titanium oxide | 2.18 | mg |
| total | 65 | mg |

### Reference Example 12

### Production of Controlled Release Granules

Polyethylene glycol 6000 (268.2 g) and polysorbate 80 (122.9 g) were dissolved in purified water (12693 g), and titanium oxide (268.2 g), talc (810.3 g) and methacrylic acid/ethyl acrylate copolymer dispersion (Eudragit L30D-55) (8997 g) were dispersed in the obtained solution, and uniformly mixed to give a coating solution. The granules coated with intermediate layer (15270 g) obtained in Reference Example 11 were coated with a predetermined amount (20440 g) of the aforementioned coating solution (23159.6 g) by using a fluidized bed coater (FD-S2, manufactured by POWREX CORPORATION). The coating conditions were: inlet air temperature 60° C., spray air pressure about 0.5 MPa, spray air volume about 250 Nl/min, inlet air volume about 7 m³/min, spray rate about 70 g/min. The obtained granules were passed through a round sieve to give controlled release granules with a particle size of 850 μm-1400 μm. The obtained granules were dried in vacuo at 40° C. for 16 hr.

[Composition of controlled release fine granules (79.92 mg)]

| | | |
|---|---|---|
| fine granules coated with intermediate layer | 65 | mg |
| (Reference Example 11) | | |
| methacrylic acid/ethyl acrylate copolymer | 9.66 | mg |
| polyethylene glycol 6000 | 0.96 | mg |
| polysorbate 80 | 0.44 | mg |
| titanium oxide | 0.96 | mg |
| talc | 2.9 | mg |
| total | 79.92 | mg |

### Reference Example 13

### Production of granules containing a pharmaceutically active Ingredient

Compound X (3652 g), magnesium carbonate (972 g), granulated sugar (2394 g) and low-substituted hydroxypropyl cellulose (L-HPC-32, 729 g) were thoroughly mixed to give a spray agent. Sucrose/starch spherical granules (Nonpareil 101, 2250 g) were supplied into a centrifugation rolling granulator (CF-600S, Freund Corporation) and coated with a predetermined amount (7173 g) of the above-mentioned spray agent (7747 g) while spraying a hydroxypropylcellulose (HPC-L, 27 g) solution (2 w/w %) to give granules containing a pharmaceutically active ingredient. The obtained granules containing a pharmaceutically active ingredient were dried in vacuo at 40° C. for 16 hr, and passed through a round sieve to give granules with a particle size of 710 μm-1400 μm.

The coating conditions were: spray air volume about 40 L/min, inlet air volume about 1.0 m³/min, spray rate about 60 g/min, rotor rev rate about 125 rpm.

[Composition of granules containing a pharmaceutically
active ingredient (189 mg)]

| | | |
|---|---|---|
| Sucrose/starch spherical granules (Nonpareil 101) | 45 | mg |
| compound X | 67.5 | mg |
| magnesium carbonate | 18 | mg |
| granulated sugar | 44.46 | mg |
| low-substituted hydroxypropyl cellulose | 13.5 | mg |
| hydroxypropyl cellulose | 0.54 | mg |
| total | 189 | mg |

### Reference Example 14

### Production of Granules Coated with Intermediate Layer

The granules containing a pharmaceutically active ingredient obtained in Reference Example 13 was coated with an intermediate layer coating solution by using a fluidized bed coater (FD-S2, manufactured by POWREX CORPORATION), and then dried to give fine granules with the following composition. The intermediate layer coating solution was produced by dissolving hypromellose (TC-5EW, 1135 g) in purified water (20420 g), and dispersing titanium oxide (679.7 g) and talc (455 g) in the obtained solution. The granules containing a pharmaceutically active ingredient (15120 g) obtained in Reference Example 13 were coated with a predetermined amount (19860 g) of the intermediate layer coating solution (22689.7 g) by using a fluidized bed coater (FD-S2, manufactured by POWREX CORPORATION). The coating conditions were: inlet air temperature about 60° C., spray air pressure about 0.5 MPa, spray air volume about 250 Nl/min, inlet air volume about 7 m³/min, spray rate about 70 g/min. After the completion of coating, the obtained granules were passed through a round sieve to give fine granules coated with intermediate layer with a particle size of 710 μm-1400 μm. The obtained granules were dried in vacuo at 40° C. for 16 hr.

[Composition of fine granules coated with intermediate layer (212.64 mg)]

| | | |
|---|---|---|
| granules containing a pharmaceutically | 189 | mg |
| active ingredient (Reference Example 13) | | |
| hypromellose | 11.82 | mg |
| talc | 4.74 | mg |
| titanium oxide | 7.08 | mg |
| total | 212.64 | mg |

### Reference Example 15

### Production of Controlled Release Granules

Methacrylic acid/methyl methacrylate copolymer (Eudragit S100, 4115 g), methacrylic acid/methyl methacrylate copolymer (Eudragit L100, 1373 g) and triethyl citrate (547 g) were dissolved in a mixed solution of purified water (7899 g) and 99% ethanol (71100 g), and talc (2743 g) was dispersed in the obtained solution and uniformly mixed to give a coating solution. The granules coated with intermediate layer (15310 g) obtained in Reference Example 14 were coated with a predetermined amount (77160 g) of the aforementioned coating solution (87777 g) by using a fluidized bed coater (FD-S2, manufactured by POWREX CORPORATION). The coating conditions were: inlet air temperature 55° C., spray air pressure about 0.5 MPa, spray air volume about 280 Nl/min, inlet air volume about 7 m³/min, spray rate about 140 g/min. The obtained granules were passed through a round sieve to give controlled release granules with a particle size of 1000 μm-1700 μm. The obtained granules were dried in vacuo at 40° C. for 16 hr.

[Composition of controlled release fine granules (314.7 mg)]

| | | |
|---|---|---|
| fine granules coated with intermediate layer | 212.64 | mg |
| (Reference Example 14) | | |
| Eudragit S100 | 47.85 | mg |
| Eudragit L100 | 15.96 | mg |
| triethyl citrate | 6.36 | mg |
| talc | 31.89 | mg |
| total | 314.7 | mg |

### Reference Example 16

The granules (79.92 mg) obtained in Reference Example 12 and the granules (104.9 mg) obtained in Reference Example 15 were mixed, and talc (0.09 mg) and aerosil (0.09 mg) were added. These were filled in a HPMC capsule No. 0 to give a capsule formulation.

### Reference Example 17

### Production of Fine Granules Containing a Pharmaceutically Active Ingredient

Hydroxypropyl cellulose (360 g) was dissolved in purified water (4680 g), and then mannitol (270 g), talc (270 g), low-substituted hydroxypropyl cellulose (L-HPC-32, 180 g) and magnesium carbonate (360 g) were dispersed in this solution. Compound X (540 g) was uniformly dispersed in the obtained dispersion to give a coating solution. Lactose/crystalline cellulose spheres (Nonpareil 105T, 900 g) were coated with a predetermined amount (5550 g) from the compound X-containing coating solution (6660 g) by using a tumbling fluidized bed coater (MP-10 TOKU-2 type, manufactured by POWREX Corporation). The coating conditions were: inlet air temperature about 85° C., spray air pressure about 0.25 MPa, spray air volume about 80 Nl/min, inlet air volume about 0.7 m³/min, rotor rev rate about 500 rpm, spray rate about 15 g/min, spray position lower side.

[Composition of fine granules containing a
pharmaceutically active ingredient (85 mg)]

| | | |
|---|---|---|
| lactose/crystalline cellulose spheres | 30 | mg |
| (Nonpareil 105T) | | |
| compound X | 15 | mg |
| mannitol | 7.5 | mg |
| talc | 7.5 | mg |
| magnesium carbonate | 10 | mg |
| low-substituted hydroxypropyl cellulose | 5 | mg |
| hydroxypropyl cellulose | 10 | mg |
| total | 85 | mg |

### Reference Example 18

### Production of Fine Granules Coated with Intermediate Layer

The fine granules containing a pharmaceutically active ingredient obtained in Reference Example 17 was coated with an intermediate layer coating solution by using a tumbling fluidized bed coater (MP-10 TOKU-2 type, manufactured by POWREX CORPORATION), and then dried to give fine granules with the following composition. The intermediate layer coating solution was produced by dissolving hypromellose (TC-5E, 504 g) and mannitol (504 g) in purified water (5400 g), and dispersing titanium oxide (216 g), talc (216 g) and low-substituted hydroxypropyl cellulose (L-HPC-32, 360 g) in the obtained solution. The fine granules containing a pharmaceutically active ingredient (2550 g) obtained in Reference Example 17 were coated with a predetermined amount (6000 g) of the intermediate layer coating solution (7200 g) by using a tumbling fluidized bed coater (MP-10 TOKU-2 type, manufactured by POWREX CORPORATION). The coating conditions were: inlet air temperature about 85° C., spray air pressure about 0.35 MPa, spray air volume about 100 Nl/min, inlet air volume about 1.5 m³/min, rotor rev rate about 550 rpm, spray rate about 18 g/min, spray position lower side. After the completion of coating, the obtained fine granules were then dried at 85° C. for about 40 min in the tumbling fluidized bed coater and passed through a round sieve to give fine granules coated with intermediate layer with a particle size of 150 μm-350 μm.

[Composition of fine granules coated with intermediate
layer (110 mg)]

| | |
|---|---|
| fine granules containing a pharmaceutically | 85 mg |
| active ingredient (Reference Example 17) | |
| hypromellose | 14 mg |
| low-substituted hydroxypropyl cellulose | 10 mg |
| talc | 6 mg |
| titanium oxide | 6 mg |
| mannitol | 14 mg |
| total | 135 mg |

### Reference Example 19

### Production of Controlled Release Fine Granules

Purified water (1715.5 g) was heated to 80° C., and polysorbate 80 (14.26 g), glycerol monostearate (36.29 g), triethyl citrate (72.58 g), yellow ferric oxide (2.16 g) and ferric oxide (2.16 g) were dispersed therein. The suspension was cooled to room temperature, and added to methacrylic acid/methyl acrylate/methyl methacrylate copolymer dispersion (Eudragit FS30D) (2059 g) and methacrylic acid/ethyl acrylate copolymer dispersion (Eudragit L30D-55) (360 g), and uniformly mixed to give a coating solution. The fine granules coated with intermediate layer (1215 g) obtained in Reference Example 18 were coated with a predetermined amount (3733 g, 5% increased charge amount) of the aforementioned coating solution (4262 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 9 g/min, spray position lower side.

[Composition of controlled release fine granules (107 mg)]

| | | |
|---|---|---|
| fine granules coated with intermediate layer | 67.5 | mg |
| (Reference Example 18) | | |
| methacrylic acid/methyl acrylate/methyl | 28.6 | mg |
| methacrylate copolymer | | |
| methacrylic acid/ethyl acrylate copolymer | 5 | mg |
| polysorbate 80 | 0.7 | mg |
| glycerol monostearate | 1.7 | mg |
| triethyl citrate | 3.4 | mg |
| yellow ferric oxide | 0.05 | mg |
| ferric oxide | 0.05 | mg |
| total | 107 | mg |

### Reference Example 20

### Production of Mannitol-Overcoated Controlled Release Fine Granules

Mannitol (150 g) was dissolved in purified water (900 g) to give a coating solution. The controlled release fine granules (1961.6 g) obtained in Reference Example 19 were coated with a predetermined amount (630 g, 5% increased charge amount) of the aforementioned coating solution (1050 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.2 m³/min, rotor rev rate about 550 rpm, spray rate about 10 g/min, spray position lower side. The obtained fine granules were then dried at 85° C. for 40 min in the tumbling fluidized bed coater and passed through a round sieve to give the outermost layer-coated fine granules with a particle size of 250 μm-425 μm.

[Composition of mannitol-overcoated
controlled release fine granules (112 mg)]

| | | |
|---|---|---|
| controlled release fine granules | 107 | mg |
| (Reference Example 19) | 5 | mg |
| mannitol | 112 | mg |
| total | | |

### Reference Example 21

### Production of Controlled Release Fine Granules

Purified water (1715.5 g) was heated to 80° C., and polysorbate 80 (14.4 g), glycerol monostearate (36 g), triethyl citrate (72 g), yellow ferric oxide (2.16 g) and ferric oxide (2.16 g) were dispersed therein. The suspension was cooled to room temperature, and methacrylic acid/methyl acrylate/methyl methacrylate copolymer dispersion (Eudragit FS30D) (2302 g) and methacrylic acid/ethyl acrylate copolymer dispersion (Eudragit L30D-55) (120 g) were added and the mixture was uniformly mixed to give a coating solution. The fine granules coated with intermediate layer (1215 g) obtained in Production Example 3 were coated with a predetermined amount (3733 g, 5% increased charge amount) of the aforementioned coating solution (4264 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 9 g/min, spray position lower side.

[Composition of controlled release fine granules (160.5 mg)]

| | | |
|---|---|---|
| fine granules coated with intermediate layer | 101.25 | mg |
| (Production Example 3) | | |
| methacrylic acid/methyl acrylate/methyl | 48 | mg |
| methacrylate copolymer | | |
| methacrylic acid/ethyl acrylate copolymer | 2.5 | mg |
| polysorbate 80 | 1 | mg |
| glycerol monostearate | 2.5 | mg |
| triethyl citrate | 5.1 | mg |
| yellow ferric oxide | 0.075 | mg |
| ferric oxide | 0.075 | mg |
| total | 160.5 | mg |

### Reference Example 22

### Production of Mannitol-Overcoated Controlled Release Fine Granules

Mannitol (150 g) was dissolved in purified water (900 g) to give a coating solution. The controlled release fine granules (1961.6 g) obtained in Reference Example 21 were coated with a predetermined amount (630 g, 5% increased charge amount) of the aforementioned coating solution (1050 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.2 m³/min, rotor rev rate about 550 rpm, spray rate about 10 g/min, spray position lower side. The obtained fine granules were then dried at 85° C. for 40 min in the tumbling fluidized bed coater to give the outermost layer-coated fine granules with a particle size of 250 μm-425 μm.

[Composition of mannitol-overcoated
controlled release fine granules (168 mg)]

| | | |
|---|---|---|
| controlled release fine granules | 160.5 | mg |
| (Reference Example 21) | | |
| mannitol | 7.5 | mg |
| total | 168 | mg |

### Reference Example 23

### Production of Controlled Release Fine Granules

Purified water (1715.5 g) was heated to 80° C., and polysorbate 80 (14.4 g), glycerol monostearate (36 g), triethyl citrate (72 g), yellow ferric oxide (2.16 g) and ferric oxide (2.16 g) were dispersed therein. The suspension was cooled to room temperature, and methacrylic acid/methyl acrylate/methyl methacrylate copolymer dispersion (Eudragit FS30D) (2422 g) was added and the mixture was uniformly mixed to give a coating solution. The fine granules coated with intermediate layer (1215 g) obtained in Production Example 3 were coated with a predetermined amount (3733 g, 5% increased charge amount) of the aforementioned coating solution (4264 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 9 g/min, spray position lower side.

[Composition of controlled release fine granules (160.5 mg)]

| | | |
|---|---|---|
| fine granules coated with intermediate layer | 101.25 | mg |
| (Production Example 3) | | |
| methacrylic acid/methyl acrylate/methyl | 50.5 | mg |
| methacrylate copolymer | | |
| polysorbate 80 | 1 | mg |
| glycerol monostearate | 2.5 | mg |
| triethyl citrate | 5.1 | mg |
| yellow ferric oxide | 0.075 | mg |
| ferric oxide | 0.075 | mg |
| total | 160.5 | mg |

### Reference Example 24

### Production of Mannitol-Overcoated Controlled Release Fine Granules

Mannitol (150 g) was dissolved in purified water (900 g) to give a coating solution. The controlled release fine granules (1961.6 g) obtained in Reference Example 23 were coated with a predetermined amount (630 g, 5% increased charge amount) of the aforementioned coating solution (1050 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.2 m³/min, rotor rev rate about 550 rpm, spray rate about 10 g/min, spray position lower side. The obtained fine granules were then dried at 85° C. for 40 min in the tumbling fluidized bed coater and passed through a round sieve to give the outermost layer-coated fine granules with a particle size of 250 μm-425 μm.

[Composition of mannitol-overcoated
controlled release fine granules (168 mg)]

| | | |
|---|---|---|
| controlled release fine granules | 160.5 | mg |
| (Reference Example 23) | | |
| mannitol | 7.5 | mg |
| total | 168 | mg |

### Experimental Example 1

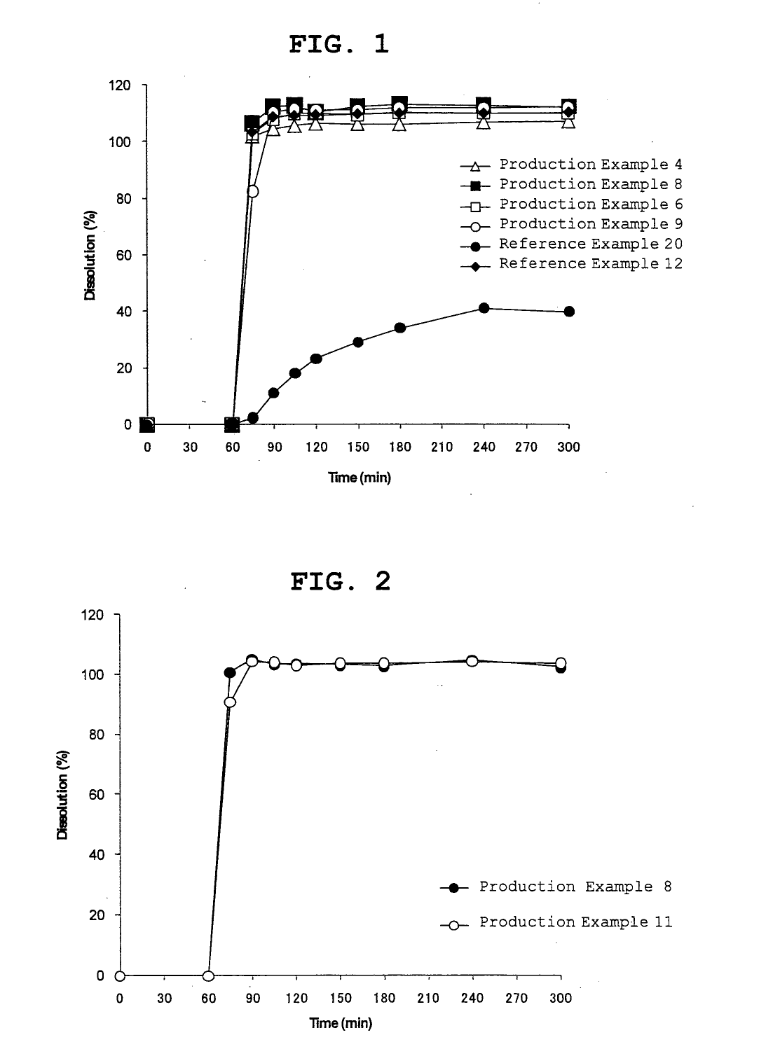

A dissolution test (test method (1)) was performed for the fine granules and granules obtained in Production Examples 4, 6, 8 and 9 and Reference Examples 12 and 20. The results are shown in FIG. 1.

### Experimental Example 2

A dissolution test (test method (1)) was performed for the fine granules obtained in Production Examples 8 and 11. The results are shown in FIG. 2.

### Experimental Example 3

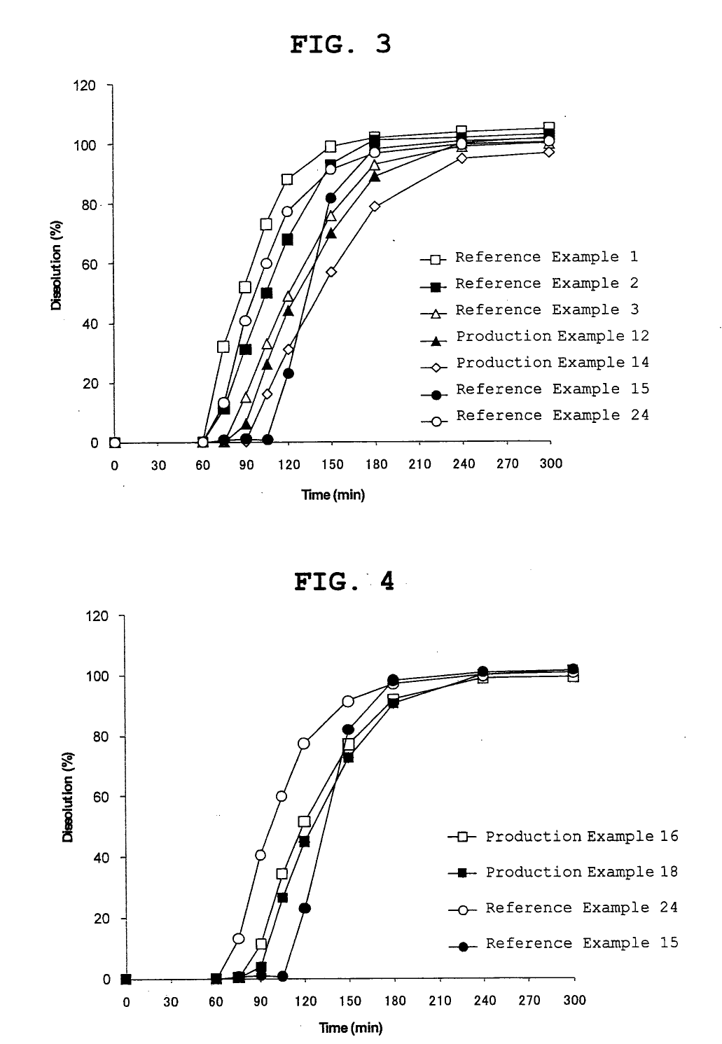

A dissolution test (test method (2)) was performed for the fine granules and granules obtained in Production Examples 12 and 14 and Reference Examples 1, 2, 3, 15 and 24. The results are shown in FIG. 3.

### Experimental Example 4

A dissolution test (test method (2)) was performed for the fine granules and granules obtained in Production Examples 16 and 18 and Reference Examples 15 and 24. The results are shown in FIG. 4.

### Experimental Example 5

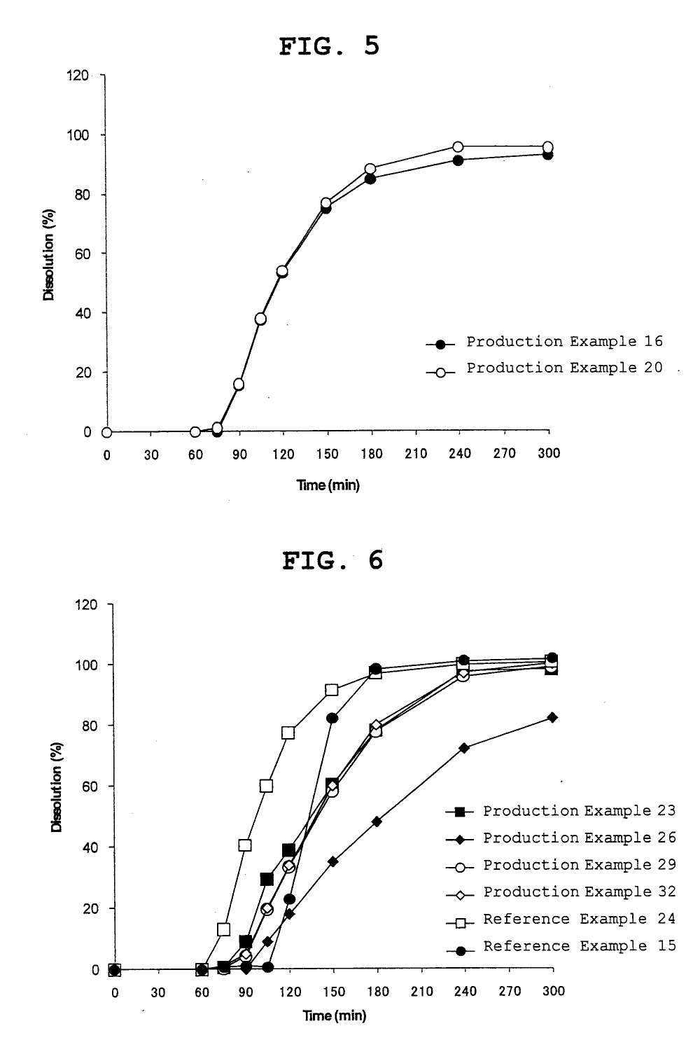

A dissolution test (test method (2)) was performed for the fine granules obtained in Production Examples 16 and 20. The results are shown in FIG. 5.

### Experimental Example 6

A dissolution test (test method (2)) was performed for the fine granules and granules obtained in Production Examples 23, 26, 29 and 32 and Reference Examples 15 and 24. The results are shown in FIG. 6.

### Experimental Example 7

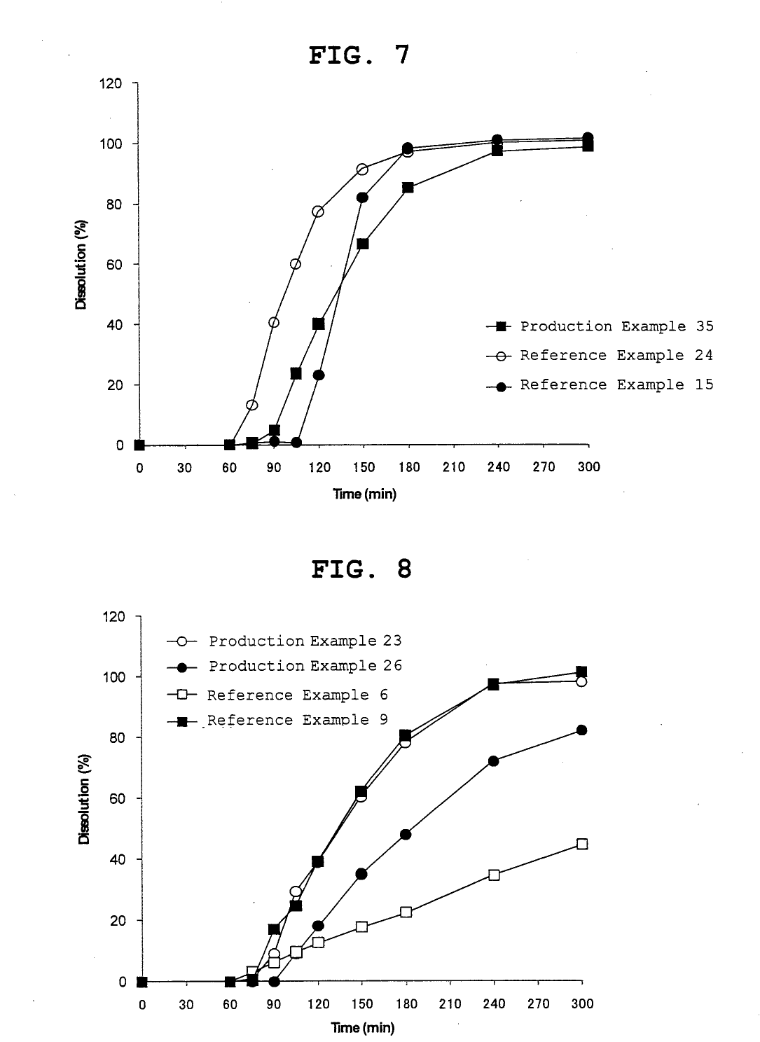

A dissolution test (test method (2)) was performed for the fine granules and granules obtained in Production Example 35 and Reference Examples 15 and 24. The results are shown in FIG. 7.

### Experimental Example 8

A dissolution test (test method (2)) was performed for the fine granules obtained in Production Examples 23 and 26 and Reference Examples 6 and 9. The results are shown in FIG. 8.

When the fine granules have a layer consisting only of an ethyl acrylate/methyl methacrylate copolymer, which is a diffusion-controlling polymer, as in Reference Example 6, the dissolution property is markedly dropped.

As in Production Example 23 and Production Example 26 that produce “fine granules (i)” contained in the tablet (II) of the present invention, when a methacrylic acid/methyl acrylate/methyl methacrylate copolymer is used as an enteric coating film in a mixed layer of an enteric coating film and a diffusion-controlling coating film, the drug release can be controlled with a smaller coating amount as compared to the use of a methacrylic acid/ethyl acrylate copolymer as an enteric coating film of a mixed layer of an enteric coating film and a diffusion-controlling coating film as in Reference Example 9. This is useful for preparing the fine granules having a size that prevents rough or dusty texture, and an orally disintegrating tablet containing fine granules, which is small and easy to swallow, can be produced.

### Experimental Example 9

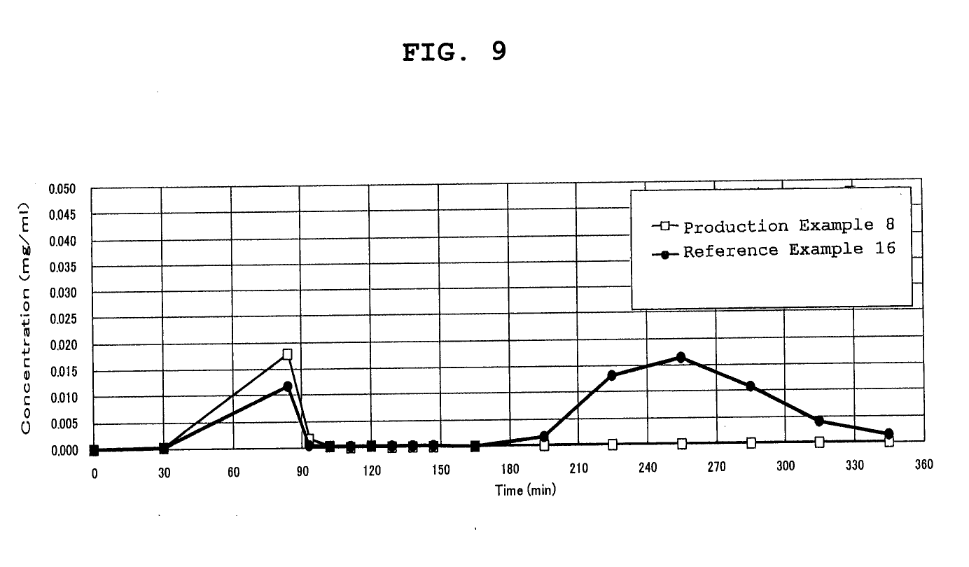

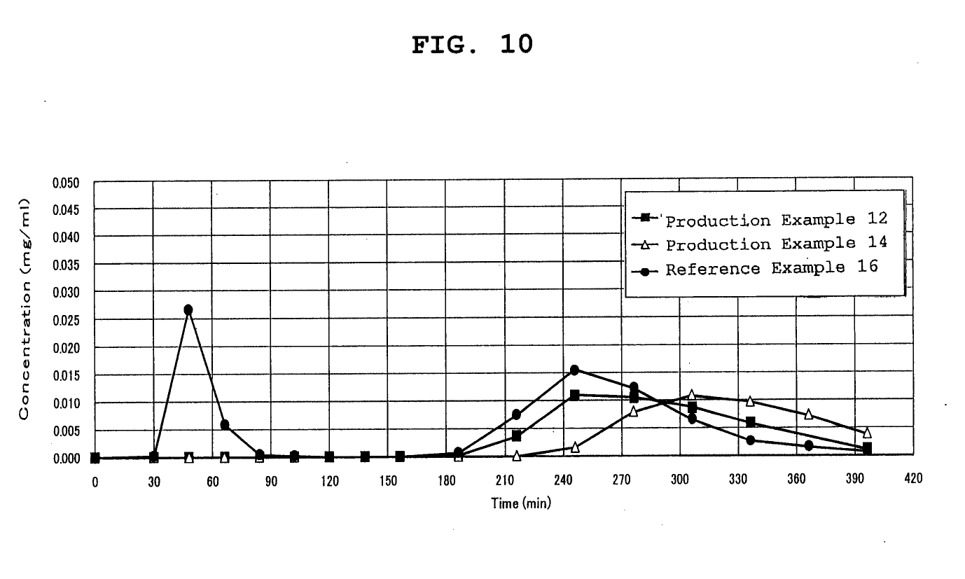

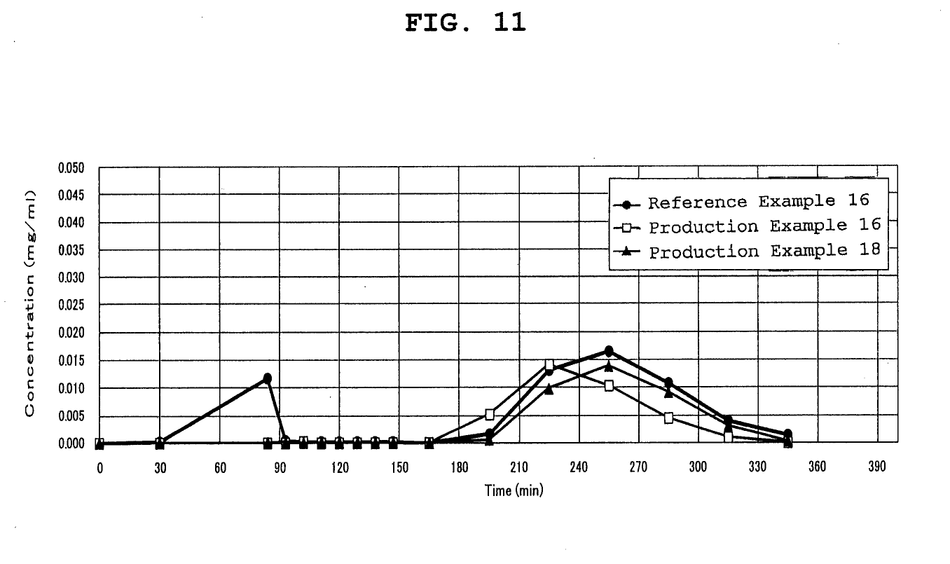

A dissolution test (test method (3)) was performed for the fine granules obtained in Production Example 8 and the capsule obtained in Reference Example 16. The results are shown in FIG. 9.

### Experimental Example 10

A dissolution test (test method (3)) was performed for the fine granules obtained in Production Examples 12 and 14 and the capsule obtained in Reference Example 16. The results are shown in FIG. 10.

### Experimental Example 11

A dissolution test (test method (3)) was performed for the fine granules obtained in Production Examples 16 and 18 and the capsule obtained in Reference Example 16. The results are shown in FIG. 11.

### Experimental Example 12

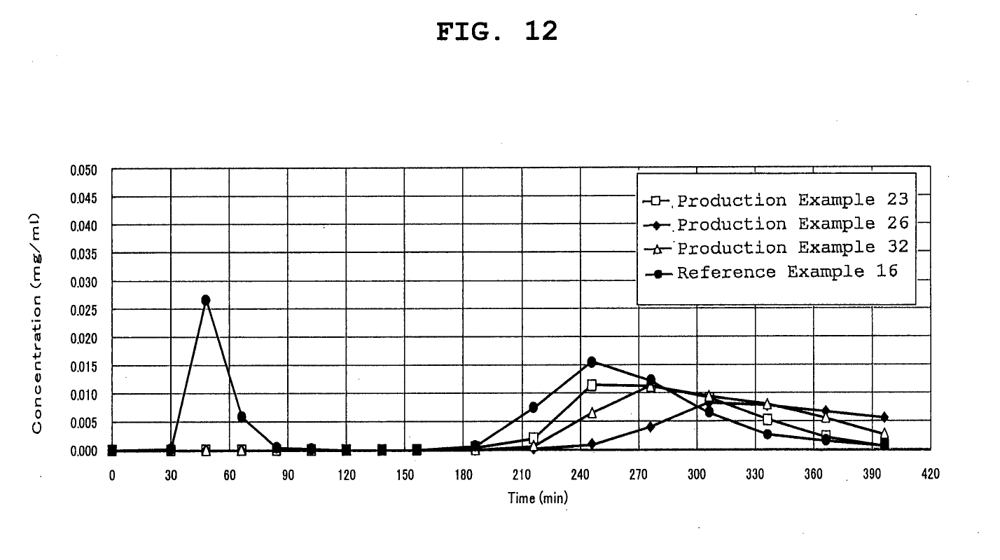

A dissolution test (test method (3)) was performed for the fine granules obtained in Production Examples 23, 26 and 32 and the capsule obtained in Reference Example 16. The results are shown in FIG. 12.

### Production Example 36

### Production of Outer Layer Component-Granulated Powder

Mannitol (2743 g), low-substituted hydroxypropyl cellulose (L-HPC-33, 432 g), crystalline cellulose (432 g) and crospovidone (216 g) were charged in a fluidized bed granulator (MP-10 TOKU-2 type, manufactured by POWREX CORPORATION), and they were granulated by spraying an aqueous solution of mannitol (216 g) and citric acid (43.2 g) in purified water (1440 g) and dried to give a granulated powder (4082 g).

[Composition of outer layer component-granulated
powder (314.07 mg]

| | |
|---|---|
| mannitol | 227.66 mg |
| low-substituted hydroxypropyl cellulose | 33.23 mg |
| crospovidone | 16.62 mg |
| crystalline cellulose | 33.23 mg |
| citric acid | 3.32 mg |
| total | 314.07 mg |

### Comparative Example 1

### Production of Orally Disintegrating Tablet

The mannitol-coated fine granules (1232 g) obtained in Reference Example 20, the mannitol-coated fine granules (1848 g) obtained in Reference Example 22, the outer layer component-granulated powder (3326 g) obtained in Production Example 36, sucralose (105.6 g), flavor (STRAWBERRY DURAROME) (35.2 g) and magnesium stearate (52.8 g) were mixed in a bag to give a mixed powder. The obtained mixed powder (6599.6 g) was tableted by using a rotary tableting machine (Correct 19K, Kikusui Seisakusho Ltd.) (600 mg/tablet, a 12 mmφ punch, flat-faced with beveled edge, tableting pressure 13 kN) to give the orally disintegrating tablet (600 mg) containing compound X (30 mg).

[Composition of orally disintegrating tablet (600 mg)]

| | | |
|---|---|---|
| mannitol-coated fine granules | 112 | mg |
| (Reference Example 20) | | |
| mannitol-coated fine granules | 168 | mg |
| (Reference Example 22) | | |
| outer layer component-granulated powder | 302.4 | mg |
| (Production Example 36) | | |
| sucralose | 9.6 | mg |
| flavor | 3.2 | mg |
| magnesium stearate | 4.8 | mg |
| total | 600 | mg |

The hardness, the disintegration time in the oral cavity and the disintegration time of the obtained tablet were 45.6 N, 35.3 seconds and 48 seconds, respectively. The dissolution rate of the obtained tablet in 0.1N HCl in 2 hours was 2%, showing superior acid resistance.

### Comparative Example 2

### Production of Orally Disintegrating Tablet

The mannitol-coated fine granules (1232 g) obtained in Reference Example 20, the mannitol-coated fine granules (1848 g) obtained in Reference Example 24, the outer layer component-granulated powder (3326 g) obtained in Production Example 36, sucralose (105.6 g), flavor (STRAWBERRY DURAROME) (35.2 g) and magnesium stearate (52.8 g) were mixed in a bag to give a mixed powder. The obtained mixed powder (6599.6 g) was tableted by using a rotary tableting machine (Correct 19K, Kikusui Seisakusho Ltd.) (600 mg/tablet, a 12 mmφ punch, flat-faced with beveled edge, tableting pressure 13 kN) to give the orally disintegrating tablet (600 mg) containing compound X (30 mg).

[Composition of orally disintegrating tablet (600 mg)]

| | | |
|---|---|---|
| mannitol-coated fine granules | 112 | mg |
| (Reference Example 20) | | |
| mannitol-coated fine granules | 168 | mg |
| (Reference Example 24) | | |
| outer layer component-granulated powder | 302.4 | mg |
| (Production Example 36) | | |
| sucralose | 9.6 | mg |
| flavor | 3.2 | mg |
| magnesium stearate | 4.8 | mg |
| total | 600 | mg |

The hardness, the disintegration time in the oral cavity and the disintegration time of the obtained tablet were 43.5 N, 35 seconds and 48 seconds, respectively. The dissolution rate of the obtained tablet in 0.1N HCl in 2 hours was 2%, showing superior acid resistance.

### Example 1

### Production of Orally Disintegrating Tablet

The mannitol-coated fine granules (385.2 g) obtained in Production Example 8, the mannitol-coated fine granules (1042.6 g) obtained in Production Example 16, the outer layer component-granulated powder (1507.5 g) obtained in Production Example 36, sucralose (48.38 g), flavor (STRAWBERRY DURAROME) (16.13 g) and magnesium stearate (24.19 g) were mixed in a bag to give a mixed powder. The obtained mixed powder (3024 g) was tableted by using a rotary tableting machine (Correct 19K, Kikusui Seisakusho Ltd.) (630 mg/tablet, a 13 mmφ punch, flat-faced with beveled edge, tableting pressure 19.5 kN) to give the orally disintegrating tablet (630 mg) containing compound X (30 mg) of the present invention.

[Composition of orally disintegrating tablet (630
mg)]

| | | |
|---|---|---|
| mannitol-coated fine granules | 80.25 | mg |
| (Production Example 8) | | |
| mannitol-coated fine granules | 217.2 | mg |
| (Production Example 16) | | |
| outer layer component-granulated powder | 314.07 | mg |
| (Production Example 36) | | |
| sucralose | 10.08 | mg |
| flavor | 3.36 | mg |
| magnesium stearate | 5.04 | mg |
| total | 630 | mg |

The hardness, the disintegration time in the oral cavity and the disintegration time of the obtained tablet were 47 N, 43.3 seconds and 44 seconds, respectively. The dissolution rate of the obtained tablet in 0.1N HCl in 1 hour was 0.8%, showing superior acid resistance.

### Example 2

### Production of Orally Disintegrating Tablet

The mannitol-coated fine granules (361.1 g) obtained in Production Example 8, the mannitol-coated fine granules (1086.8 g) obtained in Production Example 18, the outer layer component-granulated powder (1522.4 g) obtained in Production Example 36, sucralose (48.96 g), flavor (STRAWBERRY DURAROME) (16.34 g) and magnesium stearate (24.48 g) were mixed in a bag to give a mixed powder. The obtained mixed powder (3060 g) was tableted by using a rotary tableting machine (Correct 19K, Kikusui Seisakusho Ltd.) (680 mg/tablet, a 13 mmφ punch, flat-faced with beveled edge, tableting pressure 19.5 kN) to give the orally disintegrating tablet (680 mg) containing compound X (30 mg) of the present invention.

[Composition of orally disintegrating tablet (680 mg)]

| | | |
|---|---|---|
| mannitol-coated fine granules | 80.25 | mg |
| (Production Example 8) | | |
| mannitol-coated fine granules | 241.5 | mg |
| (Production Example 18) | | |
| outer layer component-granulated powder | 338.3 | mg |
| (Production Example 36) | | |
| sucralose | 10.88 | mg |
| flavor | 3.63 | mg |
| magnesium stearate | 5.44 | mg |
| total | 680 | mg |

The hardness, the disintegration time in the oral cavity and the disintegration time of the obtained tablet were 50 N, 46.7 seconds and 51.3 seconds, respectively. The dissolution rate of the obtained tablet in 0.1N HCl in 1 hour was 1.0%, showing superior acid resistance.

### Example 3

### Production of Orally Disintegrating Tablet

The mannitol-coated fine granules (361.1 g) obtained in Production Example 8, the mannitol-coated fine granules (1075.5 g) obtained in Production Example 35, the outer layer component-granulated powder (1490 g) obtained in Production Example 36, sucralose (48.24 g), flavor (STRAWBERRY DURAROME) (16.07 g) and magnesium stearate (24.12 g) were mixed in a bag to give a mixed powder. The obtained mixed powder (3015 g) was tableted by using a rotary tableting machine (Correct 19K, Kikusui Seisakusho Ltd.) (670 mg/tablet, a 13 mmφ punch, flat-faced with beveled edge, tableting pressure 19.0 kN) to give the orally disintegrating tablet (670 mg) containing compound X (30 mg) of the present invention.

[Composition of orally disintegrating tablet (670 mg)]

| | | |
|---|---|---|
| mannitol-coated fine granules | 80.25 | mg |
| (Production Example 8) | | |
| mannitol-coated fine granules | 239 | mg |
| (Production Example 35) | | |
| outer layer component-granulated powder | 331.1 | mg |
| (Production Example 36) | | |
| sucralose | 10.72 | mg |
| flavor | 3.57 | mg |
| magnesium stearate | 5.36 | mg |
| total | 670 | mg |

The hardness, the disintegration time in the oral cavity and the disintegration time of the obtained tablet were 43.9 N, 38.7 seconds and 37.5 seconds, respectively. The dissolution rate of the obtained tablet in 0.1N HCl in 1 hour was 1.1%, showing superior acid resistance.

### Example 4

### Production of Orally Disintegrating Tablet

The mannitol-coated fine granules (391.3 g) obtained in Production Example 11, the mannitol-coated fine granules (1053.7 g) obtained in Production Example 20, the outer layer component-granulated powder (1520.3 g) obtained in Production Example 36, sucralose (48.88 g), flavor (STRAWBERRY DURAROME) (16.31 g) and magnesium stearate (24.44 g) were mixed in a bag to give a mixed powder. The obtained mixed powder (3055 g) was tableted by using a rotary tableting machine (Correct 19K, Kikusui Seisakusho Ltd.) (650 mg/tablet, a 13 mmφ punch, flat-faced with beveled edge, tableting pressure 19.5 kN) to give the orally disintegrating tablet (650 mg) containing compound X (30 mg) of the present invention.

[Composition of orally disintegrating tablet
(650 mg)]

| | | |
|---|---|---|
| mannitol-coated fine granules | 83.26 | mg |
| (Production Example 11) | | |
| mannitol-coated fine granules | 224.2 | mg |
| (Production Example 20) | | |
| outer layer component-granulated powder | 323.47 | mg |
| (Production Example 36) | | |
| sucralose | 10.4 | mg |
| flavor | 3.47 | mg |
| magnesium stearate | 5.2 | mg |
| total | 650 | mg |

The hardness, the disintegration time in the oral cavity and the disintegration time of the obtained tablet were 45.9 N, 25.3 seconds and 22.6 seconds, respectively. The dissolution rate of the obtained tablet in 0.1N HCl in 1 hour was 1.2%, showing superior acid resistance.

### Experimental Example 13

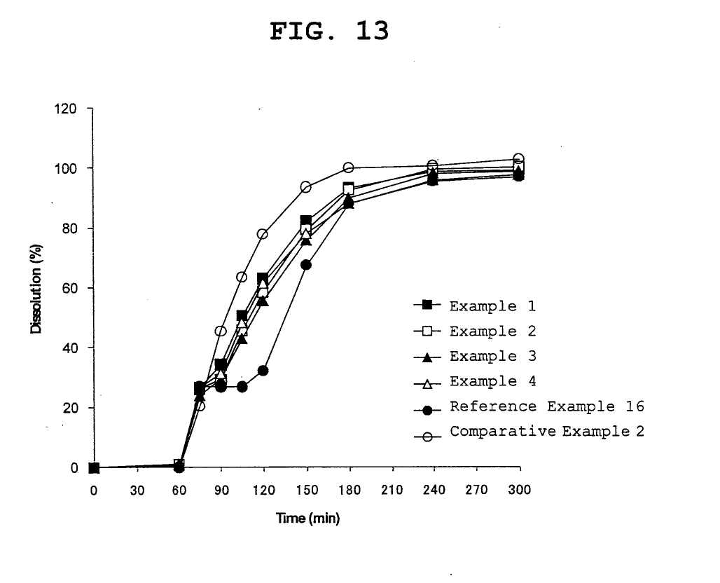

A dissolution test (test method (2)) was performed for the formulations obtained in Examples 1, 2, 3 and 4, Reference Example 16 and Comparative Example 2. The results are shown in FIG. 13.

### Experimental Example 14

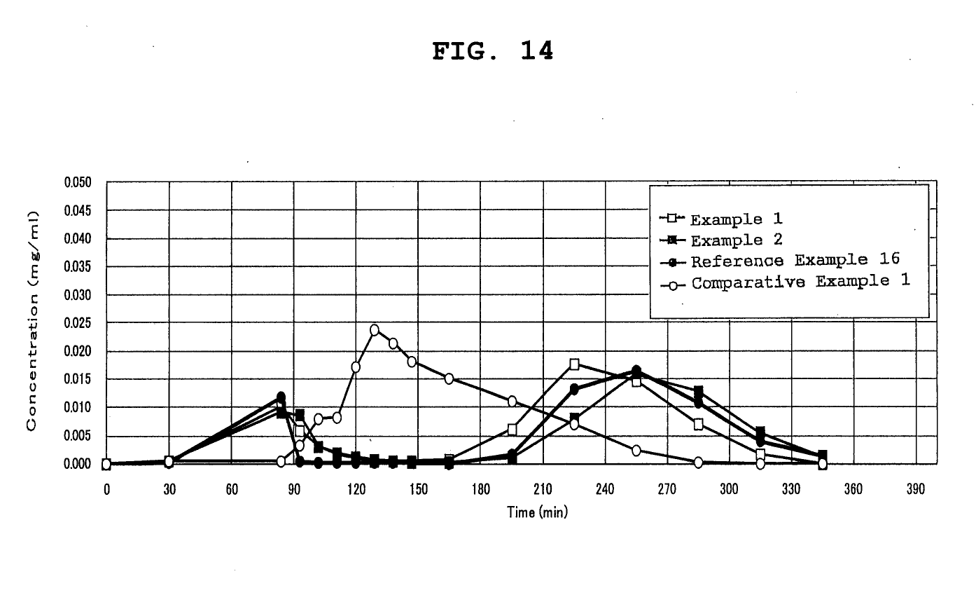

A dissolution test (test method (3)) was performed for the formulations obtained in Examples 1 and 2, Reference Example 16 and Comparative Example 1. The results are shown in FIG. 14.

### Experimental Example 15

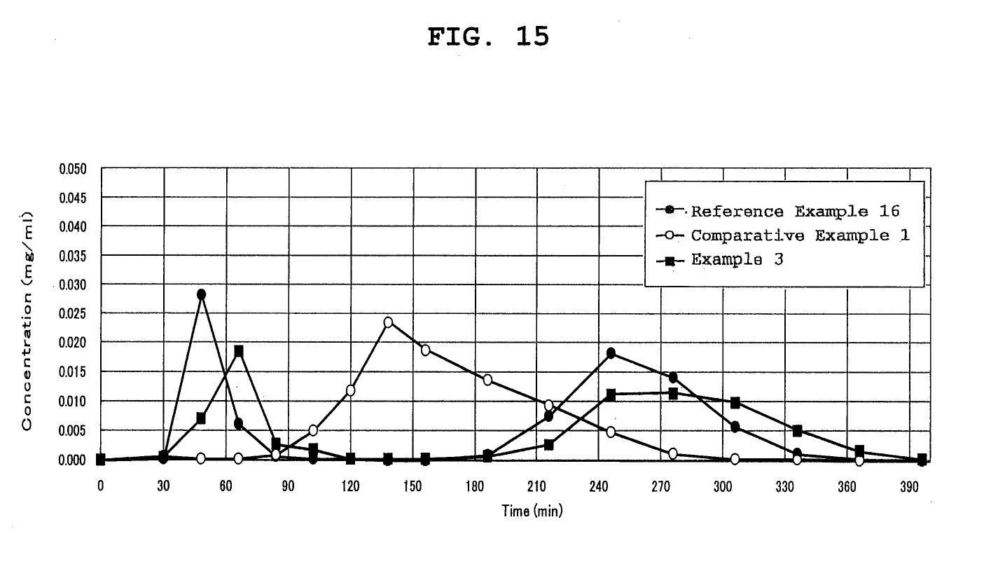

A dissolution test (test method (3)) was performed for the formulations obtained in Example 3, Reference Example 16 and Comparative Example 1. The results are shown in FIG. 15.

### Reference Example 25

### Production of Granules Containing a Pharmaceutically Active Ingredient

Compound X (3645 g), magnesium carbonate (972 g), granulated sugar (2401 g) and low-substituted hydroxypropyl cellulose (L-HPC-32, 729 g) were thoroughly mixed to give a spray agent. Sucrose/starch spherical granules (Nonpareil 101, 2250 g) were supplied into a centrifugation rolling granulator (CF-6005, Freund Corporation) and coated with a predetermined amount (7173 g) of the above-mentioned spray agent (7747 g) while spraying a hydroxypropylcellulose (HPC-L, 27 g) solution (2 w/w %) to give granules containing a pharmaceutically active ingredient. The obtained granules containing a pharmaceutically active ingredient were dried in vacuo at 40° C. for 16 hr, and passed through a round sieve to give granules with a particle size of 710 μm-1400 μm.

The coating conditions were: spray air volume about 40 L/min, inlet air volume about 1.0 m³/min, spray rate about 60 g/min, rotor rev rate about 125 rpm.

[Composition of granules containing a pharmaceutically
active ingredient (57.78 mg)]

| | | |
|---|---|---|
| Sucrose/starch spherical granules (Nonpareil 101) | 15.0 | mg |
| compound X | 22.5 | mg |
| magnesium carbonate | 6.0 | mg |
| granulated sugar | 14.82 | mg |
| low-substituted hydroxypropyl cellulose | 4.5 | mg |
| hydroxypropyl cellulose | 0.18 | mg |
| total | 63.0 | mg |

### Reference Example 26

### Production of Granules Coated with Intermediate Layer

The granules containing a pharmaceutically active ingredient obtained in Reference Example 25 was coated with an intermediate layer coating solution by using a fluidized bed coater (FD-S2, manufactured by POWREX CORPORATION), and then dried to give fine granules with the following composition. The intermediate layer coating solution was produced by dissolving hypromellose (TC-5EW, 1135 g) in purified water (20420 g), and dispersing titanium oxide (679.7 g) and talc (455.0 g) in the obtained solution. The granules containing a pharmaceutically active ingredient (15120 g) obtained in Reference Example 25 were coated with a predetermined amount (19860 g) of the intermediate layer coating solution (22689.7 g) by using a fluidized bed coater (FD-S2, manufactured by POWREX CORPORATION). The coating conditions were: inlet air temperature about 60° C., spray air pressure about 0.5 MPa, spray air volume about 250 Nl/min, inlet air volume about 7 m³/min, spray rate about 70 g/min. After the completion of coating, the obtained granules were passed through a round sieve to give fine granules coated with intermediate layer with a particle size of 710 μm-1400 μm. The obtained granules were dried in vacuo at 40° C. for 16 hr.

[Composition of fine granules coated with
intermediate layer (65 mg)]

| | |
|---|---|
| granules containing a pharmaceutically active | 63.00 mg |
| ingredient (Reference Example 25) | |
| hypromellose | 3.94 mg |
| talc | 1.58 mg |
| titanium oxide | 2.36 mg |
| total | 70.88 mg |

### Reference Example 27

### Production of Controlled Release Granules

Polyethylene glycol 6000 (273.0 g) and polysorbate 80 (124.8 g) were dissolved in purified water (12600 g), and titanium oxide (273.0 g), talc (759.2 g) and methacrylic acid/ethyl acrylate copolymer dispersion (Eudragit L30D-55) (9126 g) were dispersed in the obtained solution, and uniformly mixed to give a coating solution. The granules coated with intermediate layer (15310 g) obtained in Reference Example 26 were coated with a predetermined amount (20200 g) of the aforementioned coating solution (23156.0 g) by using a fluidized bed coater (FD-S2, manufactured by POWREX CORPORATION). The coating conditions were: inlet air temperature 60° C., spray air pressure about 0.5 MPa, spray air volume about 250 Nl/min, inlet air volume about 7 m³/min, spray rate about 70 g/min. The obtained granules were passed through a round sieve to give controlled release granules with a particle size of 850 μm-1400 μm. The obtained granules were dried in vacuo at 40° C. for 16 hr.

[Composition of controlled release fine granules (79.92 mg)]

| | |
|---|---|
| fine granules coated with intermediate layer | 70.88 mg |
| (Reference Example 26) | |
| methacrylic acid/ethyl acrylate copolymer | 10.53 mg |
| polyethylene glycol 6000 | 1.05 mg |
| polysorbate 80 | 0.48 mg |
| titanium oxide | 1.05 mg |
| talc | 2.92 mg |
| total | 86.91 mg |

### Comparative Example 3

### Production of Orally Disintegrating Tablet

The controlled release granules (2.098 g) obtained in Reference Example 15, the controlled release granules (0.5794 g) obtained in Reference Example 27, the outer layer component-granulated powder (2.977 g) obtained in Production Example 36 and magnesium stearate (0.0456 g) were mixed in a bag to give a mixed powder. The obtained mixed powder (5.7 g) was tableted by using an Autograph tableting machine (AG-IS, SHIMADZU Corporation) (285 mg/tablet, a 9 mmφ punch, flat-faced with beveled edge, tableting pressure 10 kN) to give the orally disintegrating tablet (285 mg) containing compound X (30 mg).

[Composition of orally disintegrating tablet (285 mg)]

| | |
|---|---|
| controlled release granules (Reference Example 15) | 104.9 mg |
| controlled release granules (Reference Example 27) | 28.97 mg |
| outer layer component-granulated powder | |
| (Production Example 36) | 148.85 mg |
| magnesium stearate | 2.28 mg |
| total | 285 mg |

The dissolution rate of the drug in the obtained tablet in 0.1N HCl in 1 hour was 17.4%. The controlled release films used in Reference Example 15 and Reference 27 could not ensure acid resistance after tableting, and application of the fine granules and granules having the film to an orally disintegrating tablet is difficult.

### Production Example 37

### Production of Fine Granules Containing a Pharmaceutically Active Ingredient

Hydroxypropyl cellulose (13.2 kg) was dissolved in purified water (184.8 kg), and low-substituted hydroxypropyl cellulose (L-HPC-32, 6.6 kg) and magnesium carbonate (13.2 kg) were dispersed in this solution. Compound X (39.6 kg) was uniformly dispersed in the obtained dispersion to give a coating solution. Lactose/crystalline cellulose spheres (Nonpareil 105T 39.6 kg) were coated with this compound X-containing coating solution (257.4 kg) by using a tumbling fluidized bed coater (MP-400, manufactured by POWREX Corporation). The coating conditions were: inlet air temperature about 70° C., spray air volume about 1200 Nl/min/gun, inlet air volume about 55.0 Nm³/min, rotor rev rate about 100 rpm, spray rate about 320 mL/min/gun, spray position lower side.

[Composition of fine granules containing a
pharmaceutically active ingredient (85 mg)]

| | |
|---|---|
| lactose/crystalline cellulose spheres | 30 mg |
| (Nonpareil 105T) | |
| compound X | 30 mg |
| magnesium carbonate | 10 mg |
| low-substituted hydroxypropyl cellulose | 5 mg |
| hydroxypropyl cellulose | 10 mg |
| total | 85 mg |

### Production Example 38

### Production of fine granules coated with intermediate layer

The fine granules containing a pharmaceutically active ingredient obtained in Production Example 37 was coated with an intermediate layer coating solution by using a tumbling fluidized bed coater (MP-400, manufactured by POWREX CORPORATION), and then dried to give fine granules with the following composition. The intermediate layer coating solution was produced by dissolving hypromellose (TC-5E, 18.48 kg) and mannitol (18.48 kg) in purified water (198 kg), and dispersing titanium oxide (7.92 kg), talc (7.92 kg) and low-substituted hydroxypropyl cellulose (L-HPC-32, 13.2 kg) in the obtained solution. The fine granules containing a pharmaceutically active ingredient (112.2 kg) obtained in Production Example 37 were coated with the intermediate layer coating solution (264 kg) by using a tumbling fluidized bed coater (MP-400, manufactured by POWREX CORPORATION). The coating conditions were: inlet air temperature about 75° C., spray air volume about 1100 Nl/min/gun, inlet air volume about 55 Nm³/min, rotor rev rate about 120 rpm, spray rate about 270 mL/min/gun, spray position lower side. After the completion of coating, the obtained fine granules were then dried at 85° C. for about 20 min in the tumbling fluidized bed coater and passed through a round sieve to give the intermediate layer-coated fine granules with a particle size of 150 μm-350 μm.

[Composition of fine granules coated with
intermediate layer (110 mg)]

| | |
|---|---|
| fine granules containing a pharmaceutically | 85 mg |
| active ingredient (Production Example 37) | |
| hypromellose | 14 mg |
| low-substituted hydroxypropyl cellulose | 10 mg |
| talc | 6 mg |
| titanium oxide | 6 mg |
| mannitol | 14 mg |
| total | 135 mg |

### Production Example 39

### Production of Controlled Release Fine Granules

Purified water (105.3 kg) was heated to 70° C., and polysorbate 80 (1.102 kg), glycerol monostearate (2.756 kg), yellow ferric oxide (0.08845 kg) and ferric oxide (0.08845 kg) were dispersed therein. The suspension was cooled to room temperature, and then ethyl acrylate/methyl methacrylate copolymer dispersion (Eudragit NE30D) (15.31 kg) and citric acid (0.046 kg) were added and the mixture was uniformly mixed. Furthermore, methacrylic acid/ethyl acrylate copolymer dispersion (Eudragit L30D-55) (137.8 kg), triethyl citrate (9.185 kg) and purified water (21.1 kg) were added and the mixture was uniformly mixed to give a coating solution. The fine granules coated with intermediate layer (43.74 kg) obtained in Production Example 38 were coated with the aforementioned coating solution (292.7 kg, 5% increased charge amount) by using a tumbling fluidized bed coater (MP-400, manufactured by POWREX CORPORATION). The coating conditions were: inlet air temperature about 80° C., spray air volume about 1200 Nl/min/gun, inlet air volume about 50 Nm³/min, rotor rev rate about 150 rpm, spray rate about 250 mL/min/gun, spray position lower side.

[Composition of controlled release fine granules (77.2488 mg)]

| | | |
|---|---|---|
| fine granules coated with intermediate layer | 33.75 | mg |
| (Production Example 38) | | |
| methacrylic acid/ethyl acrylate copolymer | 30.375 | mg |
| ethyl acrylate/methyl methacrylate copolymer | 3.375 | mg |
| polysorbate 80 | 0.81 | mg |
| glycerol monostearate | 2.025 | mg |
| triethyl citrate | 6.75 | mg |
| citric acid | 0.0338 | mg |
| yellow ferric oxide | 0.065 | mg |
| ferric oxide | 0.065 | mg |
| total | 77.2488 | mg |

### Production Example 40

### Production of Controlled Release Fine Granules

Purified water (52.7 kg) was heated to 70° C., and polysorbate 80 (0.5552 kg), glycerol monostearate (1.388 kg), yellow ferric oxide (0.04442 kg) and ferric oxide (0.04442 kg) were dispersed therein. The suspension was cooled to room temperature, and then ethyl acrylate/methyl methacrylate copolymer dispersion (Eudragit NE30D) (7.711 kg) and citric acid (0.02313 kg) were added and the mixture was uniformly mixed. Furthermore, methacrylic acid/ethyl acrylate copolymer dispersion (Eudragit L30D-55) (69.4 kg), polyethylene glycol (2.313 kg) and purified water (4.2 kg) were added and the mixture was uniformly mixed to give a coating solution. The controlled release fine granules (100.1 kg) obtained in Production Example 39 were coated with a predetermined amount (20.4 kg, 5% increased charge amount) of the aforementioned coating solution (138.5 kg) by using a tumbling fluidized bed coater (MP-400, manufactured by POWREX CORPORATION). The coating conditions were: Inlet air temperature about 80° C., spray air volume about 1200 Nl/min/gun, inlet air volume about 50 Nm³/min, rotor rev rate about 150 rpm, spray rate about 250 mL/min/gun, spray position lower side.

[Composition of controlled release fine granules (80.2209 mg)]

| | | |
|---|---|---|
| controlled release fine granules | 77.2488 | mg |
| (Production Example 39) | | |
| methacrylic acid/ethyl acrylate copolymer | 2.25 | mg |
| ethyl acrylate/methyl methacrylate copolymer | 0.25 | mg |
| polysorbate 80 | 0.06 | mg |
| glycerol monostearate | 0.15 | mg |
| polyethylene glycol | 0.25 | mg |
| citric acid | 0.0025 | mg |
| yellow ferric oxide | 0.0048 | mg |
| ferric oxide | 0.0048 | mg |
| total | 80.2209 | mg |

### Production Example 41

### Production of Mannitol-Overcoated Controlled Release Fine Granules

Mannitol (4.054 kg) was dissolved in purified water (24.3 kg) to give a coating solution. The controlled release fine granules (104.0 kg) obtained in Production Example 40 were coated with the aforementioned coating solution (28.354 kg, 5% increased charge amount) by using a tumbling fluidized bed coater (MP-400, manufactured by POWREX Corporation). The coating conditions for mannitol overcoating were: inlet air temperature 70° C., spray air volume about 1200 Nl/min/gun, inlet air volume about 50 Nm³/min, rotor rev rate about 150 rpm, spray rate about 180 mL/min/gun, spray position lower side. The obtained fine granules were then dried at 80° C. for 10 min in the tumbling fluidized bed coater, and then cooled to outlet air temperature 35° C. The obtained mannitol-overcoated controlled release fine granules were passed through a round m sieve to give the outermost layer-coated fine granules with a particle size of 250 μm-425 μm.

[Composition of mannitol-overcoated controlled
release fine granules (83.2 mg)]

| | | |
|---|---|---|
| controlled release fine granules | 80.2209 | mg |
| (Production Example 40) | | |
| mannitol | 2.9791 | mg |
| total | 83.2 | mg |

### Production Example 42

### Production of Controlled Release Fine Granules

Purified water (78.0 kg) was heated to 70° C., and polysorbate 80 (0.8165 kg), glycerol monostearate (2.041 kg), yellow ferric oxide (0.06532 kg) and ferric oxide (0.06532 kg) were dispersed therein. The suspension was cooled to room temperature, and added to methacrylic acid/methyl acrylate/methyl methacrylate copolymer dispersion (Eudragit FS30D) (136.1 kg), triethyl citrate (4.082 kg) and purified water (15.6 kg) and uniformly mixed to give a coating solution. The fine granules coated with intermediate layer (43.74 kg) obtained in Production Example 38 were coated with the aforementioned coating solution (236.7 kg, 5% increased charge amount) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

The thickness of the controlled release film of the obtained fine granules was about 37.7

[Composition of controlled release fine granules (206.838 mg)]

| | | |
|---|---|---|
| fine granules coated with intermediate layer | 101.25 | mg |
| (Production Example 38) | | |
| methacrylic acid/methyl acrylate/methyl | 90.0 | mg |
| methacrylate copolymer | | |
| polysorbate 80 | 1.8 | mg |
| glycerol monostearate | 4.5 | mg |
| triethyl citrate | 9.0 | mg |
| yellow ferric oxide | 0.144 | mg |
| ferric oxide | 0.144 | mg |
| total | 206.838 | mg |

### Production Example 43

### Production of Controlled Release Fine Granules

Purified water (52.8 kg) was heated to 70° C., and polysorbate 80 (0.5535 kg), glycerol monostearate (1.384 kg), yellow ferric oxide (0.04413 kg) and ferric oxide (0.04413 kg) were dispersed therein. The suspension was cooled to room temperature, and then ethyl acrylate/methyl methacrylate copolymer dispersion (Eudragit NE30D) (7.687 kg) and citric acid (0.02315 kg) were added and the mixture was uniformly mixed. Furthermore, methacrylic acid/ethyl acrylate copolymer dispersion (Eudragit L30D-55) (69.19 kg), polyethylene glycol (2.306 kg) and purified water (4.2 kg) were added and the mixture was uniformly mixed to give a coating solution. The controlled release fine granules (89.3 kg) obtained in Production Example 42 were coated with a predetermined amount (17.3 kg, 5% increased charge amount) of the aforementioned coating solution (138.3 kg) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

[Composition of controlled release fine granules
(214.4173 mg) ]

| | | |
|---|---|---|
| controlled release fine granules | 206.838 | mg |
| (Production Example 42) | | |
| methacrylic acid/ethyl acrylate copolymer | 5.738 | mg |
| ethyl acrylate/methyl methacrylate copolymer | 0.6375 | mg |
| polysorbate 80 | 0.153 | mg |
| glycerol monostearate | 0.3825 | mg |
| polyethylene glycol | 0.6375 | mg |
| citric acid | 0.0064 | mg |
| yellow ferric oxide | 0.0122 | mg |
| ferric oxide | 0.0122 | mg |
| total | 214.4173 | mg |

### Production Example 44

### Production of Mannitol-Overcoated Controlled Release Fine Granules

Mannitol (3.757 kg) was dissolved in purified water (22.5 kg) to give a coating solution. The controlled release fine granules (92.6 kg) obtained in Production Example 43 were coated with the aforementioned coating solution (26.3 kg, 5% increased charge amount) by using a tumbling fluidized bed coater (MP-400, manufactured by POWREX Corporation). The coating conditions for mannitol overcoating were: inlet air temperature 55° C., spray air volume about 1200 Nl/min/gun, inlet air volume about 60 Nm³/min, rotor rev rate about 150 rpm, spray rate about 180 mL/min/gun, spray position lower side. The obtained fine granules were then dried at inlet air temperature 80° C. for 15 min in the tumbling fluidized bed coater, and then cooled to outlet air temperature 35° C. The obtained mannitol-overcoated controlled release fine granules were passed through a round sieve to give the outermost layer-coated fine granules with a particle size of 250 μm-425 μm.

[Composition of mannitol-overcoated controlled
release fine granules (222.7 mg)]

| | | |
|---|---|---|
| controlled release fine granules | 214.4173 | mg |
| (Production Example 43) | | |
| mannitol | 8.2827 | mg |
| total | 222.7 | mg |

### Production Example 45

### Production of Controlled Release Fine Granules

Purified water (86.7 kg) was heated to 70° C., and polysorbate 80 (0.9072 kg), glycerol monostearate (2.268 kg), yellow ferric oxide (0.07258 kg) and ferric oxide (0.07258 kg) were dispersed therein. The suspension was cooled to room temperature, and added to methacrylic acid/methyl acrylate/methyl methacrylate copolymer dispersion (Eudragit FS30D) (151.2 kg), triethyl citrate (4.536 kg) and purified water (17.3 kg) and uniformly mixed to give a coating solution. The fine granules coated with intermediate layer (43.74 kg) obtained in Production Example 38 were coated with the aforementioned coating solution (263.1 kg, 5% increased charge amount) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

The thickness of the controlled release film of the obtained fine granules was about 41.0 μm.

[Composition of controlled release fine granules (218.57 mg)]

| | | |
|---|---|---|
| fine granules coated with intermediate layer | 101.25 | mg |
| (Production Example 38) | | |
| methacrylic acid/methyl acrylate/methyl | 100.0 | mg |
| methacrylate copolymer | | |
| polysorbate 80 | 2.0 | mg |
| glycerol monostearate | 5.0 | mg |
| triethyl citrate | 10.0 | mg |
| yellow ferric oxide | 0.16 | mg |
| ferric oxide | 0.16 | mg |
| total | 218.57 | mg |

### Production Example 46

### Production of Controlled Release Fine Granules

Purified water (52.7 kg) was heated to 70° C., and polysorbate 80 (0.5533 kg), glycerol monostearate (1.383 kg), yellow ferric oxide (0.04426 kg) and ferric oxide (0.04426 kg) were dispersed therein. The suspension was cooled to room temperature, and then ethyl acrylate/methyl methacrylate copolymer dispersion (Eudragit NE30D) (7.684 kg) and citric acid (0.02311 kg) were added and the mixture was uniformly mixed. Furthermore, methacrylic acid/ethyl acrylate copolymer dispersion (Eudragit L30D-55) (69.16 kg), polyethylene glycol (2.305 kg) and purified water (4.2 kg) were added and the mixture was uniformly mixed to give a coating solution. The controlled release fine granules (94.4 kg) obtained in Production Example 45 were coated with a predetermined amount (19.3 kg, 5% increased charge amount) of the aforementioned coating solution (138.2 kg) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

[Composition of controlled release fine granules
(226.9909 mg)]

| | | |
|---|---|---|
| controlled release fine granules | 218.57 | mg |
| (Production Example 45) | | |
| methacrylic acid/ethyl acrylate copolymer | 6.375 | mg |
| ethyl acrylate/methyl methacrylate copolymer | 0.7083 | mg |
| polysorbate 80 | 0.17 | mg |
| glycerol monostearate | 0.425 | mg |
| polyethylene glycol | 0.7083 | mg |
| citric acid | 0.0071 | mg |
| yellow ferric oxide | 0.0136 | mg |
| ferric oxide | 0.0136 | mg |
| total | 226.9909 | mg |

### Production Example 47

### Production of Mannitol-Overcoated Controlled Release Fine Granules

Mannitol (3.86 kg) was dissolved in purified water (23.2 kg) to give a coating solution. The controlled release fine granules (98.1 kg) obtained in Production Example 46 were coated with the aforementioned coating solution (27.1 kg, 5% increased charge amount) by using a tumbling fluidized bed coater (MP-400, manufactured by POWREX Corporation). The coating conditions for mannitol overcoating were: inlet air temperature 55° C., spray air volume about 1200 Nl/min/gun, inlet air volume about 60 Nm³/min, rotor rev rate about 150 rpm, spray rate about 180 mL/min/gun, spray position lower side. The obtained fine granules were then dried at inlet air temperature 80° C. for 15 min in the tumbling fluidized bed coater, and then cooled to outlet air temperature 35° C. The obtained mannitol-overcoated controlled release fine granules were passed through a round sieve to give the outermost layer-coated fine granules with a particle size of 250 μm-425 μm.

[Composition of mannitol-overcoated controlled
release fine granules (235.5 mg)]

| | | |
|---|---|---|
| controlled release fine granules | 226.9909 | mg |
| (Production Example 46) | | |
| mannitol | 8.5091 | mg |
| total | 235.5 | mg |

### Production Example 48

### Production of Controlled Release Fine Granules

Purified water (95.3 kg) was heated to 70° C., and polysorbate 80 (0.9979 kg), glycerol monostearate (2.495 kg), yellow ferric oxide (0.07983 kg) and ferric oxide (0.07983 kg) were dispersed therein. The suspension was cooled to room temperature, and added to methacrylic acid/methyl acrylate/methyl methacrylate copolymer dispersion (Eudragit FS30D) (166.3 kg), triethyl citrate (4.99 kg) and purified water (19.1 kg) and uniformly mixed to give a coating solution. The fine granules coated with intermediate layer (43.74 kg), which were obtained in Production Example 38 were coated with the aforementioned coating solution (289.4 kg, 5% increased charge amount) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

The thickness of the controlled release film of the obtained fine granules was about 44.2 μm.

[Composition of controlled release fine granules (230.302 mg)]

| | | |
|---|---|---|
| fine granules coated with intermediate layer | 101.25 | mg |
| (Production Example 38) | | |
| methacrylic acid/methyl acrylate/methyl | 110.0 | mg |
| methacrylate copolymer | | |
| polysorbate 80 | 2.2 | mg |
| glycerol monostearate | 5.5 | mg |
| triethyl citrate | 11 | mg |
| yellow ferric oxide | 0.176 | mg |
| ferric oxide | 0.176 | mg |
| total | 230.302 | mg |

### Production Example 49

### Production of Controlled Release Fine Granules

Purified water (52.8 kg) was heated to 70° C., and polysorbate 80 (0.5514 kg), glycerol monostearate (1.378 kg), yellow ferric oxide (0.04423 kg) and ferric oxide (0.04423 kg) were dispersed therein. The suspension was cooled to room temperature, and then ethyl acrylate/methyl methacrylate copolymer dispersion (Eudragit NE30D) (7.657 kg) and citric acid (0.023 kg) were added and the mixture was uniformly mixed. Furthermore, methacrylic acid/ethyl acrylate copolymer dispersion (Eudragit L30D-55) (68.92 kg), polyethylene glycol (2.297 kg) and purified water (4.2 kg) were added and the mixture was uniformly mixed to give a coating solution. The controlled release fine granules (99.5 kg) obtained in Production Example 48 were coated with a predetermined amount (21.2 kg, 5% increased charge amount) of the aforementioned coating solution (137.9 kg) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

[Composition of controlled release fine granules (239.5655 mg)]

| | | |
|---|---|---|
| controlled release fine granules | 230.302 | mg |
| (Production Example 48) | | |
| methacrylic acid/ethyl acrylate copolymer | 7.013 | mg |
| ethyl acrylate/methyl methacrylate copolymer | 0.7791 | mg |
| polysorbate 80 | 0.187 | mg |
| glycerol monostearate | 0.4675 | mg |
| polyethylene glycol | 0.7791 | mg |
| citric acid | 0.0078 | mg |
| yellow ferric oxide | 0.015 | mg |
| ferric oxide | 0.015 | mg |
| total | 239.5655 | mg |

### Production Example 50

### Production of Mannitol-Overcoated Controlled Release Fine Granules

Mannitol (3.962 kg) was dissolved in purified water (23.8 kg) to give a coating solution. The controlled release fine granules (103.5 kg) obtained in Production Example 49 were coated with the aforementioned coating solution (27.8 kg, 5% increased charge amount) by using a tumbling fluidized bed coater (MP-400, manufactured by POWREX Corporation). The coating conditions for mannitol overcoating were: inlet air temperature 55° C., spray air volume about 1200 Nl/min/gun, inlet air volume about 60 Nm³/min, rotor rev rate about 150 rpm, spray rate about 180 ml/min/gun, spray position lower side. The obtained fine granules were then dried at inlet air temperature 80° C. for 15 min in the tumbling fluidized bed coater, and then cooled to outlet air temperature 35° C. The obtained mannitol-overcoated controlled release fine granules were passed through a round sieve to give the outermost layer-coated fine granules with a particle size of 250 μm-425 μm.

[Composition of mannitol-overcoated controlled release fine
granules (248.3 mg)]

| | | |
|---|---|---|
| controlled release fine granules | 239.5655 | mg |
| (Production Example 49) | | |
| mannitol | 8.7345 | mg |
| total | 248.3 | mg |

### Production Example 51

### Production of Outer Layer Component-Granulated Powder

Mannitol (40810 g), low-substituted hydroxypropyl cellulose (L-HPC-33, 7168 g), crystalline cellulose (7168 g) and crospovidone (3584 g) were charged in a fluidized bed granulator (FD-WSG-60, manufactured by POWREX CORPORATION), and they were granulated by spraying an aqueous solution of mannitol (3584 g) and citric acid (716.8 g) in purified water (19900 g) and dried to give a granulated powder.

[Composition of outer layer component-granulated powder
(315.156 mg)]

| | | |
|---|---|---|
| mannitol | 221.972 | mg |
| low-substituted hydroxypropyl cellulose | 35.84 | mg |
| crospovidone | 17.92 | mg |
| crystalline cellulose | 35.84 | mg |
| citric acid | 3.584 | mg |
| total | 315.156 | mg |

### Production Example 52

### Production of Outer Layer Component-Granulated Powder

Mannitol (41530 g), low-substituted hydroxypropyl cellulose (L-HPC-33, 7241 g), crystalline cellulose (7241 g) and crospovidone (3621 g) were charged in a fluidized bed granulator (FD-WSG-60, manufactured by POWREX CORPORATION), and they were granulated by spraying an aqueous solution of mannitol (3621 g) and citric acid (724.1 g) in purified water (19940 g) and dried to give a granulated powder.

[Composition of outer layer component-granulated powder
(331.468 mg)]

| | | |
|---|---|---|
| mannitol | 233.916 | mg |
| low-substituted hydroxypropyl cellulose | 37.52 | mg |
| crospovidone | 18.76 | mg |
| crystalline cellulose | 37.52 | mg |
| citric acid | 3.752 | mg |
| total | 331.468 | mg |

### Production Example 53

### Production of Outer Layer Component-Granulated Powder

Mannitol (42080 g), low-substituted hydroxypropyl cellulose (L-HPC-33, 7291 g), crystalline cellulose (7291 g) and crospovidone (3646 g) were charged in a fluidized bed granulator (FD-WSG-60, manufactured by POWREX CORPORATION), and they were granulated by spraying an aqueous solution of mannitol (3646 g) and citric acid (729.1 g) in purified water (19940 g) and dried to give a granulated powder.

[Composition of outer layer component-granulated powder
(347.78 mg)]

| | | |
|---|---|---|
| mannitol | 245.86 | mg |
| low-substituted hydroxypropyl cellulose | 39.2 | mg |
| crospovidone | 19.6 | mg |
| crystalline cellulose | 39.2 | mg |
| citric acid | 3.92 | mg |
| total | 347.78 | mg |

### Example 5

### Production of Orally Disintegrating Tablet

The mannitol-coated fine granules (15640 g) obtained in Production Example 41, the mannitol-coated fine granules (41870 g) obtained in Production Example 44, the outer layer component-granulated powder (59250 g) obtained in Production Example 51, sucralose (1925 g), flavor (STRAWBERRY DURAROME) (673.8 g) and magnesium stearate (962.6 g) were mixed by using a tumbling mixer (TM-400S, SHOWA KAGAKU KIKAI CO., LTD.) to give a mixed powder. The obtained mixed powder (120300 g) was tableted by using a rotary tableting machine (AQU31029SW4JII (Roman number), Kikusui Seisakusho Ltd.) (640 mg/tablet, a 13 mmφ punch, flat-faced with beveled edge, tableting pressure 26.0 kN) to give the orally disintegrating tablet (640 mg) containing compound X (30 mg) of the present invention.

[Composition of orally disintegrating tablet (640 mg)]

| | | |
|---|---|---|
| mannitol-coated fine granules | 83.2 | mg |
| (Production Example 41) | | |
| mannitol-coated fine granules | 222.7 | mg |
| (Production Example 44) | | |
| outer layer component-granulated powder | 315.156 | mg |
| (Production Example 51) | | |
| sucralose | 10.24 | mg |
| flavor | 3.584 | mg |
| magnesium stearate | 5.12 | mg |
| total | 640 | mg |

The hardness and the disintegration time of the obtained tablet were 45 N and 30 seconds, respectively. The dissolution rate of the obtained tablet in 0.1N HCl in 1 hour was 1.2%, showing superior acid resistance.

### Example 6

### Production of Orally Disintegrating Tablet

The mannitol-coated fine granules (14980 g) obtained in Production Example 41, the mannitol-coated fine granules (42390 g) obtained in Production Example 47, the outer layer component-granulated powder (59660 g) obtained in Production Example 52, sucralose (1930 g), flavor (STRAWBERRY DURAROME) (675.4 g) and magnesium stearate (964.8 g) were mixed by using a tumbling mixer (TM-400S, SHOWA KAGAKU KIKAI CO., LTD.) to give a mixed powder. The obtained mixed powder (120600 g) was tableted by using a rotary tableting machine (AQU31029SW4JII (Roman number), Kikusui Seisakusho Ltd.) (670 mg/tablet, a 13 mmφ punch, flat-faced with beveled edge, tableting pressure 27.0 kN) to give the orally disintegrating tablet (670 mg) containing compound X (30 mg) of the present invention.

[Composition of orally disintegrating tablet (670 mg)]

| | | |
|---|---|---|
| mannitol-coated fine granules | 83.2 | mg |
| (Production Example 41) | | |
| mannitol-coated fine granules | 235.5 | mg |
| (Production Example 47) | | |
| outer layer component-granulated powder | 331.468 | mg |
| (Production Example 52) | | |
| sucralose | 10.72 | mg |
| flavor | 3.752 | mg |
| magnesium stearate | 5.36 | mg |
| total | 670 | mg |

The hardness and the disintegration time of the obtained tablet were 46 N and 30 seconds, respectively. The dissolution rate of the obtained tablet in 0.1N HCl in 1 hour was 1.1%, showing superior acid resistance.

### Example 7

### Production of Orally Disintegrating Tablet

The mannitol-coated fine granules (14310 g) obtained in Production Example 41, the mannitol-coated fine granules (42710 g) obtained in Production Example 50, the outer layer component-granulated powder (59820 g) obtained in Production Example 53, sucralose (1926 g), flavor (STRAWBERRY DURAROME) (674.2 g) and magnesium stearate (963.2 g) were mixed by using a tumbling mixer (TM-400S, SHOWA KAGAKU KIKAI CO., LTD.) to give a mixed powder. The obtained mixed powder (120400 g) was tableted by using a rotary tableting machine (AQU31029SW4JII (Roman number), Kikusui Seisakusho Ltd.) (700 mg/tablet, a 13 mmφ punch, flat-faced with beveled edge, tableting pressure 27.0 kN) to give the orally disintegrating tablet (700 mg) containing compound X (30 mg) of the present invention.

[Composition of orally disintegrating tablet (700 mg)]

| | | |
|---|---|---|
| mannitol-coated fine granules | 83.2 | mg |
| (Production Example 41) | | |
| mannitol-coated fine granules | 248.3 | mg |
| (Production Example 50) | | |
| outer layer component-granulated powder | 347.78 | mg |
| (Production Example 53) | | |
| sucralose | 11.2 | mg |
| flavor | 3.92 | mg |
| magnesium stearate | 5.6 | mg |
| total | 700 | mg |

The hardness and the disintegration time of the obtained tablet were 45 N and 30 seconds, respectively. The dissolution rate of the obtained tablet in 0.1N HCl in 1 hour was 1.1%, showing superior acid resistance.

### Production Example 54

### Production of Controlled Release Fine Granules

Purified water (2267.8 g) was heated to 80° C., and polysorbate 80 (19.22 g), glycerol monostearate (48.06 g), triethyl citrate (96.12 g), yellow ferric oxide (1.538 g) and ferric oxide (1.538 g) were dispersed therein. The suspension was cooled to room temperature, and added to methacrylic acid/methyl acrylate/methyl methacrylate copolymer dispersion (Eudragit FS30D) (3204 g) and uniformly mixed to give a coating solution. The fine granules coated with intermediate layer (901.1 g) obtained in Production Example 3 were coated with a predetermined amount (4934 g, 5% increased charge amount) of the aforementioned coating solution (5638 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 37.7 μm.

[Composition of controlled release fine granules (206.838 mg)]

| | | |
|---|---|---|
| fine granules coated with intermediate layer | 101.25 | mg |
| (Production Example 3) | | |
| methacrylic acid/methyl acrylate/methyl | 90.0 | mg |
| methacrylate copolymer | | |
| polysorbate 80 | 1.8 | mg |
| glycerol monostearate | 4.5 | mg |
| triethyl citrate | 9.0 | mg |
| yellow ferric oxide | 0.144 | mg |
| ferric oxide | 0.144 | mg |
| total | 206.838 | mg |

### Production Example 55

### Production of Fine Granules Coated with Intermediate Layer

The intermediate layer coating solution was produced by dissolving hypromellose (TC-5E, 156.996 g) and mannitol (156.996 g) in purified water (1682.1 g), and dispersing titanium oxide (67.284 g), talc (67.284 g) and low-substituted hydroxypropyl cellulose (L-HPC-32, 112.14 g) in the obtained solution. The controlled release fine granules (1840.8 g) obtained in Production Example 54 were coated with a predetermined amount (280.35 g) of the intermediate layer coating solution (2242.8 g) by using a tumbling fluidized bed coater (MP-10 TOKU-2 type, manufactured by POWREX CORPORATION).

The coating conditions were: inlet air temperature about 45° C., spray air pressure about 0.35 MPa, spray air volume about 100 Nl/min, inlet air volume about 1.5 m³/min, rotor rev rate about 550 rpm, spray rate about 10 g/min, spray position lower side. After the completion of coating, the obtained fine granules were then dried at 85° C. for about 40 min in the tumbling fluidized bed coater, and passed through a round sieve to give fine granules coated with intermediate layer with a particle size of 250 μm-425 μm.

[Composition of fine granules coated with intermediate layer
(214.713 mg)]

| | | |
|---|---|---|
| controlled release fine granules | 206.838 | mg |
| (Production Example 54) | | |
| hypromellose | 2.205 | mg |
| low-substituted hydroxypropyl cellulose | 1.575 | mg |
| talc | 0.945 | mg |
| titanium oxide | 0.945 | mg |
| mannitol | 2.205 | mg |
| total | 214.713 | mg |

### Production Example 56

### Production of Controlled Release Fine Granules

Purified water (1188.9 g) was heated to 80° C., and polysorbate 80 (10.08 g), glycerol monostearate (25.2 g), triethyl citrate (50.4 g), yellow ferric oxide (0.806 g) and ferric oxide (0.806 g) were dispersed therein. The suspension was cooled to room temperature, and added to methacrylic acid/methyl acrylate/methyl methacrylate copolymer dispersion (Eudragit FS30D) (1680 g) and uniformly mixed to give a coating solution. The fine granules coated with intermediate layer (1288 g) obtained in Production Example 55 were coated with a predetermined amount (370 g, 5% increased charge amount) of the aforementioned coating solution (2956 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the film containing methacrylic acid/methyl acrylate/methyl methacrylate copolymer of the obtained fine granules was about 41.0 μm.

[Composition of controlled release fine granules (226.445 mg)]

| | | |
|---|---|---|
| fine granules coated with intermediate layer | 214.713 | mg |
| (Production Example 55) | | |
| methacrylic acid/methyl acrylate/methyl | 10 | mg |
| methacrylate copolymer | | |
| polysorbate 80 | 0.2 | mg |
| glycerol monostearate | 0.5 | mg |
| triethyl citrate | 1 | mg |
| yellow ferric oxide | 0.016 | mg |
| ferric oxide | 0.016 | mg |
| total | 226.445 | mg |

### Production Example 57

### Production of Controlled Release Fine Granules

Purified water (1188.9 g) was heated to 80° C., and polysorbate 80 (10.08 g), glycerol monostearate (25.2 g), triethyl citrate (50.4 g), yellow ferric oxide (0.806 g) and ferric oxide (0.806 g) were dispersed therein. The suspension was cooled to room temperature, and added to methacrylic acid/methyl acrylate/methyl methacrylate copolymer dispersion (Eudragit FS30D) (1680 g) and uniformly mixed to give a coating solution. The fine granules coated with intermediate layer (1288 g) obtained in Production Example 55 were coated with a predetermined amount (739 g, 5% increased charge amount) of the aforementioned coating solution (2956 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 44.1 μm.

[Composition of controlled release fine granules (238.177 mg)]

| | | |
|---|---|---|
| fine granules coated with intermediate layer | 214.713 | mg |
| (Production Example 55) | | |
| methacrylic acid/methyl acrylate/methyl | 20 | mg |
| methacrylate copolymer | | |
| polysorbate 80 | 0.4 | mg |
| glycerol monostearate | 1 | mg |
| triethyl citrate | 2 | mg |
| yellow ferric oxide | 0.032 | mg |
| ferric oxide | 0.032 | mg |
| total | 238.177 | mg |

### Production Example 58

### Production of Controlled Release Fine Granules

Purified water (1188.9 g) was heated to 80° C., and polysorbate 80 (10.08 g), glycerol monostearate (25.2 g), triethyl citrate (50.4 g), yellow ferric oxide (0.806 g) and ferric oxide (0.806 g) were dispersed therein. The suspension was cooled to room temperature, and added to methacrylic acid/methyl acrylate/methyl methacrylate copolymer dispersion (Eudragit FS30D) (1680 g) and uniformly mixed to give a coating solution. The fine granules coated with intermediate layer (1288 g) obtained in Production Example 55 were coated with a predetermined amount (1109 g, 5% increased charge amount) of the aforementioned coating solution (2956 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 47.1 μm.

[Composition of controlled release fine granules (249.909 mg)]

| | | |
|---|---|---|
| fine granules coated with intermediate layer | 214.713 | mg |
| (Production Example 55) | | |
| methacrylic acid/methyl acrylate/methyl | 30 | mg |
| methacrylate copolymer | | |
| polysorbate 80 | 0.6 | mg |
| glycerol monostearate | 1.5 | mg |
| triethyl citrate | 3 | mg |
| yellow ferric oxide | 0.048 | mg |
| ferric oxide | 0.048 | mg |
| total | 249.909 | mg |

### Production Example 59

### Production of Controlled Release Fine Granules

Purified water (1188.9 g) was heated to 80° C., and polysorbate 80 (10.08 g), glycerol monostearate (25.2 g), triethyl citrate (50.4 g), yellow ferric oxide (0.806 g) and ferric oxide (0.806 g) were dispersed therein. The suspension was cooled to room temperature, and added to methacrylic acid/methyl acrylate/methyl methacrylate copolymer dispersion (Eudragit FS30D) (1680 g) and uniformly mixed to give a coating solution. The fine granules coated with intermediate layer (1288 g) obtained in Production Example 55 were coated with a predetermined amount (1478 g, 5% increased charge amount) of the aforementioned coating solution (2956 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 50.0 μm.

[Composition of controlled release fine granules (261.641 mg)]

| | | |
|---|---|---|
| fine granules coated with intermediate layer | 214.713 | mg |
| (Production Example 55) | | |
| methacrylic acid/methyl acrylate/methyl | 40 | mg |
| methacrylate copolymer | | |
| polysorbate 80 | 0.8 | mg |
| glycerol monostearate | 2 | mg |
| triethyl citrate | 4 | mg |
| yellow ferric oxide | 0.064 | mg |
| ferric oxide | 0.064 | mg |
| total | 261.641 | mg |

### Production Example 60

### Production of Controlled Release Fine Granules

Purified water (1188.9 g) was heated to 80° C., and polysorbate 80 (10.08 g), glycerol monostearate (25.2 g), triethyl citrate (50.4 g), yellow ferric oxide (0.806 g) and ferric oxide (0.806 g) were dispersed therein. The suspension was cooled to room temperature, and added to methacrylic acid/methyl acrylate/methyl methacrylate copolymer dispersion (Eudragit FS30D) (1680 g) and uniformly mixed to give a coating solution. The fine granules coated with intermediate layer (1288 g) obtained in Production Example 55 were coated with a predetermined amount (1848 g, 5% increased charge amount) of the aforementioned coating solution (2956 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 52.9 μm.

[Composition of controlled release fine granules (273.373 mg)]

| | | |
|---|---|---|
| fine granules coated with intermediate layer | 214.713 | mg |
| (Production Example 55) | | |
| methacrylic acid/methyl acrylate/methyl | 50 | mg |
| methacrylate copolymer | | |
| polysorbate 80 | 1 | mg |
| glycerol monostearate | 2.5 | mg |
| triethyl citrate | 5 | mg |
| yellow ferric oxide | 0.08 | mg |
| ferric oxide | 0.08 | mg |
| total | 273.373 | mg |

### Production Example 61

### Production of Controlled Release Fine Granules

Purified water (1188.9 g) was heated to 80° C., and polysorbate 80 (10.08 g), glycerol monostearate (25.2 g), triethyl citrate (50.4 g), yellow ferric oxide (0.806 g) and ferric oxide (0.806 g) were dispersed therein. The suspension was cooled to room temperature, and added to methacrylic acid/methyl acrylate/methyl methacrylate copolymer dispersion (Eudragit FS30D) (1680 g) and uniformly mixed to give a coating solution. The fine granules coated with intermediate layer (1288 g) obtained in Production Example 55 were coated with a predetermined amount (2217 g, 5% increased charge amount) of the aforementioned coating solution (2956 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 55.6 μm.

[Composition of controlled release fine granules (285.105 mg)]

| | | |
|---|---|---|
| fine granules coated with intermediate layer | 214.713 | mg |
| (Production Example 55) | | |
| methacrylic acid/methyl acrylate/methyl | 60 | mg |
| methacrylate copolymer | | |
| polysorbate 80 | 1.2 | mg |
| glycerol monostearate | 3 | mg |
| triethyl citrate | 6 | mg |
| yellow ferric oxide | 0.096 | mg |
| ferric oxide | 0.096 | mg |
| total | 285.105 | mg |

### Production Example 62

### Production of Controlled Release Fine Granules

Purified water (1188.9 g) was heated to 80° C., and polysorbate 80 (10.08 g), glycerol monostearate (25.2 g), triethyl citrate (50.4 g), yellow ferric oxide (0.806 g) and ferric oxide (0.806 g) were dispersed therein. The suspension was cooled to room temperature, and added to methacrylic acid/methyl acrylate/methyl methacrylate copolymer dispersion (Eudragit FS30D) (1680 g) and uniformly mixed to give a coating solution. The fine granules coated with intermediate layer (1288 g) obtained in Production Example 55 were coated with a predetermined amount (2587 g, 5% increased charge amount) of the aforementioned coating solution (2956 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.2 m³/min, rotor rev rate about 550 rpm, spray rate about 10 g/min, spray position lower side.

The thickness of the controlled release film of the obtained fine granules was about 58.3 μm.

[Composition of controlled release fine granules (296.837 mg)]

| | | |
|---|---|---|
| fine granules coated with intermediate layer | 214.713 | mg |
| (Production Example 55) | | |
| methacrylic acid/methyl acrylate/methyl | 70 | mg |
| methacrylate copolymer | | |
| polysorbate 80 | 1.4 | mg |
| glycerol monostearate | 3.5 | mg |
| triethyl citrate | 7 | mg |
| yellow ferric oxide | 0.112 | mg |
| ferric oxide | 0.112 | mg |
| total | 296.837 | mg |

### Production Example 63

### Production of Controlled Release Fine Granules

Purified water (494.11 g) was heated to 80° C., and polysorbate 80 (4.896 g), glycerol monostearate (12.24 g), polyethylene glycol (20.4 g), yellow ferric oxide (0.3917 g) and ferric oxide (0.3917 g) were dispersed therein. The suspension was cooled to room temperature, and then ethyl acrylate/methyl methacrylate copolymer dispersion (Eudragit NE30D) (68 g) and citric acid (0.2045 g) were added and the mixture was uniformly mixed. Furthermore, methacrylic acid/ethyl acrylate copolymer dispersion (Eudragit L30D-55) (612 g) was added and the mixture was uniformly mixed to give a coating solution. The controlled release fine granules (1781 g) obtained in Production Example 62 were coated with a predetermined amount (438 g, 5% increased charge amount) of the aforementioned coating solution (1213 g) by using a tumbling fluidized bed coater (MP-10 TOKU-2 type, manufactured by POWREX CORPORATION). The coating conditions were: inlet air temperature 45° C., spray air pressure about 0.45 MPa, spray air volume about 120 Nl/min, inlet air volume about 1.5 m³/min, rotor rev rate about 600 rpm, spray rate about 10 g/min, spray position lower side.

[Composition of controlled release fine granules (310.7315 mg)]

| | | |
|---|---|---|
| controlled release fine granules | 296.837 | mg |
| (Production Example 62) | | |
| methacrylic acid/ethyl acrylate copolymer | 10.51875 | mg |
| ethyl acrylate/methyl methacrylate copolymer | 1.168695 | mg |
| polysorbate 80 | 0.2805 | mg |
| glycerol monostearate | 0.70125 | mg |
| polyethylene glycol | 1.168695 | mg |
| citric acid | 0.011715 | mg |
| yellow ferric oxide | 0.02244 | mg |
| ferric oxide | 0.02244 | mg |
| total | 310.7315 | mg |

### Production Example 64

### Production of Mannitol-Overcoated Controlled Release Fine Granules

Mannitol (99.2 g) was dissolved in purified water (595.3 g) to give a coating solution. The controlled release fine granules (1864 g) obtained in Production Example 63 were coated with a predetermined amount (427 g, 5% increased charge amount) of the aforementioned coating solution (694.5 g) by using a tumbling fluidized bed coater (MP-10 TOKU-2 type, manufactured by POWREX CORPORATION). The coating conditions for mannitol overcoating were: inlet air temperature 70° C., spray air pressure about 0.45 MPa, spray air volume about 120 Nl/min, inlet air volume about 1.5 m³/min, rotor rev rate about 600 rpm, spray rate about 12 g/min, spray position lower side. The obtained fine granules were then dried at 85° C. for about 40 min in the tumbling fluidized bed coater to give the outermost layer-coated fine granules.

[Composition of mannitol-overcoated controlled release fine
granules (320.9 mg)]

| | | |
|---|---|---|
| controlled release fine granules | 310.7315 | mg |
| (Production Example 63) | | |
| mannitol | 10.1685 | mg |
| total | 320.9 | mg |

### Experimental Example 16

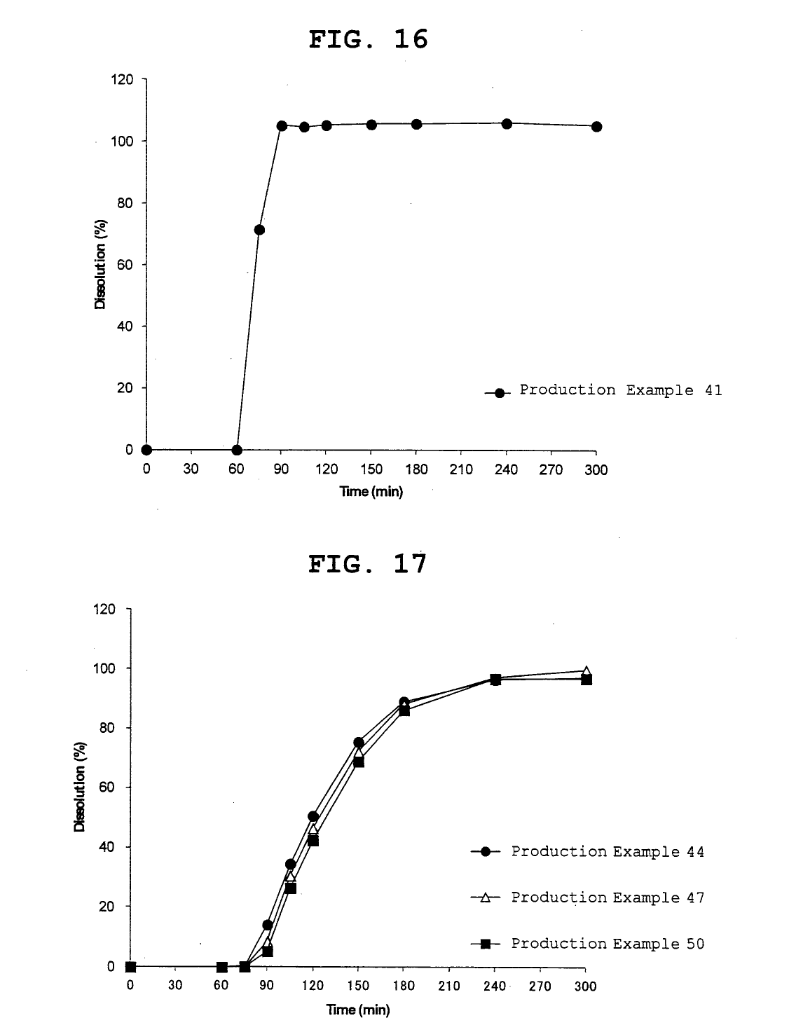

A dissolution test (test method (1)) was performed for the fine granules obtained in Production Example 41. The results are shown in FIG. 16.

### Experimental Example 17

A dissolution test (test method (2)) was performed for the fine granules and granules obtained in Production Examples 44, 47 and 50. The results are shown in FIG. 17.

### Experimental Example 18

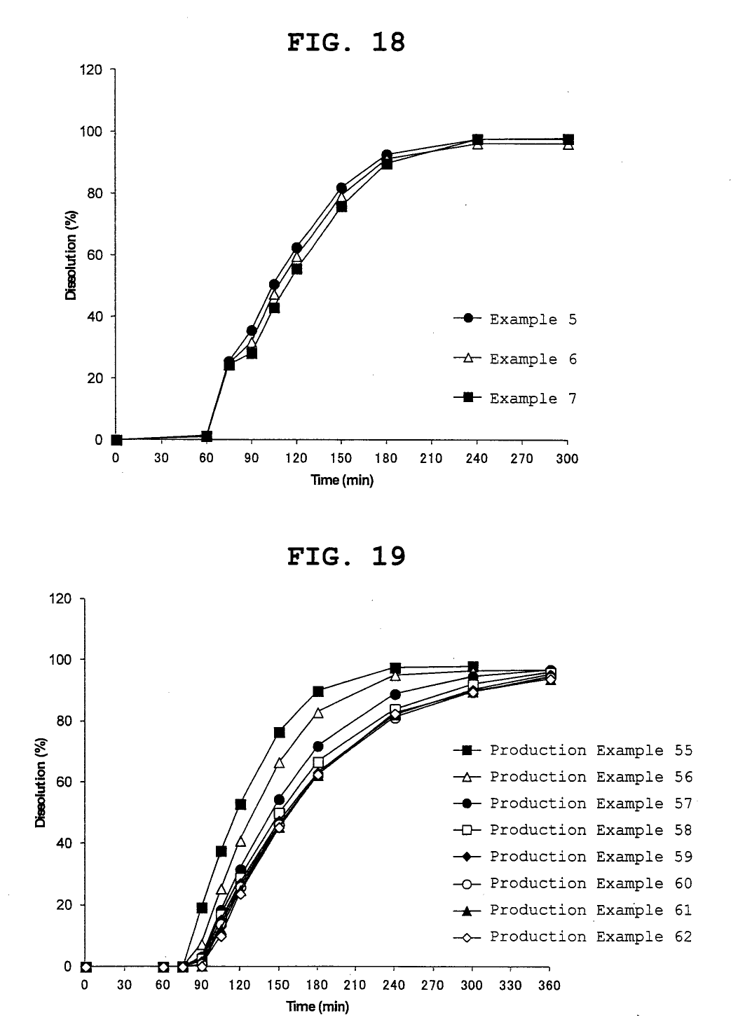

A dissolution test (test method (2)) was performed for the formulations obtained in Examples 5, 6 and 7. The results are shown in FIG. 18.

### Experimental Example 19

A dissolution test (test method (2)) was performed for the fine granules obtained in Production Examples 55, 56, 57, 58, 59, 60, 61 and 62. The results are shown in FIG. 19.

### Experimental Example 20

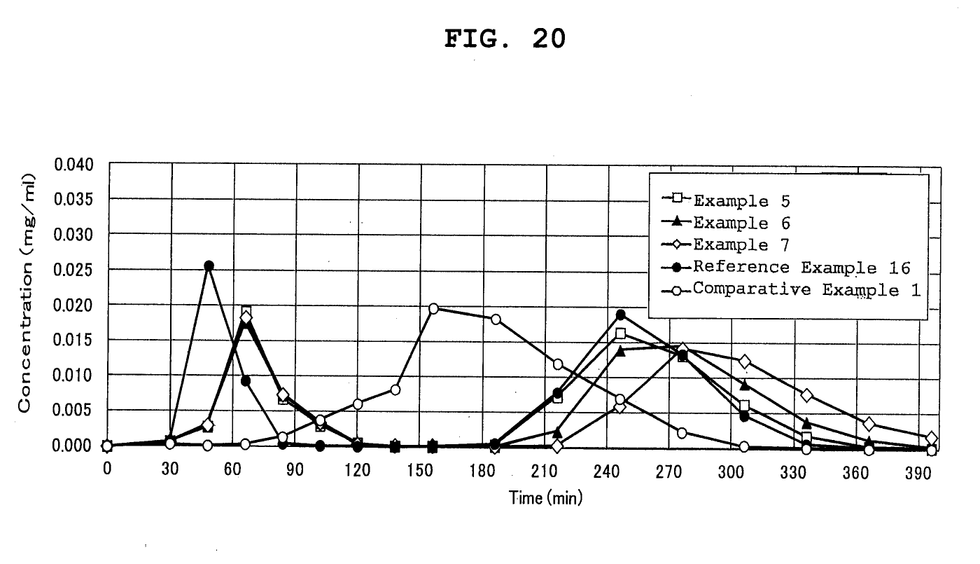

A dissolution test (test method (3)) was performed for the formulations obtained in Examples 5, 6 and 7 and Comparative Example 1 and Reference Example 16. The results are shown in FIG. 20.

### Production Example 65

### Production of Fine Granules Coated with Intermediate Layer

The intermediate layer coating solution was produced by dissolving hypromellose (TC-5E, 4.763 kg) and mannitol (4.763 kg) in purified water (51 kg), and dispersing titanium oxide (2.041 kg), talc (2.041 kg) and low-substituted hydroxypropyl cellulose (L-HPC-32, 3.402 kg) in the obtained solution. The controlled release fine granules (89.4 g) obtained in Production Example 42 were coated with a predetermined amount (13.61 kg) of the intermediate layer coating solution (68 kg) by using a tumbling fluidized bed coater (MP-400, manufactured by POWREX CORPORATION). The coating conditions were: inlet air temperature about 48° C., spray air volume about 1200 Nl/min/gun, inlet air volume about 60 Nm³/min, rotor rev rate about 150 rpm, spray rate about 180 mL/min/gun, spray position lower side. After the completion of coating, and the granules were then dried at 80° C. for about 15 min in the tumbling fluidized bed coater to give fine granules coated with intermediate layer.

[Composition of fine granules coated with intermediate layer
(214.713 mg)]

| | | |
|---|---|---|
| controlled release fine granules | 206.838 | mg |
| (Production Example 42) | | |
| hypromellose | 2.205 | mg |
| low-substituted hydroxypropyl cellulose | 1.575 | mg |
| talc | 0.945 | mg |
| titanium oxide | 0.945 | mg |
| mannitol | 2.205 | mg |
| total | 214.713 | mg |

### Production Example 66

### Production of Controlled Release Fine Granules

Purified water (70.0 kg) was heated to 70° C., and polysorbate 80 (0.7327 kg), glycerol monostearate (1.832 kg), yellow ferric oxide (0.05861 kg) and ferric oxide (0.05861 kg) were dispersed therein. The suspension (72.68 kg) was cooled to room temperature, and a predetermined amount (28.79 kg) was added to methacrylic acid/methyl acrylate/methyl methacrylate copolymer dispersion (Eudragit FS30D) (48.38 kg), triethyl citrate (1.452 kg) and purified water (5.5 kg) and the mixture was uniformly mixed to give a coating solution. The fine granules coated with intermediate layer (74.2 kg) obtained in Production Example 65 were coated with the aforementioned coating solution (84.1 kg, 5% increased charge amount) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

The thickness of the controlled release film of the obtained fine granules was about 50.0

[Composition of controlled release fine granules (261.641 mg)]

| | | |
|---|---|---|
| fine granules coated with intermediate layer | 214.713 | mg |
| (Production Example 65) | | |
| methacrylic acid/methyl acrylate/methyl | 40 | mg |
| methacrylate copolymer | | |
| polysorbate 80 | 0.8 | mg |
| glycerol monostearate | 2 | mg |
| triethyl citrate | 4 | mg |
| yellow ferric oxide | 0.064 | mg |
| ferric oxide | 0.064 | mg |
| total | 261.641 | mg |

### Production Example 67

### Production of Controlled Release Fine Granules

Purified water (54.7 kg) was heated to 70° C., and polysorbate 80 (0.5728 kg), glycerol monostearate (1.432 kg), yellow ferric oxide (0.04583 kg) and ferric oxide (0.04583 kg) were dispersed therein. The suspension was cooled to room temperature, and then ethyl acrylate/methyl methacrylate copolymer dispersion (Eudragit NE30D) (7.956 kg) and citric acid (0.02392 kg) were added and the mixture was uniformly mixed. Furthermore, methacrylic acid/ethyl acrylate copolymer dispersion (Eudragit L30D-55) (71.6 kg), polyethylene glycol (2.387 kg) and purified water (4.4 kg) were added and the mixture was uniformly mixed to give a coating solution. The controlled release fine granules (90.4 kg) obtained in Production Example 66 were coated with predetermined amount (23.13 kg, 5% increased charge amount) of the aforementioned coating solution (143.2 kg) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

[Composition of controlled release fine granules (274.2724 mg)]

| | | |
|---|---|---|
| controlled release fine granules | 261.641 | mg |
| (Production Example 66) | | |
| methacrylic acid/ethyl acrylate copolymer | 9.5625 | mg |
| ethyl acrylate/methyl methacrylate copolymer | 1.06245 | mg |
| polysorbate 80 | 0.255 | mg |
| glycerol monostearate | 0.6375 | mg |
| polyethylene glycol | 1.06245 | mg |
| citric acid | 0.01065 | mg |
| yellow ferric oxide | 0.0204 | mg |
| ferric oxide | 0.0204 | mg |
| total | 274.2724 | mg |

### Production Example 68

### Production of Mannitol-Overcoated Controlled Release Fine Granules

Mannitol (3.421 kg) was dissolved in purified water (20.5 kg) to give a coating solution. The controlled release fine granules (94.8 kg) obtained in Production Example 67 were coated with the aforementioned coating solution (23.92 kg, 5% increased charge amount) by using a tumbling fluidized bed coater (MP-400, manufactured by POWREX Corporation). The coating conditions for mannitol overcoating were: inlet air temperature 55° C., spray air volume about 1200 Nl/min/gun, inlet air volume about 60 Nm³/min, rotor rev rate about 150 rpm, spray rate about 180 mL/min/gun, spray position lower side. The obtained fine granules were then dried at inlet air temperature 80° C. for 15 min in the tumbling fluidized bed coater, and then cooled to outlet air temperature 35° C.

[Composition of mannitol-overcoated controlled release fine
granules (283.7 mg)]

| | | |
|---|---|---|
| controlled release fine granules | 274.2724 | mg |
| (Production Example 67) | | |
| mannitol | 9.4276 | mg |
| total | 283.7 | mg |

### Production Example 69

### Production of Controlled Release Fine Granules

Purified water (71.3 kg) was heated to 70° C., and polysorbate 80 (0.7465 kg), glycerol monostearate (1.866 kg), yellow ferric oxide (0.05972 kg) and ferric oxide (0.05972 kg) were dispersed therein. The suspension (74.03 kg) was cooled to room temperature, and a predetermined amount (43.19 kg) was added to methacrylic acid/methyl acrylate/methyl methacrylate copolymer dispersion (Eudragit FS30D) (72.58 kg), triethyl citrate (2.177 kg) and purified water (8.3 kg) and the mixture was uniformly mixed to give a coating solution. The fine granules coated with intermediate layer (74.2 kg) obtained in Production Example 65 were coated with the aforementioned coating solution (126.2 kg, 5% increased charge amount) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

The thickness of the controlled release film of the obtained fine granules was about 55.6 μm.

[Composition of controlled release fine granules (285.105 mg)]

| | | |
|---|---|---|
| fine granules coated with intermediate layer | 214.713 | mg |
| (Production Example 65) | | |
| methacrylic acid/methyl acrylate/methyl | 60 | mg |
| methacrylate copolymer | | |
| polysorbate 80 | 1.2 | mg |
| glycerol monostearate | 3 | mg |
| triethyl citrate | 6 | mg |
| yellow ferric oxide | 0.096 | mg |
| ferric oxide | 0.096 | mg |
| total | 285.105 | mg |

### Production Example 70

### Production of Controlled Release Fine Granules

Purified water (53.9 kg) was heated to 70° C., and polysorbate 80 (0.5640 kg), glycerol monostearate (1.410 kg), yellow ferric oxide (0.04512 kg) and ferric oxide (0.04512 kg) were dispersed therein. The suspension was cooled to room temperature, and then ethyl acrylate/methyl methacrylate copolymer dispersion (Eudragit NE30D) (7.833 kg) and citric acid (0.02356 kg) were added and the mixture was uniformly mixed. Furthermore, methacrylic acid/ethyl acrylate copolymer dispersion (Eudragit L30D-55) (70.5 kg), polyethylene glycol (2.350 kg) and purified water (4.3 kg) were added and the mixture was uniformly mixed to give a coating solution. The controlled release fine granules (98.5 kg) obtained in Production Example 69 were coated with a predetermined amount (24.67 kg, 5% increased charge amount) of the aforementioned coating solution (141.0 kg) by using a tumbling fluidized bed coater (MP-400, manufactured by POWREX CORPORATION). The coating conditions were: inlet air temperature 48° C., spray air volume about 1200 Nl/min/gun, inlet air volume about 60 Nm³/min, rotor rev rate about 150 rpm, spray rate about 180 mL/min/gun, spray position lower side.

[Composition of controlled release fine granules (298.5784 mg)]

| | | |
|---|---|---|
| controlled release fine granules | 285.105 | mg |
| (Production Example 69) | | |
| methacrylic acid/ethyl acrylate copolymer | 10.2 | mg |
| ethyl acrylate/methyl methacrylate copolymer | 1.13328 | mg |
| polysorbate 80 | 0.272 | mg |
| glycerol monostearate | 0.68 | mg |
| polyethylene glycol | 1.13328 | mg |
| citric acid | 0.01136 | mg |
| yellow ferric oxide | 0.02176 | mg |
| ferric oxide | 0.02176 | mg |
| total | 298.5784 | mg |

### Production Example 71

### Production of Mannitol-Overcoated Controlled Release Fine Granules

Mannitol (3.6 kg) was dissolved in purified water (21.6 kg) to give a coating solution. The controlled release fine granules (103.2 kg) obtained in Production Example 70 were coated with the aforementioned coating solution (25.2 kg, 5% increased charge amount) by using a tumbling fluidized bed coater (MP-400, manufactured by POWREX Corporation). The coating conditions for mannitol overcoating were: inlet air temperature 55° C., spray air volume about 1200 Nl/min/gun, inlet air volume about 60 Nm³/min, rotor rev rate about 150 rpm, spray rate about 180 ml/min/gun, spray position lower side. The obtained fine granules were then dried at inlet air temperature 80° C. for about 15 min in the tumbling fluidized bed coater, and then cooled to outlet air temperature 35° C.

[Composition of mannitol-overcoated controlled release fine
granules (308.5 mg)]

| | | |
|---|---|---|
| controlled release fine granules | 298.5784 | mg |
| (Production Example 70) | | |
| mannitol | 9.9216 | mg |
| total | 308.5 | mg |

### Experimental Example 21

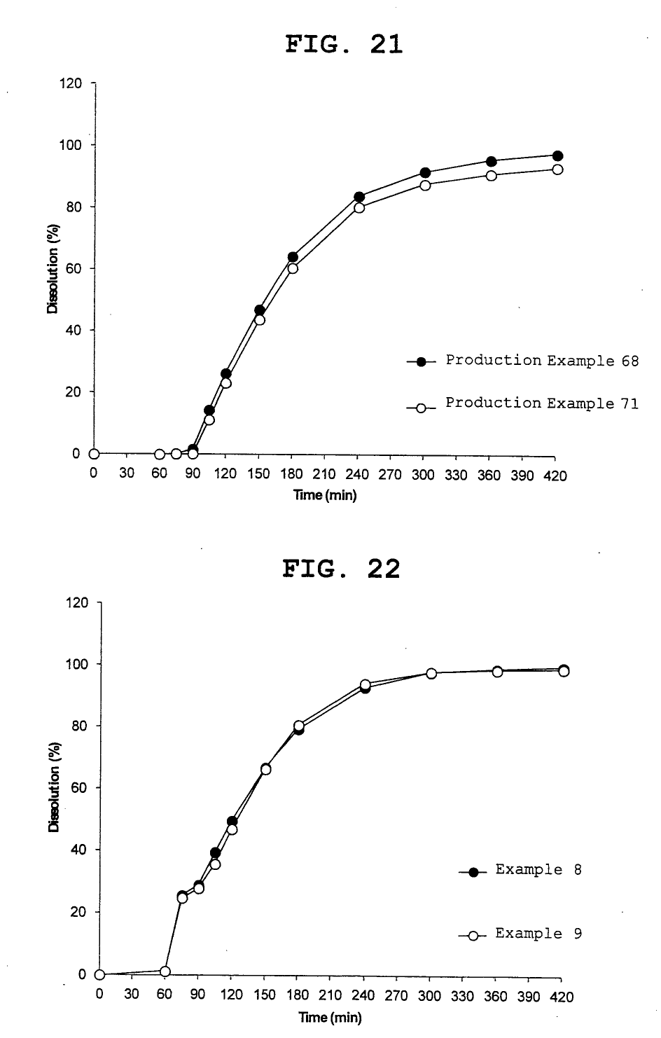

A dissolution test (test method (2)) was performed for the fine granules obtained in Production Examples 68 and 71. The results are shown in FIG. 21.

### Production Example 72

### Production of Outer Layer Component-Granulated Powder

Mannitol (42670 g), low-substituted hydroxypropyl cellulose (L-HPC-33, 7460 g), crystalline cellulose (7460 g) and crospovidone (3730 g) were charged in a fluidized bed granulator (FD-WSG-60, manufactured by POWREX CORPORATION), and they were granulated by spraying an aqueous solution of mannitol (3730 g) and citric acid (746.0 g) in purified water (20400 g) and dried to give a granulated powder.

[Composition of outer layer component-granulated powder
(380.308 mg)]

| | | |
|---|---|---|
| mannitol | 268.196 | mg |
| low-substituted hydroxypropyl cellulose | 43.12 | mg |
| crospovidone | 21.56 | mg |
| crystalline cellulose | 43.12 | mg |
| citric acid | 4.312 | mg |
| total | 380.308 | mg |

### Production Example 73

### Production of Outer Layer Component-Granulated Powder

Mannitol (42650 g), low-substituted hydroxypropyl cellulose (L-HPC-33, 7485 g), crystalline cellulose (7485 g) and crospovidone (3742 g) were charged in a fluidized bed granulator (FD-WSG-60, manufactured by POWREX CORPORATION), and they were granulated by spraying an aqueous solution of mannitol (3742 g) and citric acid (748.5 g) in purified water (20470 g) and dried to give a granulated powder.

[Composition of outer layer component-granulated powder
(404.028 mg)]

| | | |
|---|---|---|
| mannitol | 284.636 | mg |
| low-substituted hydroxypropyl cellulose | 45.92 | mg |
| crospovidone | 22.96 | mg |
| crystalline cellulose | 45.92 | mg |
| citric acid | 4.592 | mg |
| total | 404.028 | mg |

### Example 8

### Production of Orally Disintegrating Tablet

The mannitol-coated fine granules (13060 g) obtained in Production Example 41, the mannitol-coated fine granules (44540 g) obtained in Production Example 68, the outer layer component-granulated powder (59710 g) obtained in Production Example 72, sucralose (1934 g), flavor (STRAWBERRY DURAROME) (677.0 g) and magnesium stearate (967.1 g) were mixed by using a tumbling mixer (TM-400S, SHOWA KAGAKU KIKAI CO., LTD.) to give a mixed powder. The obtained mixed powder (120900 g) was tableted by using a rotary tableting machine (AQUA0836SS2JII (Roman number), Kikusui Seisakusho Ltd.) (770 mg/tablet, a 13 mmφ punch, flat-faced with beveled edge, tableting pressure 28.0 kN) to give the orally disintegrating tablet (770 mg) containing compound X (30 mg) of the present invention.

[Composition of orally disintegrating tablet (770 mg)]

| | | |
|---|---|---|
| mannitol-coated fine granules | 83.2 | mg |
| (Production Example 41) | | |
| mannitol-coated fine granules | 283.7 | mg |
| (Production Example 68) | | |
| outer layer component-granulated powder | 380.308 | mg |
| (Production Example 72) | | |
| sucralose | 12.32 | mg |
| flavor | 4.312 | mg |
| magnesium stearate | 6.16 | mg |
| total | 770 | mg |

The hardness and the disintegration time of the obtained tablet were 42 N and 30 seconds, respectively. The dissolution rate of the obtained tablet in 0.1N HCl in 1 hour was 1.2%, showing superior acid resistance.

### Example 9

### Production of Orally Disintegrating Tablet

The mannitol-coated fine granules (12230 g) obtained in Production Example 41, the mannitol-coated fine granules (45350 g) obtained in Production Example 71, the outer layer component-granulated powder (59390 g) obtained in Production Example 73, sucralose (1929 g), flavor (STRAWBERRY DURAROME) (675.0 g) and magnesium stearate (964.3 g) were mixed by using a tumbling mixer (TM-400S, SHOWA KAGAKU KIKAI CO., LTD.) to give a mixed powder. The obtained mixed powder (120500 g) was tableted by using a rotary tableting machine (AQUA0836SS2JII (Roman number), Kikusui Seisakusho Ltd.) (820 mg/tablet, a 13 mmφ punch, flat-faced with beveled edge, tableting pressure 27.0 kN) to give the orally disintegrating tablet (820 mg) containing compound X (30 mg) of the present invention.

[Composition of orally disintegrating tablet (820 mg)]

| | | |
|---|---|---|
| mannitol-coated fine granules | 83.2 | mg |
| (Production Example 41) | | |
| mannitol-coated fine granules | 308.5 | mg |
| (Production Example 71) | | |
| outer layer component-granulated powder | 404.028 | mg |
| (Production Example 73) | | |
| sucralose | 13.12 | mg |
| flavor | 4.592 | mg |
| magnesium stearate | 6.56 | mg |
| total | 820 | mg |

The hardness and the disintegration time of the obtained tablet were 41 N and 30 seconds, respectively. The dissolution rate of the obtained tablet in 0.1N HCl in 1 hour was 1.2%, showing superior acid resistance.

### Experimental Example 22

A dissolution test (test method (2)) was performed for the formulations obtained in Examples 8 and 9. The results are shown in FIG. 22.

### INDUSTRIAL APPLICABILITY

The orally disintegrating tablet of the present invention comprising fine granules including lansoprazole can suppress the dissolution of lansoprazole in the presence of acid, for example, in the stomach, to achieve a desired dissolution profile of lansoprazole. In addition, since the formulation can control the release of lansoprazole for a long time, a therapeutically effective concentration can be maintained for a prolonged time. Therefore administration frequency can be reduced, and an effective treatment with a small dose can be ensured, and effects such as reduction of side effects caused by the rise of blood concentration and the like can be achieved. Since the formulation shows superior disintegration property or dissolution property in the oral cavity, it is used for the treatment or prophylaxis of various diseases as a formulation conveniently taken by elderly persons and children even without water. In addition, since the fine granules including the pharmaceutically active ingredient having a size preventing dusty texture are blended, a formulation, which is smooth and comfortable in the mouth, can be provided.

This application is based on a U.S. provisional patent application No. 61/427,384, the contents of which are incorporated in full herein.

1. An orally disintegrating tablet comprising
(i) fine granules showing controlled release of a pharmaceutically active ingredient, which comprises fine granules containing a pharmaceutically active ingredient and a coating layer comprising a methacrylic acid/methyl acrylate/methyl methacrylate copolymer, wherein the fine granules containing a pharmaceutically active ingredient are coated with more than 80 wt % and not more than 300 wt % of the copolymer, and
(ii) fine granules showing controlled release of a pharmaceutically active ingredient, which comprises the pharmaceutically active ingredient and a coating layer comprising (a) an ethyl acrylate/methyl methacrylate copolymer, and (b) one or more kinds of polymers selected from the group consisting of methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate, wherein the fine granules (i) and fine granules (ii) have an average particle size of not more than 500 μm, and the pharmaceutically active ingredient is lansoprazole or an optically active form thereof or a salt thereof.

2. An orally disintegrating tablet comprising
(i) fine granules showing controlled release of a pharmaceutically active ingredient, which comprises a pharmaceutically active ingredient and a coating layer comprising (a) a methacrylic acid/methyl acrylate/methyl methacrylate copolymer, and (b) one or more kinds of polymers selected from the group consisting of an ethyl acrylate/methyl methacrylate copolymer, polyvinyl acetate and ethylcellulose, and
(ii) fine granules showing controlled release of a pharmaceutically active ingredient, which comprises a pharmaceutically active ingredient and a coating layer comprising (a) an ethyl acrylate/methyl methacrylate copolymer, and (b) one or more kinds of polymers selected from the group consisting of methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate, wherein the fine granules (i) and fine granules (ii) have an average particle size of not more than 500 μm, and the pharmaceutically active ingredient is lansoprazole or an optically active form thereof or a salt thereof.

3. The orally disintegrating tablet according to claim 1, wherein the coating layers of fine granules (i) and (ii) comprise a plasticizer.

4. The orally disintegrating tablet according to claim 1, wherein the coating layer of fine granules (i) has a coating thickness of 35-70 μm.

5. The orally disintegrating tablet according to claim 1, wherein the pharmaceutically active ingredient is an optically active R form of lansoprazole.

6. The orally disintegrating tablet according to claim 1, further comprising an additive.

7. The orally disintegrating tablet according to claim 6, wherein the additive is a water-soluble sugar alcohol.

8. The orally disintegrating tablet according to claim 1, wherein the coating layers of fine granules (i) and (ii) are formed on an intermediate layer.

9. The orally disintegrating tablet according to claim 1, wherein the coating layer comprising polyethylene glycol, (a) an ethyl acrylate/methyl methacrylate copolymer and (b) one or more kinds of polymers selected from the group consisting of methacrylic acid/ethyl acrylate copolymer, hypromellose phthalate, carboxymethylethylcellulose, polyvinyl acetate phthalate, hydroxypropyl methylcellulose acetate succinate and cellulose acetate phthalate is further formed on each coating layer of fine granules (i) and fine granules (ii).
